(19) United States
(12) Patent Application Publication
(10) Pub. No.: US 2003/0008807 A1
(43) Pub. Date: Jan. 9, 2003

(54) Novel signaling pathway for the production of inflammatory pain and neuropathy

(75) Inventors: Jon David Levine, San Francisco, CA (US); Robert O. Messing, Foster City, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/173,332

(22) Filed: Jun. 14, 2002

Related U.S. Application Data
(60) Provisional application No. 60/298,491, filed on Jun. 14, 2001.

(51) Int. Cl. A61K 31/00; G01N 33/567

(52) U.S. Cl. 514/1; 435/7.21

(57) ABSTRACT

This invention pertains to the discovery of a novel pathway that mediates hyperalgesia, neuropathic pain, and inflammatory pain. This pathway is a third independent pathway that involves activation of extracellular signal-regulated kinases (ERKs) 1 and 2. The pathway comprises a Ras-MEK-ERK1/2 cascade that acts independent of PKA or PKCε as a novel signaling pathway for the production of inflammatory (and neuropathic) pain. This pathway presents numerous targets for a new class of analgesic agents.

### CROSS-REFERENCE TO RELATED APPLICATIONS

This application claims priority to and benefit of U.S. provisional patent application Serial No. 60/298,491, filed Jun. 14, 2001, which is incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported in part by National Institutes of Health Grants NR 04880 and NS 21647. The government of the United States of America may have certain rights in this invention.

### FIELD OF THE INVENTION

This invention pertain to the field of analgesia. In particular, this invention pertains to the discovery of a new pathway that mediates hyperalgesia, inflammatory pain, and neuropathic pain and to methods of screening for agents that inhibit such pain. Related methods and compositions of reducing or lessening pain with inhibitors to this pain pathway are also provided.

### BACKGROUND OF THE INVENTION

Pain is a perception based on signals received from the environment and transmitted and interpreted by the nervous system. Noxious stimuli such as heat and touch cause specialized sensory receptors in the skin to send signals to the central nervous system (“CNS”). This process is called nociception, and the peripheral sensory neurons that mediate it are nociceptors. Depending on the strength of the signal from the nociceptor(s) and the abstraction and elaboration of that signal by the CNS, a person may or may not experience a noxious stimulus as painful.

Thus, pain serves a protective function, when the perception of pain is properly calibrated to the intensity of the stimulus. However, certain types of tissue damage cause a phenomenon, known as hyperalgesia or pronociception, in which relatively innocuous stimuli are perceived as intensely painful because the person's pain thresholds have been lowered. Both inflammation and nerve damage can induce hyperalgesia. Thus, persons afflicted with inflammatory conditions, such as sunburn, osteoarthritis, colitis, carditis, dermatitis, myositis, neuritis, collagen vascular diseases (which include rheumatoid arthritis and lupus) and the like, often experience enhanced sensations of pain. Similarly, trauma, surgery, amputation, abscess, causalgia, collagen vascular diseases, demyelinating diseases, trigeminal neuralgia, cancer, chronic alcoholism, stroke, thalamic pain syndrome, diabetes, herpes infections, acquired immune deficiency syndrome (“AIDS”), toxins and chemotherapy cause nerve injuries that result in excessive pain. The reduced pain thresholds, characteristic of hyperalgesia, are due to alterations in the way that nociceptors adjacent to the inflammation or damaged nerves respond to noxious stimuli.

If the mechanisms by which nociceptors transduce external signals under normal and hyperalgesic conditions were better understood, it might be possible to identify processes unique to hyperalgesia that, when interrupted, could inhibit the lowering of the pain threshold and thereby lessen the amount of pain experienced. Since such a treatment for chronic pain would act at the level of the sensory afferent neurons, it would bypass the problems associated with drugs that act on the CNS. If the treatment incapacitated a transduction pathway specific to nociceptors and/or not involved in mediating other signals, then the potential for treatment-induced side effects would be small.

For example, inflammatory pain, characterized by a decrease in mechanical nociceptive threshold (hyperalgesia) arises through actions of inflammatory mediators, many of which sensitize primary afferent nociceptors via G-protein coupled receptors. Two signaling pathways, one involving protein kinase a (PKA) and one involving the epsilon isozyme of protein kinase C (PKCε0), have been implicated in primary afferent nociceptor sensitization. However, these pathways do not fully account for inflammatory mediator-induced hyperalgesia.

The present invention relates to a third pain pathway involving activation of extracellular signal-regulated kinases (ERKs) 1 and 2 through, e.g., inflammatory mediator-induced hyperalgesia. Methods and compositions related to inhibitors of this third pain pathway are provided. A fuller understanding of the invention will be provided by review of the following.

### SUMMARY OF THE INVENTION

This invention pertains to the discovery of a novel pathway that mediates hyperalgesia, neuropathic pain, and inflammatory pain. This pathway is a third independent pathway that involves activation of extracellular signal-regulated kinases (ERKs) 1 and 2. Epinephrine, which induces hyperalgesia by direct action at β₂-adrenergic receptors and on primary afferent nociceptors stimulated phosphorylation of ERK1/2 in cultured rat DRG cells. This was inhibited by a β₂-adrenergic receptor blocker and by an inhibitor of mitogen and extracellular signal-regulated kinase kinase (MEK) which phosphorylates and activates ERK1/2. Inhibitors of G_{i/o}-proteins, Ras farnesyltransferases, and MEK decreased epinephrine-induced hyperalgesia.

In similar fashion, phosphorylation of ERK1/2 was also decreased by these inhibitors. Local injection of dominant active MEK produced hyperalgesia that was unaffected by PKA or PKCε inhibitors. Conversely, hyperalgesia produced by agents that activate PKA or PKCε was unaffected by MEK inhibitors. We conclude that in primary afferent nociceptors, a Ras-MEK-ERK1/2 cascade acts independent of PKA cascade or PKCε cascade as a novel signaling pathway for the production of inflammatory (and neuropathic) pain. This pathway presents numerous targets for a new class of analgesic agents.

Thus, in one embodiment, this invention provides methods of screening for an inhibitor of inflammatory or neuropathic pain, said method comprising: assaying a test agent for the ability to inhibit pain that is mediated by a Ras-mitogen-activated protein kinase/extracelluar-signal related kinase kinase (MEK)-ERK1/2 cascade. In certain embodiments, the assaying comprises: providing a neurological tissue preparation; contacting said neurological tissue with an agent that induces hyperalgesia, e.g., epinephrine or NGF; contacting said neurological tissue with the test agent; and assaying, e.g., ERK expression or activity wherein a decrease in the ERK expression or activity as compared to a control indicates that said test agent inhibits inflammatory or neuropathic pain, assaying MEK kinase expression or activity wherein a decrease in the MEK kinase expression or activity as compared to a control indicates that said test agent inhibits inflammatory or neuropathic pain, assaying Ras protein expression or activity wherein a decrease in the Ras protein expression or activity as compared to a control indicates that said test agent inhibits inflammatory or neuropathic pain, assaying Gi/o protein expression or activity wherein a decrease in the Gi/o protein expression or activity as compared to a control indicates that said test agent inhibits inflammatory or neuropathic pain, assaying nociceptive threshold activity wherein a decrease in a percentage decrease in the nociceptive threshold activity as compared to a control indicates that said test agent inhibits inflammatory or neuropathic pain or assaying for combinations of the above. In certain embodiments, the control comprises absence of said test agent or said test agent present at a lower concentration. In one embodiment, the neurological preparation is a dorsal root ganglion preparation.

In another embodiment, assaying comprises: selecting as the test agent, a compound that modulates the activity of the Ras-MEK-ERK 1/2 cascade; and, administering the test agent to a subject to determine whether pain is modulated, wherein the test agent modulates pain in the subject by modulating the expression or activity of at least one member of the Ras-MEK-ERK 1/2 cascade.

In still another embodiment, assaying comprises assaying for protein expression of a member of the Ras-MEK-ERK 1/2 cascade via a method selected from the group consisting of: a capillary electrophoresis, a Western blot, mass spectroscopy, ELISA, immunochromatography, and immunohistochemistry. In another embodiment, assaying comprises assaying for activity via a method selected from the group consisting of: a phosphorylation assay, an immunoassay, a binding assay, a withdrawal threshold assay and a nociceptive threshold assay, e.g., a withdrawal threshold assay.

In one embodiment, methods for screening further comprise assaying said test agent for inhibitory or agonistic activity at PKA cascade or PKCε cascade where a lack of activity of said test agent at the PKA cascade or PKCε cascade indicates that said test agent is pathway-specific.

In certain embodiments, the test agent is contacted directly to the member of the Ras-MEK-ERK 1/2 cascade. The test agent can also be contacted to a cell containing the Ras-MEK-ERK 1/2 cascade. In another embodiment, the test agent is contacted to an animal comprising a cell containing the Ras-MEK-ERK 1/2 cascade.

The invention also provides for methods for screening for inhibitors of all three pathways, where the method comprises assaying a test agent for the ability to modulate activity of a tetrodotoxin-resistant sodium current wherein inhibition of the tetrodotoxin-resistant sodium current indicates that said test agent inhibits inflammatory or neuropathic pain mediated by PKA cascade, PKCε cascade and RAS-MEK-ERK 1/2 cascade. In certain embodiments, the assaying comprises: contacting a neurological tissue preparation with an agent that induces hyperalgesia (e.g., epinephrine, NGF, bradykinin, norepinephrine, prostaglandin E₂ and the like); contacting said neurological tissue preparation with the test agent; and assaying for modulation of the activity of the tetrodotoxin-resistant sodium current. In certain embodiments, the neurological tissue preparation comprises a neuronal culture. In another embodiment, the neuronal culture is a primary neuronal culture. In still another embodiment, the neurological preparation is a dorsal root ganglion preparation.

This invention also provides a method for prescreening for an agent that inhibits Ras-MEK-ERK 1/2 cascade, said method comprising: contacting a member of the Ras-MEK-ERK 1/2 cascade with a test agent; and, detecting specific binding of said test agent to said member of the Ras-MEK-ERK 1/2 cascade, wherein specific binding indicates that said agent is a candidate inhibitor of the Ras-MEK-ERK 1/2 cascade. In certain embodiments, the detecting is via a method selected from the group consisting of capillary electrophoresis, a Western blot, mass spectroscopy, ELISA, immunochromatography, and immunohistochemistry. In one embodiment, the test agent can be contacted directly to the member of the Ras-MEK-ERK 1/2 cascade. In another embodiment, the test agent is contacted to a cell containing the Ras-MEK-ERK 1/2 cascade.

The invention also provides methods for desensitizing nocioceptors by inhibiting the Ras-MEK-ERK1/2 cascade. In certain embodiments, the inhibiting comprises inhibiting, e.g., MEK kinase expression or activity, ERK expression or activation, Ras expression or activity, Gi/o expression or activity, β-adrenergic receptor mediated expression or activation of ERK, NGF-mediated expression or activation of ERK, bradykinin-mediated expression or activation of ERK and the like.

The invention also provides methods for reducing or lessening pain by administering an inhibitor of the Ras-MEK-ERK 1/2 cascade. For example, a method comprises: administering to a subject in need thereof, an effective amount of an inhibitor of a Ras-MEK-ERK 1/2 cascade, wherein the inhibitor causes the reducing or lessening of pain by interfering with the Ras-MEK-ERK 1/2 cascade. In certain embodiments, the administration results in the subject having decreased hyperalgesia. In another embodiment, the inhibitor is administered locally or is administered systemically. In certain embodiments, the inhibitor is in a pharmaceutical formulation.

In one embodiment, the subject has inflammatory pain (e.g., acute or chronic). The inflammatory pain can be due to a condition from the group consisting of : sunburn, osteoarthritis, colitis, carditis, dermatis, myositis, neuritis and collagen vascular disease. In another embodiment, the subject has neuropathic pain. The neuropathic pain can involve a peripheral nerve or a central nerve. The neuropathic pain can be due to a neuropathy, e.g., radiculopathy, mononeuropathy, mononeuropathy multiplex, polyneuropathy, plexopathy and the like. The neuropathic pain can also be due to, e.g., a condition selected from the group consisting of: causalgia, diabetes, collagen vascular disease, trigeminal neuralgia, spinal cord injury, brain stem injury, thalamic pain syndrome, cancer, chronic alcoholism, stroke, cancer, abscess, demyelinating disease, herpes infection, and AIDS, or can be due to one or more of the following selected from the group consisting of: trauma, surgery, amputation, toxin, and chemotherapy.

Typically, the subject is human. In another embodiment, the subject is a non-human mammal (e.g., a primate, a mouse, a pig, a cow, a cat, a goat, a rabbit, a rat, a guinea pig, a hamster, a horse, a sheep, a dog, a cat and the like). The subject can be male or female.

In certain embodiments, the inhibitor is an inhibitor of a β2 adrenergic receptor, e.g., an inverse agonist, such as ICI 118,551 or an antagonist, such as propanolol. In another embodiment, the inhibitor is an inhibitor of Gi/o protein activity, e.g., an isoprenylation inhibitor, pertussis toxin, perillic acid and the like. In still yet another embodiment, the inhibitor is an inhibitor of Ras activity, e.g., famesyltransferase, FTase I and the like. In another embodiment, the inhibitor is an inhibitor of MEK activity, e.g., U0126, PD98059 and the like. The inhibitor can also be an inhibitor of ERK 1/2 activity.

In still another embodiment, the inhibitor inhibits catalytic activity of a member of the Ras-MEK-ERK 1/2 cascade or the inhibitor inhibits intracellular translocation of a member of the Ras-MEK-ERK 1/2 cascade. The inhibitor can act directly on a member of the Ras-MEK-ERK 1/2 cascade or can act indirectly on a member of the Ras-MEK-ERK 1/2 cascade. In certain embodiments, the inhibitor is membrane-permeable.

In one embodiment, the methods of reducing or lessening pain further comprise administering at least one compound from the group consisting of: an inhibitor of cAMP, a nonsteroidal anti-inflammatory drug, a local anesthetic, an anticonvulsant, an antidepressant, and an opiod. In another embodiment, the method of reducing or lessening pain further comprise administering an inhibitor of a prostaglandin E₂ cascade to said mammal in a concentration sufficient to inhibit prostaglandin E₂ hyperalgesia. For example, an inhibitor of the prostaglandin E₂ cascade is a nitric oxide synthetase (NOS) inhibitor, such as NG-methyl-L-arginine (L-MNA). In still another embodiment, the methods further comprise administering an inhibitor of a protein kinase A (PKA) cascade and/or protein kinase Cε (PKCε) cascade to said subject in a concentration sufficient to inhibit the PKA cascade and/or the PKCε cascade. For example, the inhibitor of the PKA cascade can be, e.g., a Walsh inhibitor peptide (WIPTIDE), a H89 and the like. In certain embodiments, the inhibitor is a PKCε inhibitor, e.g., protein kinase C epsilon inhibitor peptide (PKCε-I), a calphostin C, an εV1-2 and the like.

This invention also pertains to the identification of gender differences in the signaling of inflammatory pain. This second messenger signaling pathway identified herein and present in peripheral nociceptors contributes inflammatory pain along with protein kinase A (PKA) and protein kinase C epsilon (PKCε) in males, but contributes to a much greater extent in females. Drugs that inhibit this pathway represent a novel class of analgesics. This class of analgesics is particularly well suited for, e.g., inflammatory pain, which is much more prevalent in women (e.g. rheumatoid arthritis, systemic lupus etc).

In addition, in one embodiment, the invention provides a method of decreasing hyperalgesia or pain, e.g., inflammatory pain, neuropathic pain, pain of a type produced by formalin, and the like, in a mammal, said method comprising administering estrogen or an estrogen analog or agonist to said mammal in a concentration sufficient to inhibit contributions of β adrenegic receptor, e.g., β2 adrenegic receptor, mediated PKA and/or PKCε to pain signaling. In certain embodiments, the estrogen analog or agonist is selected from the group consisting of: an estradiol, an estrone, an ethinyl estradiol, a diethylstilbestrol, a mestranol, an estrone, a conjugated estrogen, a chlorotrianisene and analogs thereof. The mammal can be a female mammal or a male mammal.

In certain embodiments, the methods of decreasing hyperalgesia or pain further comprising administering an inhibitor of a Ras-MEK-ERK 1/2 cascade to said mammal in a concentration sufficient to inhibit the Ras-MEK-ERK 1/2 cascade. In one embodiment, the inhibitor of the Ras-MEK-ERK 1/2 cascade is a MEK inhibitor, e.g., PD 98059, U0126 and the like. In another embodiment, the Ras-MEK-EKR 1/2 inhibitor is a β2-adrenergic receptor inhibitor, e.g., an inverse agonist (e.g., ICI 118,551) or antagonist (e.g., propanolol).

In still another embodiment, the methods further comprise administering an inhibitor of a prostaglandin E₂ cascade to said mammal in a concentration sufficient to inhibit prostaglandin E₂ hyperalgesia. In certain embodiments, the inhibitor is a NOS inhibitor, e.g., NG-methyl-L-arginine (L-MNA).

The invention also provides for compositions. These include a composition comprising an inhibitor of the Ras-MEK-ERK 1/2 cascade. In certain embodiments, the composition includes an agent that inhibits activity or expression of a component of a Ras-MEK-ERK 1/2 cascade said component being selected from the group consisting of of ERK, MEK kinase, Ras protein, and a Gi/o protein. Optionally, an analgesic agent is also included (e.g., an opioid, a local anesthetic, an anticonvulsant and an antidepressant, a nonsteroidal anti-inflammatory drug (NSAID), such as aspirin, ibuprofen, and indomethacin and the like) the analgesic agent having a mechanism of action other than inhibition of the Ras-MEK-ERK 1/2 cascade. In certain embodiments, the composition further comprises an inhibitor of PKA cascade, an inhibitor of the PKCε cascade or both the inhibitor of the PKA cascade and the inhibitor of the PKCε cascade. In certain embodiments, the composition comprises a unit doage formulation.

In certain embodiments, the composition is present in a pharmacologically acceptable excipient. In one embodiment, the composition with the pharmacologically acceptable excipient is formulated for transdermal administration, and/or optionally, it is formulated for topical administration. The composition with the pharmacologically acceptable excipient can also be formulated as a cream, lotion or emulsion.

Kits for reducing pain are also provided. For example, such kits comprise a container containing an inhibitor of a Ras-MEK-ERK 1/2 cascade. In one embodiment, the kit further comprises instructional materials teaching the use of an inhibitor of a Ras-MEK-ERK 1/2 cascade to reduce pain.

Definitions

The terms “active agent,” “drug” and “pharmacologically active agent” are used interchangeably herein to refer to a chemical material or compound that induces a desired effect.

The terms “acute pain” and “chronic pain” refer to types of pain. Acute pain is experienced soon (e.g., within about 48 hours, within about 24 hours or within about 12 hours) after the occurrence of the event (such as inflammation or nerve injury) that led to such pain. There is a significant time lag between the experience of chronic pain and the occurrence of the event that led to such pain. Such time lag is, e.g., at least about 48 hours after such an event, at least about 96 hours after such event, or at least about one week after such event.

An term “analgesic” refers to a molecule or a combination of molecules that causes a lessening or reduction in pain.

The term “antagonist” refers to an agent, e.g., a drug or a compound, that opposes the physiological effects of another. It can act directly or indirectly on expression or activity. At the receptor level, it is a chemical entity that opposes the receptor-associated responses normally induced by another bioactive agent. A drug is any substance presented for treating, curing, or preventing disease in a humans being or animals. A drug may also be used for making a medical diagnosis or for restoring, correcting, or modifying physiological function.

The term “effective amount” refers to an amount that results in the lessening of pain. Such effective amount will vary from subject to subject depending on the subject's normal sensitivity to pain, its height, weight, age, and health, the source of the pain, the mode of administering the inhibitor of Ras-MEK-ERK 1/2 cascade, the particular inhibitor administered, and other factors. As a result, it is advisable to empirically determine an effective amount for a particular subject under a particular set of circumstances.

The term “expression” refers to protein expression, e.g., mRNA and/or translation into protein. The term “activity” refers to the activity of a protein. Activities include but are not limited to phosphorylation, signaling activity, activation, catalytic activity, protein-protein interaction, transportation, etc. The expression and/or activity can increase, or decrease. Expression and/or activity can be activated or inhibited directly or indirectly.

The term “hyperalgesia” refers to the excessive sensitiveness or sensibility to pain. Hyperalgesia can occur both at the site of tissue damage and in the surrounding undamaged areas. One type of characterization of hyperalgesia is a decrease in mechanical nociceptive threshold.

The term “inflammatory pain” refers to pain that results from inflammation, wherein inflammation is the reaction of living tissues to injury, infection or irritation. Anything that stimulates the inflammatory response is said to be inflammatory.

The term “modulator” refers to an agent that alters (e.g. upregulates or downregulates) expression or activity of a pathway and/or one or more components of a pathway.

The term “inhibit” when used in reference to activity (e.g. of an enzyme) refers to a partial or complete reduction in activity of the subject agent (e.g. enzyme).

The term “inhibitor” refers to a molecule or group of molecules that interfers with: (1) the expression, modification, regulation or activation of a member of the Ras-MEK-ERK 1/2 cascade, (2) one or more normal functions of a member of the Ras-MEK-ERK 1/2 cascade, or (3) the expression, modification, regulation or activation of a molecule acting downstream of the Ras-MEK-ERK 1/2 cascade.

The term “inverse agonist” (also called negative antagonist) refers an agent, e.g., a drug or a compound, which acts at the same receptor as that of an agonist, yet produces an opposite effect.

The term “neuropathic pain” refers to pain that results from a disturbance of function or pathologic change in a nerve, in one nerve mononeuropathy, in several nerves, mononeuropathy multiplex, if diffuse and bilateral, polyneuropathy.

The term “PKA cascade” refers to a pain signaling pathway that involves an agent, e.g., epinephrine or an analog thereof, a β-adrenegeric receptor, e.g., β2-adrenergic receptor, or another cell surface receptor, and protein kinase A. This can lead to modulation of the activity of a tetrodotoxin-resistant sodium current.

The term “PKCε cascade” refers to a pain signaling pathway that involves an agent, e.g., epinephrine, bradykinin, nerve growth factor (NGF), epidermal growth factor (EGF) or an analog thereof, a β-adrenegeric receptor, e.g., β2-adrenergic receptor, or another cell surface receptor, e.g., NGF receptor, and protein kinase Ce. This can lead to modulation of the activity of a tetrodotoxin-resistant sodium current.

The term “prostanglandin E₂ cascade” refers to a pain signaling pathway that involves an agent, e.g., prostanglandin E₂ (PGE₂), serotonin, adenosine or an analog thereof, and activation of PKA which is facilitated by nitric oxide (NO). This can lead to modulation of the activity of a tetrodotoxin-resistant sodium current.

The terms “Ras-MEK-ERK 1/2 cascade” or “Ras-MEK-ERK 1/2 pathway” refers to a pain signaling pathway can be activated by the action of epinephrine on a a β-adrenergic receptor, e.g., β2-adrenergic receptor. Signaling of the pathway is mediated by Gi/o—protein(s0, a Ras protein, a mitogen-activated protein kinase/extracellular-signal related kinase kinase (MEK), and activation of extracellular signal-regulated kinases (ERKs) 1 and/or 2. This can lead to modulation of the activity of a tetrodotoxin-resistant sodium current. This pathway is typically independent of the PKA cascade and PKCε cascade.

The term “reducing or lessening pain” refers to a process by which the level of pain a subject perceives is reduced relative to the level of pain the subject would have perceived were it not for the intervention. Where the subject is a person, the level of pain the person perceives can be assessed by asking him or her to describe the pain or compare it to other painful experiences. Alternatively, pain levels can be calibrated by measuring the subject's physical responses to the pain, such as the release of stress-related factors or the activity of pain-transducing nerves in the peripheral nervous system or the CNS. One can also calibrate pain levels by measuring the amount of a well characterized analgesic required for a person to report that no pain is present or for a subject to stop exhibiting symptoms of pain. Lessening pain can result from increasing the threshold at which a subject experiences a given stimulus as painful. It can result from inhibiting hyperalgesia, the heightened sensitivity to a noxious stimulus, and such inhibition can occur without impairing nociception, the subject's normal sensitivity to a noxious stimulus.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

### BRIEF DESCRIPTION OF THE DRAWINGS

FIGS. 1A-1D show that epinephrine stimulates ERK1/2 phosphorylation in DRG neurons. FIG. 1A: ERK1/2 immunoreactivity present in cell bodies of neurons in freshly isolated DRG. The top shows incubation with anti-ERK1/2, whereas the bottom shows loss of immunoreactivity after preincubation of antibody with excess of peptide antigen. Scale bar, 100 μm. FIG. 1B: DRG cultures were treated with epinephrine for the indicated times and then processed for analysis of phospho-ERK1/2 immunoreactivity by Western analysis. Some cells were treated instead with 50 ng/ml NGF for 5 min as a positive control. Blots were then stripped and probed with anti phospho-ERK1/2 antibody. Representative Western blot demonstrating NGF and epinephrine stimulation of ERK1/2 phosphorylation. FIG. 1C: Mean±SE values (n=7-23) for phospho-ERK1/2 immunoreactivity normalized to total ERK1/2 immunoreactivity. *p<0.05 by ANOVA and Dunnett's multiple comparison test. FIG. 1D: Representative Western blot showing concentration dependence of epinephrine-induced ERK1/2 phosphorylation.

FIGS. 2A-2C show that epinephrine-induced phosphorylation of ERK1 (gray bars) and ERK2 (black bars) is reduced by inhibitors of β₂-adrenergic receptors and MEK and is independent of PKA and PKCε. FIG. 2A: DRG cultures were treated with 1 μM epinephrine (Epi; n=9) for 5 min in the absence or presence of (A) ICI 118,551 (ICI; 100 nM; n=7) or U0126 (U; 10 μM; n=2). FIG. 2B: Cultures were treated with 1 μM epinephrine (Epi; n=11) for 5 min in the absence or presence of H89 (1 μM; n=5) or calphostin C (Cal; 1 μM; n=7). FIG. 2C: DRGs cultured from PKCε wild-type (WT) or knock-out (KO) mice were treated with 1 μM epinephrine for 5 min (n=3). Data are mean ±SE values. *p<0.05 compared with phospho-ERK1 in epinephrine-treated cells; **p<0.05 compared with phospho-ERK2 measured in epinephrine-treated cells (one-way ANOVA and Tukey's multiple comparison test).

FIGS. 3A-3D show that epinephrine-induced hyperalgesia is mediated by MEK, independent of PKA and PKCε. The mean ±SE baseline threshold before drug administration was 108.0±0.5 gm (n=162). FIG. 3A: Rats were treated intradermally with epinephrine (Epi; 100 ng; n=12), U0126 (U; 1 μg; n=6), U0126 plus epinephrine (U/Epi; n=12), PD98059 (PD; 1 μg; n=6), and PD98059 plus epinephrine (PD/Epi; n=12). FIG. 3B, Rats were treated intradermally with PGE2 (100 ng; n=6), U0126 plus PGE₂ (U/PGE2; n=6), and PD98059 plus PGE2 (PD/PGE₂; n=6). FIG. 3C: Rats were treated intradermally with the PKCε agonist ψεRACK (ψεR; 1 μg; n=6), U0126 plus PKCε agonist (U/ψεR; n=6), and PD98059 plus PKCε agonist (PD/ψεR; n=6). FIG. 3D: Rats were treated intradermally with active MEK (MEK+;0.5 U; n=12), inactive MEK (MEK−; 1 μg; n=6), PKCε inhibitor (εV1-2; 1 μg; n=6), PKCε inhibitor plus active MEK (εV1-2/MEK+; n=6), WIPTIDE (WIP; 1 μg; n=6), and WIPTIDE plus active MEK (WIP/MEK+; n=6). *p<0.05 by one-way ANOVA and Newman-Keuls test.

FIGS. 4A-4C show Epinephrine-induced mechanical hyperalgesia is mediated by a Gi/o-protein and Ras. The mean ±SE baseline threshold before drug administration was 108.0±0.5 gm (n=162). FIG. 4A: Rats were treated intradermally with epinephrine (Epi; 100 ng; n=12), pertussis toxin (PTX; 1 μg; n=6), or pertussis toxin plus epinephrine (PTX/Epi; n=8). FIG. 4B: Rats were treated intradermally with epinephrine (Epi; 100 ng; n=12), perillic acid (PER; 1 μg; n=6), or perillic acid plus epinephrine (PER/Epi; n=8). FIG. 4C: Rats were treated intradermally with epinephrine (Epi; 100 ng; n=12), farnesyltransferase inhibitor I (FT; 1 μg; n=6), or farnesyltransferase inhibitor I plus epinephrine (FT/Epi; n=8). *p<0.05 by one-way ANOVA and Newman-Keuls test.

FIG. 5 shows that pertussis toxin (PTX) and farnesyltransferase inhibitor I (FT) inhibit epinephrine-induced ERK1/2 phosphorylation in DRG cultures. DRG cultures were treated with 100 μM pertussis toxin or 1 μM FTase I for 16 hr and then with or without 1 μM epinephrine (Epi) as indicated. Data are mean ±SE values from five to eight experiments. *p<0.05 compared with phospho-ERK1 in epinephrine-treated cells; **p<0.05 compared with phospho-ERK2 measured in epinephrine-treated cells (one-way ANOVA and Newman-Keuls test).

FIGS. 6A and 6B show the effects of epinephrine on paw withdrawal thresholds. FIG. 6A: Dose-dependent effects of intraderrnally injected epinephrine on mechanical paw-withdrawal thresholds in gonad-intact male (filled squares; n=12 paws) and female (filled triangles; n=12 paws) rats. Responses are shown as percentage change from baseline after epinephrine administration. Each point represents mean 6SEM. *P<0.05 (repeated-measures ANOVA followed by Fisher's PLSD post hoc test). FIG. 6B: Gonad-intact male and female rats were injected with 100 ng epinephrine; without (filled bars; n=18 paws) or with (hatched bars; n=8 paws) 1 μg propranolol. *P<0.0001 (unpaired Student's t-test).

FIGS. 7A and 7B show the percentage change from baseline in paw-withdrawal thresholds in (FIG. 7A) male gonad-intact (filled bars) and gonadectomized (hatched bars) and (FIG. 7B) female gonad-intact (filled bars), gonadectomized (hatched bars) and gonadectomized with oestrogen-implanted (cross-hatched bars) rats (n=18 paws in each group). Rats were examined after intradermal injection of 100 ng epinephrine (EPI) alone, or epinephrine plus 1 μg of the PKA inhibitor WIPTIDE (EPI/WIPTIDE; n=6 paws in all groups), epinephrine plus 1 μg of PKCε inhibitor peptide (EPI/PKCε-I; n=6 paws in all groups), and epinephrine plus 1 μg of either of the two MEK inhibitors, PD 98059 (EPI/PD 98059; n=6 or 12 paws in males or females, respectively) and U 0126 (EPI/U 0126; n=6 or 12 paws in males or females, respectively). *P<0.0001, ANOVA and Fisher's PLSD post hoc test.

FIG. 8 shows a schematic diagram of the proposed cellular mechanisms for epinephrine and prostaglandin E₂ hyperalgesia and their modification by oestrogen, studied in gonad-intact and gonadectomized rats. EP-R, EP-type prostaglandin receptor; E(−), E(+), oestrogen loss through gonadectomy or oestrogen replacement, respectively; TTX-R I_{Na+}, tetrodotoxin-resistant sodium current; MEK, mitogen-activated protein kinase/extracellular-signal related kinase kinase second messenger signalling mechanism for β2-adrenergic receptor (β2-AR)-mediated epinephrine-induced hyperalgesia in gonad-intact females.

FIGS. 9A-9D show the effect of WIPTIDE and L-NMA on prostaglandin E₂. (FIG. 9A) Effect of 1 μg WIPTIDE and (FIG. 9C) 1 μg L-NMA on prostaglandin E₂ (PGE₂; 100 ng)-induced decreases in nociceptive threshold. (FIG. 9B and FIG. 9D) Effect of 1 μg L-NMA on epinephrine (EPI; 100 ng)-induced decreases in nociceptive threshold. Studies were performed in (FIGS. 9A, 9B and 9C) gonad-intact and (FIG. 9D) gonadectomized male (filled bars; n=12 paws) and female (hatched bars; n=12 paws) rats. WIPTIDE or L-NMA was coinjected intradermally with PGE₂ or EPI. *P<0.0001 (ANOVA and Fisher's PLSD post hoc test). N.S., not significant.

FIG. 10 shows the effect of 100 ng epinephrine on wild type (filled bars) and PKCε-null (hatched bars) male (M) and female (F) mice. Responses to von Frey hairs at 3.82 N/mm² (36.3 mN) and 4.54 N/mm² (60.3 mN) are shown as mean paw withdrawal frequencies ±SEM (n=8 in all groups). Baseline responses were not different between the groups. *P<0.05 (ANOVA and Fisher's PLSD post hoc test).

FIG. 11 shows the absolute change in paw-withdrawal thresholds in male rats (filled dark bars) and female rats (unfilled bars) with Taxol-induced hyperalgesia. Rats were examined after administration of Taxol alone, or Taxol plus a MEK inhibitor, PD98059 (“MEKI”), along with a control in males and females. The control pain threshold in normal male and female rats was approximately 110 grams, using the Randall-Selitto paw-withdrawal test. Following the administration of Taxol, the threshold fell to approximately 70 grams in both the male and female rats. Administration of the MEK inhibitor PD98059 reversed the Taxol-induced hyperalgesia in male and female rats.

FIG. 12 shows the absolute change in paw-withdrawal thresholds in male rats (filled dark bars) and female rats (unfilled bars) with vincristine-induced hyperalgesia. Rats were examined after administration of vincristine alone, or vincristine plus the MEK inhibitor, PD98059 (“MEKI”), along with a control in males and females. The control pain threshold in normal male and female rats was approximately 110 grams, using the Randall-Selitto paw-withdrawal test. Following the administration of vincristine, the threshold fell to approximately 65 grams in both the male and female rats. Administration of the MEK inhibitor PD98059 reversed the vincristine-induced hyperalgesia in male and female rats.

FIGS. 13A and B show the percentage change in nociceptive threshold verses the number of weeks of alcohol consumption by female rats (FIG. 13A) and male rats (FIG. 13B) with alcohol-induced hyperalgesia. FIG. 13A shows female alcohol-treated rats (filled dark bars) and control treated rats (unfilled bars) following a chronic consumption of a diet in which alcohol replaced calories but not other nutrients, where the threshold fell to approximately 70 grams. Rats were examined after administration of alcohol and the MEK inhibitor, PD98059 (“MEKI”) or U0126. Administration of the MEK inhibitor PD98059 or U0126 almost completely reversed the alcohol-induced hyperalgesia in female rats. FIG. 13B shows male alcohol-treated rats (filled dark bars) and control treated rats (unfilled bars) following a chronic consumption of a diet in which alcohol replaced calories but not other nutrients, where the threshold fell to approximately 70 grams. Rats were examined after administration of alcohol and the MEK inhibitor, PD98059 (“MEKI”) or U0126. Administration of the MEK inhibitor PD98059 or U0126 almost completely reversed the alcohol-induced hyperalgesia in male rats.

### DETAILED DESCRIPTION

This invention pertains to the discovery of a new pathway that mediates neuropathic and inflammatory pain and to methods and compositions for modulating the activity of this pathway. The pathway, designated herein as the Ras-MEK-ERK 1/2 cascade mediates activity (e.g. hyperalgesia induced by direct action of epinephrine at β2-adrenegic receptors) through the activation of ERK. A heterotrimeric Gi- and/or Go-protein, Ras, and MEK also contribute to epinephrine-induced hyperalgesia, independent of PKC or PKA.

Sensory afferent neurons (also known as nociceptors or primary afferent neurons) are a subset of small- and medium-diameter dorsal root ganglion neurons (“DRG neurons”) that extend from the dermis, where their peripheral terminals are located, to the superficial laminae of the dorsal horn, where they synapse with CNS neurons. In sensory afferent neurons, the Ras-MEK-ERK 1/2 cascade is a secondary messenger cascade transducing a response initiated by a noxious stimulus or a hyperalgesia-inducing agent.

This pathway was identified by using inhibitors to other pain pathways, e.g., PKA and PKCε, which did not block the effect of this new pathway. We found that epinepherine, which induces hyperalgesia by direct action at β2-adrenegic receptors on primary afferent nociceptors, activates ERKs in cultured DRG neurons and that a heterotrimeric Gi- and/or Go-protein, Ras, and MEK contribute to epinephrine-induced hyperalgesia, independent of PKCε or PKA.

The Ras-MEK-ERK 1/2 cascade makes a good target to screen for agents that inhibit pain (e.g., inflammatory pain, neuropathic pain, etc). Such agents are expected to be useful therapeutics and/or lead compounds for the development of useful therapeutics in a wide variety of contexts. Methods of screening for such agents are provided. In certain embodiments, the methods comprise: assaying a test agent for the ability to inhibit pain that is mediated by a Ras-mitogen-activated protein kinase/extracelluar-signal related kinase kinase (MEK)-ERK1/2 cascade. In another embodiment, methods for screening for inhibitors of all three pathways (Ras-MEK-ERK 1/2 pathway, PKA pathway, and PKC pathway) are provided.

Moieties that inhibit the Ras-MEK-ERK 1/2 cascade can be utilized to, reduce or eliminate pain in a variety of contexts. Such conditions include, but are not lmited to causalgia, diabetes, collagen vascular disease, trigeminal neuralgia, spinal cord injury, brain stem injury, thalamic pain syndrome, cancer, chronic alcoholism, stroke, cancer, abscess, demyelinating disease, herpes infection, AIDS, trauma, surgery, amputation, toxin, and chemotherapy.

The Ras-MEK-ERK 1/2 cascade identified herein and present in peripheral nociceptors contributes inflammatory pain along with protein kinase A (PKA) and protein kinase C epsilon (PKCε) in males, but contributes to a much greater extent in females. Drugs that inhibit this pathway represent a novel class of analgesics. In addition, in certain embodiments, the invention provides a method of decreasing hyperalgesia or pain, e.g., inflammatory pain, neuropathic pain, pain of a type produced by formalin, and the like, in a mammal, said method comprising administering estrogen or an estrogen analog or agonist to said mammal in a concentration sufficient to inhibit contributions of β2 adrenegic receptor mediated PKA cascade or PKCε cascade to pain signaling.

In addition to various assays and methods provided herein, also provided are novel compositions that modulate (e.g. inhibit) the Ras-MEK-ERK 1/2 cascade.

I. Assays for Screening for Inhibitors of the Ras-MEK-ERK 1/2 Cascade

As indicated above, in one aspect, this invention pertains to the discovery of a new path pathway. In particular this invention pertains to the discovery that the Ras-MEK-ERK 1/2 pathway mediates pain (e.g. neuropathic pain, inflammatory pain, etc.). Thus, the Ras-MEK-ERK 1/2 cascade provides a target to screen for modulators (e.g. upregulators or inhibitorsinhibitors) that can be useful in a wide variety of contexts (e.g. in the treatment of pain or one or more symptoms associated with acute or chronic pain).

The methods typically involve contacting a cell (preferably a cell from or in a neurological tissue) with a test agent and detecting change in expression or activity of one or more components of the Ras-MEK-ERK 1/2 cascade (pathway). Such components incude, but are not limited to ERK, MEK kinase, Ras protein, and a Gi/o protein. A decrease in expression or activity of one or more components (e.g. as compared to a negative control) indicates that the test agent inhibits the Ras-MEK-ERK 1/2 cascade (pathway) and is expected to show analgesic activity.

When screening for modulators, a positive assay result need not indicate that particular test agent is a good pharmaceutical. Rather a positive test result can simply indicate that the test agent can be used to modulate expression or activity of a member of the Ras-MEK-ERK 1/2 cascade and/or can also serve as a lead compound in the development of other modulators (e.g., inhibitors).

Using known activities, and/or nucleic acid sequences, and/or amino acid sequences of the components of the the Ras-MEK-ERK 1/2 pathway, component expression level(s) and/or activity can readily be determined according to a number of different methods, e.g., as described below. In particular, expression levels of one or more componets of the pathway can be altered by changes in the copy number of the gene(s) encoding those componets, and/or by changes in the transcription of the gene product (i.e. transcription of mRNA), and/or by changes in translation of the gene product (i.e. translation of the protein), and/or by post-translational modification(s) (e.g. protein folding, glycosylation, etc.). Thus useful assays of this invention include assaying for copy number, level of transcribed mRNA, level of translated protein, activity of translated protein, etc. Examples of such approaches are described below.

A) Nucleic-Acid based assays.

1) Target molecules.

Changes in expression level(s) or one or more componets of the Ras-MEK-ERK 1/2 pathway can be detected by measuring changes in mRNA encoding such component(s) and/or a nucleic acid derived from the mRNA (e.g. reverse-transcribed cDNA, etc.). In order to measure the expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism, or from cells in culture. The sample may be of any biological tissue or fluid. Biological samples may also include organs or sections of tissues such as frozen sections taken for histological purposes.

The nucleic acid (e.g., mRNA nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to polymerase chain reaction (PCR, see. e.g, Innis, et al., (1990) PCR Protocols. A guide to Methods and Application. Academic Press, Inc. San Diego,), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Nat. Acad. Sci. USA 86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of Ras-MEK-ERK 1/2 component nucleic acid in a sample, the nucleic acid sample is one in which the concentration of the mRNA transcript(s), or the concentration of the nucleic acids derived from the mRNA transcript(s), is proportional to the transcription level (and therefore expression level) of the gene(s) of interest. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large differences of changes in nucleic acid concentration is desired, no elaborate control or calibration is required.

In the simplest embodiment, the nucleic acid sample is the total mRNA or a total cDNA isolated and/or otherwise derived from a biological sample (e.g. a neurological cell or tissue). The nucleic acid may be isolated from the sample according to any of a number of methods well known to those of skill in the art as indicated above.

2) Hybridization-Based assays.

Using the known sequences for components of the Ras-MEK-ERK 1/2 pathway, detecting and/or quantifying the EG-1 transcript(s) can be routinely accomplished using nucleic acid hybridization techniques (see, e.g., Sambrook et al. supra). For example, one method for evaluating the presence, absence, or quantity of reverse-transcribed cDNA involves a “Southern Blot”. In a Southern Blot, the DNA (e.g., reverse-transcribed mRNA), typically fragmented and separated on an electrophoretic gel, is hybridized to a probe specific for subject nucleic acid(s) (or to a mutant thereof). Comparison of the intensity of the hybridization signal from the probe with a “control” probe (e.g. a probe for a “housekeeping gene) provides an estimate of the relative expression level of the target nucleic acid.

Alternatively, the mRNA of interest can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target EG-1 mRNA. Appropriate controls (e.g. probes to housekeeping genes) provide a reference for evaluating relative expression level.

An alternative means for determining the expression level(s) of various componets of the Ras-MEK-ERK 1/2 pathway is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non- specific hybridization.

3) Amnlirication-Based assays.

In another embodiment, amplification-based assays can be used to measure expression (transcription) level of one or more components of the Ras-MEK-ERK 1/2 pathway. In such amplification-based assays, the target nucleic acid sequences (e.g. ERK nucleic acids, Gi/o nucleic acids etc.) act as template(s) in amplification reaction(s) (e.g. Polymerase Chain Reaction (PCR) or reverse-transcription PCR (RT-PCR)). In a quantitative amplification, the amount of amplification product will be proportional to the amount of template in the original sample. Comparison to appropriate (e.g. tissue or cells unexposed to the test agent) controls provides a measure of the target transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One typical internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of MRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y.. The known nucleic acid sequence(s) for EG-1 are sufficient to enable one of skill to routinely select primers to amplify any portion of the gene.

4) Hybridization Formats and Optimization of Hybridization Conditions.

a) Array-Based Hybridization Formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array based formats are well known to those of skill in the art (see, e.g., Pastinen (1997) Genome Res. 7 : 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No: 5,807,522). This patent describes the use of an automated system that taps a microcapillary against a surface to deposit a small volume of a biological sample. The process is repeated to generate high-density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high-density arrays is also described in U.S. Pat. Nos. 5,744,305, 5,800,992 and 5,445,934.

b) Other Hybridization Formats.

As indicated above a variety of nucleic acid hybridization formats are known to those skilled in the art. For example, common formats include sandwich assays and competition or displacement assays. Such assay formats are generally described in Hames and Higgins (1985) Nucleic Acid Hybridization. A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemi-luminescent agents, enzymes, and antibodies that can serve as specific binding pair members for a labeled ligand.

Detection of a hybridization complex may require the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

c) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.).

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

d) Labeling and Detection of Nucleic Acids.

The probes used herein for detection of one or more componets of the Ras-MEK-ERK 1/2 pathway (e.g. nucleic acids encoding ERK, MEK kinase, Ras protein, a Gi/o protein, etc.) expression levels can be full length or less than the full length of the target nucleic acid. Shorter probes are empirically tested for specificity. Preferred probes are sufficiently long so as to specifically hybridize with the target nucleic acid(s) under stringent conditions. The preferred size range is from about 10, 15, or 20 bases to the length of the target mRNA, more preferably from about 30 bases to the length of the target mRNA, and most preferably from about 40 bases to the length of the target mRNA. The probes are typically labeled, with a detectable label as described above.

B) Detection of Expressed Protein

A) Assay Formats.

In addition to, or in alternative to, the detection of Ras-MEK-ERK 1/2 pathway nucleic acid(s), alterations in expression of components of the Ras-MEK-ERK 1/2 cascade pathway can be detected and/or quantified by detecting and/or quantifying the amount and/or activity of translated Ras-MEK-ERK 1/2 cascade polypeptide(s) (e.g. ERK, MEK kinase, Ras protein, a Gi/o protein, etc.).

The expression of members of the Ras-MEK-ERK 1/2 cascade can be detected and quantified by any of a number of methods well known to those of skill in the art. These can include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one embodiment, the member(s) of the Ras-MEK-ERK 1/2 cascade are detected/quantified in an electrophoretic protein separation (e.g., a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another embodiment, Western blot (immunoblot) analysis is used to detect and quantify the presence of members of the Ras-MEK-ERK 1/2 cascade of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target member, e.g., polypeptide(s), and may be directly labeled or alternatively may be subsequently detected using labeled antibodies (e.g., labeled sheep anti-mouse antibodies) that specifically bind to a domain of the antibody.

In certain embodiments, the members of the Ras-MEK-ERK 1/2 cascade are detected using an immunoassay. As used herein, an immunoassay is an assay that utilizes an antibody to specifically bind to the analyte (e.g., the target polypeptide(s), such as a member of the Ras-MEK-ERK 1/2 cascade). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (e.g., member of the Ras-MEK-ERK 1/2 cascade). In certain embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Typical immunoassays for detecting the target polypeptide(s), e.g., a member of the Ras-MEK-ERK 1/2 cascade, are either competitive or noncompetitive. Noncompetitive immunoassays are assays in which the amount of captured analyte is directly measured. In one “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (a member of the Ras-MEK-ERK 1/2 cascade) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one embodiment, the antibody is immobilized on a solid substrate. The amount of target polypeptide bound to the antibody may be determined either by measuring the amount of target polypeptide present in a polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind a member of the Ras-MEK-ERK 1/2 cascade, either alone or in combination. In the case where the antibody that binds a member of the Ras-MEK-ERK 1/2 cascade is not labeled, a different detectable marker, for example, an enzyme-labeled antibody capable of binding to the monoclonal antibody which binds the member of the Ras-MEK-ERK 1/2 cascade, may be employed. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention may also be other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds a member of the Ras-MEK-ERK 1/2 cascade is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2 M sodium chloride, preferably about that of 0.15 M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents may also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein can be routinely produced as described below.

B) Antibodies to Members of the Ras-MEK-ERK 1/2 Cascade.

Either polyclonal or monoclonal antibodies can be used in the immunoassays of the invention described herein. Polyclonal antibodies are typically raised by multiple injections (e.g. subcutaneous or intramuscular injections) of substantially pure polypeptides or antigenic polypeptides into a suitable non-human mammal. The antigenicity of the target peptides can be determined by conventional techniques to determine the magnitude of the antibody response of an animal that has been immunized with the peptide. Generally, the peptides that are used to raise antibodies for use in the methods of this invention should generally be those which induce production of high titers of antibody with relatively high affinity for target polypeptides, such as a member of the Ras-MEK-ERK 1/2 cascade.

If desired, the immunizing peptide can be coupled to a carrier protein by conjugation using techniques that are well-known in the art. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g. a mouse or a rabbit).

The antibodies are then obtained from blood samples taken from the mammal. The techniques used to develop polyclonal antibodies are known in the art (see, e.g., Methods of Enzymology, “Production of Antisera With Small Doses of Immunogen: Multiple Intradermal Injections ”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see, for example, Coligan, et al. (1991) Unit 9, Current Protocols in Immunology, Wiley Interscience).

In certain embodiments, however, the antibodies produced will be monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′)^{2′}, and/or single-chain antibodies (e.g. scFv) which are capable of binding an epitopic determinant. Also, in this context, the term “mab's of the invention” refers to monoclonal antibodies with specificity for a member of the Ras-MEK-ERK 1/2 cascade.

The general method used for production of hybridomas secreting mAbs is well known (Kohler and Milstein (1975) Nature, 256:495). Briefly, as described by Kohler and Milstein the technique comprised isolating lymphocytes from regional draining lymph nodes of five separate cancer patients with either melanoma, teratocarcinoma or cancer of the cervix, glioma or lung, (where samples were obtained from surgical specimens), pooling the cells, and fusing the cells with SHFP-1. Hybridomas were screened for production of antibody which bound to cancer cell lines. Confirmation of specificity among mab's can be accomplished using relatively routine screening techniques (such as the enzyme-linked immunosorbent assay, or “ELISA”) to determine the elementary reaction pattern of the mAb of interest.

Antibody fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pIII) and the antibody fragment-pill fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20 fold-1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural V_{H} and V_{L} repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1 μM to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

It will also be recognized that antibodies can be prepared by any of a number of commercial services (e.g., Berkeley antibody laboratories, Bethyl Laboratories, Anawa, Eurogenetec, etc.).

C) Assays for Activity

Another aspect of the invention is a method of assaying a compound that modulates (e.g. inhibits) the Ras-MEK-ERK 1/2 cascade, by selecting, as a test agent, a molecule or compound or composition that modulates the activity of a member of the Ras-MEK-ERK 1/2 cascade. Preferably, the agent will inhibit the activity of a member of the Ras-MEK-ERK 1/2 cascade.

The ability of a test compound to inhibit the activity of a member of the Ras-MEK-ERK 1/2 cascade may be determined with suitable assays measuring a member's function. For example, responses such as its activity, e.g., enzymatic activity, phosphorylation activity, or a member's ability to bind its ligand, adapter molecule or substrate may be determined in in vitro assays. Cellular assays can be developed to monitor a modulation of second messenger production, changes in cellular metabolism, changes in intracellular location or effects on enzymatic activity. Immunoassays and nociceptive threshold assays, such as a withdrawal threshold assay, can also be used. These assays may be performed using conventional techniques developed for these purposes.

1) Kinase and Phosphorylation Activity

Some members of the Ras-MEK-ERK 1/2 cascade are kinases, e.g., MEK and ERK 1/2. As a result, an inhibitor of the cascade can be assayed by modulation of the kinase activity and/or phosphorylation of a target molecule. For example, kinase activity can be assayed, e.g., using at least a partially purified kinase, e.g., MEK, in a reconstituted phospholipid environment with radioactive ATP as the phosphate donor and a histone protein or a short peptide as the substrate. See, e.g., T. Kitano, M. Go, U. Kikkawa, Y. Nishizuka, (1986) Meth Enzymol., 124:349-352; and, R. O. Messing, P. J. Peterson, C. J. Henrich, (1991) J. Biol. Chem., 266: 23428-23432. Recent improvements include a rapid, highly sensitive chemiluminescent assay that measures protein kinase activity at physiological concentrations and can be automated and/or used in high-throughput screening. See, e.g., C Lehel, S. Daniel-Issakani, M. Brasseur, B. Strulovici, (1997) Anal. Biochem, 244:340-346. Immunoassays can also be used to detect phosphorylation, e.g., using antibodies specific for phosphorylated (poly)peptides to determined kinase activity. In addition, many other kinase and phosphorylation assays are known to one of skill in the art.

2) Location Assays

Inhibitors that affect the intracellular location of a member of the Ras-MEK-ERK 1/2 cascade can be identified by assays in which the intracellular location of a member is determined. For example, the intracellular location can be determined by, e.g., fractionation or by immunohistochemistry. See, e.g., R. O. Messing, P. J. Peterson, C. J. Henrich, (1991) J. Biol. Chem., 266: 23428-23432; U.S. Pat. No. 5,783,405. This describes assays for intracellular location of PKCε which can be easily adapted by one of skill in the art for members of the Ras-MEK-ERK 1/2 cascade.

3) Nociceptive Threshold Assays

An inhibitor's effect on a member's activity in the Ras-MEK-ERK 1/2 cascade can be measured using nociceptive threshold assays. Mechanical, thermal and chemical nociceptive threshold assays can be used.

For example, mechanical hyperalgesia can be determined by a Randall-Selitto paw-withdrawal test (nociceptive flexion reflex). See, e.g., Randall and Setillo (1957). The nociceptive flexion reflex can be quantified using an Ugo Basile analgesymeter that applies a linearly increasing mechanical force measured in grams to the animal's hindpaw. (Stoelting, Chicago, Ill.). See, e.g., K. O. Aley, J. D. Levine (1997) J. Neuroscience 17: 8018-23; and Taiwo et al., (1989) The contribution of training to sensitivity in the nociceptive paw-withdrawal test, Brain Res., 487:148-151. The analgesymeter is basically a device that exerts a force that increases at a constant rate. The force is applied to the animal's paw, which is placed on a small plinth under a pusher, e.g., a cone-shaped pusher. The operator depresses a switch to start the mechanism that exerts the force. The nociceptive threshold is defined as the force, e.g., in grams, at which the animal withdraws its paw or optionally, vocalizes.

In another mechanical nociceptive threshold assay, the basal mechanical nociceptive threshold is measured as the frequency at which an animal withdraws their paw after being poked in the hind paw with a von Frey hair or filament (VFH; Ainsworth, London, UK). The von Frey hair or filament is applied using a variety of forces, e.g., at intensities of 3.82 N/mm2 (36.3 mN) and 4.54 N/mm² (60.3 mN), e.g., using an up-and-down method (Chaplan et al., (1994) Quantitative assessment of tactile allodynia in the rat paw, J. Neuroscien Meth., 53:55-63; Kinnman & Levine, (1995) Involvement of the sympathetic postganglionic neuron in capsaicin-induced secondary hyperalgesia in the rat, Neuroscience, 65:283-291; and Aley et al., (1996), Vincristine hyperalgesia in the rat: a model of painful vincristine neuropathy in humans, Neuroscience, 73:259-265). See also, Dixon W. J., (1980) Annu Rev Pharmacol Toxicol, 20:441-462. A slightly blunted needle can also be used to touch the plantar surface of the hind paw, which causes a dimpling of the skin without penetrating the skin. Times for withdraw can be measured.

Thermal nociceptive thresholds can be determined by the Hargreave thermal nociceptive test. See, e.g., K. O. Aley, D. B. Reichling, J. D. Levine, (1996) Neuroscience, 73:259-265. Chemically induced hyperalgesia can be determined by a writhing test. See, e.g., S. J. Ward, A. E. Takemori, (1983) J. Pharmacol. Exp. Ther. 224:525-530. The Writhing Assay involves the continuous, chemically-induced pain of visceral origin to an animal, such as a mouse or rat. See, e.g., Gyires et al., Arch. int. Pharmacodyn, 267, 131-140 (1984); C. Vander Wende et al., Fed. Proc., 25, 494 (1956); Koster et al., Fed. Proc., 18, 412 (1959); and Witken et al., J. Pharmacol. Exp. Ther., 133, 400-408 (1961). As a result of the chemical irritation to the animal (e.g., using phenylbenzoquinone (PBQ) or acetic acid), a characteristic stretching and writhing of the animal (dorsiflexion of the animal's back, extension of its hindlimbs and the strong contraction of its abdominal musculature) will generally occur. The intensity of this pain reaction is determined by the number of writhes exhibited by the animal during a given period of time. Inhibitors will reduce the number of writhes of the animal and appear to restore the normal nociceptive threshold of the animal.

D) Assay Optimization.

The assays of this invention have immediate utility in screening for agents that inhibit the Ras-MEK-ERK 1/2 cascade in a cell, tissue or organism. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred isolation conditions), antibody conditions that maximize signal to noise, protocols that improve throughput, etc. In addition, assay formats can be selected and/or optimized according to the availability of equipment and/or reagents. Thus, for example, where commercial antibodies or ELISA kits are available it may be desired to assay protein concentration.

Routine selection and optimization of assay formats is well known to those of ordinary skill in the art.

II. Pre-screening for Agents That Inhibit the Ras-MEK-ERK 1/2 Cascade

In certain embodiments it is desired to pre-screen test agents for the ability to interact with (e.g. specifically bind to) a member of the Ras-MEK-ERK 1/2 cascade and/or to a nucleic acid that encodes such a member. Specifically, binding test agents are likely to interact with and thereby alter a member of the Ras-MEK-ERK 1/2 cascade's expression and/or activity. Thus, in some preferred embodiments, the test agent(s) are pre-screened for binding to a member of the Ras-MEK-ERK 1/2 cascade and/or to a nucleic acid encoding such a member before performing the more complex assays described above.

The test agent can be contacted directly to the member of the Ras-MEK-ERK 1/2 cascade, contacted to a cell containing the Ras-MEK-ERK 1/2 cascade, and/or to a tissue comprising such cells (e.g. to a brain slice preparation, or a galglion prep), and/or contacted to an animal (e.g., a mammal) comprising a Ras-MEK-ERK 1/2 cascade.

Such pre-screening can readily be accomplished with simple binding assays. Means of assaying for specific binding or the binding affinity of a particular ligand for a nucleic acid and/or for a protein are well known to those of skill in the art. In preferred binding assays, the member of the Ras-MEK-ERK 1/2 cascade and/or the nucleic acid(s) encoding such a member, is immobilized and exposed to a test agent (which can be labeled), or alternatively, the test agent(s) are immobilized and exposed to a member of the Ras-MEK-ERK 1/2 cascade (which can be labeled). The immobilized moiety is then washed to remove any unbound material and the bound test agent or bound member of the Ras-MEK-ERK 1/2 cascade is detected (e.g. by detection of a label attached to the bound molecule). The amount of immobilized label is proportional to the degree of binding between the member of the Ras-MEK-ERK 1/2 cascade and the test agent.

In certain embodiments, the detecting is via a method selected from the group consisting of capillary electrophoresis, a Western blot, mass spectroscopy, ELISA, immunochromatography, and immunohistochemistry.

III. Scoring the Assay(s).

The assays of this invention are scored according to standard methods well known to those of skill in the art. The assays of this invention are typically scored as positive where there is a difference between the activity seen with the test agent present or where the test agent has been previously applied, and the (usually negative) control. In certain embodiments, the change is a statistically significant change, e.g. as determined using any statistical test suited for the data set provided (e.g. t-test, analysis of variance (ANOVA), semiparametric techniques, non-parametric techniques (e.g. Wilcoxon Mann-Whitney Test, Wilcoxon Signed Ranks Test, Sign Test, Kruskal-Wallis Test, etc.). Preferably the statistically significant change is significant at least at the 85%, more preferably at least at the 90%, still more preferably at least at the 95%, and most preferably at least at the 98% or 99% confidence level). In certain embodiments, the change is at least a 10% change, preferably at least a 20% change, more preferably at least a 50% change and most preferably at least a 90% change.

IV. Agents for Screening Combinatorial Libraries (e.g., Small Organic Molecules)

Virtually any agent can be screened according to the methods of this invention. Such agents include, but are not limited to nucleic acids, proteins, sugars, polysaccharides, glycoproteins, lipids, and small organic molecules. The term small organic molecules typically refers to molecules of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

Conventionally, new chemical entities with useful properties are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one embodiment, high throughput screening methods involve providing a library containing a large number of potential therapeutic compounds (candidate compounds). Such “combinatorial chemical libraries” are then screened in one or more assays, as described herein to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis by combining a number of chemical “building blocks” such as reagents. For example, a linear combinatorial chemical library such as a polypeptide (e.g., mutein) library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, Dec. 26, 1991), encoded peptides (PCT Publication WO 93/20242, Oct. 14, 1993), random bio-oligomers (PCT Publication WO 92/00091, Jan. 9, 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, Jan 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. Nos. 5,506,337, benzodiazepines 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include, but are not limited to, automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist and the Venture™ platform, an ultra-high-throughput synthesizer that can run between 576 and 9,600 simultaneous reactions from start to finish (see Advanced ChemTech, Inc. Louisville, Ky.)). Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

V. High Throughput Screening

Any of the assays described herein are amenable to high-throughput screening (HTS). Moreover, the cells utilized in the methods of this invention need not be contacted with a single test agent at a time. To the contrary, to facilitate high-throughput screening, a single cell may be contacted by at least two, preferably by at least 5, more preferably by at least 10, and most preferably by at least 20 test compounds. If the cell scores positive, it can be subsequently tested with a subset of the test agents until the agents having the activity are identified.

High throughput assays for hybridizaiton assays, immunoassays, and for various reporter gene products are well known to those of skill in the art. For example, multi-well fluorimeters are commercially available (e.g., from Perkin-Elmer).

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

VI. Modulator Databases.

In certain embodiments, the agents that score positively in the assays described herein (e.g. show an ability to inhibit the expression or activity of a member of the Ras-MEK-ERK 1/2 pathway) can be entered into a database of putative and/or actual inhibitors of the Ras-MEK-ERK 1/2 cascade. The term database refers to a means for recording and retrieving information. In certain embodiments the database also provides means for sorting and/or searching the stored information. The database can comprise any convenient media including, but not limited to, paper systems, card systems, mechanical systems, electronic systems, optical systems, magnetic systems or combinations thereof. Typical databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

VII. Assays for Screening for Inhibitors of Multiple Pain Pathways

The invention also provides for methods for screening for inhibitors of multiple pain pathways (e.g. the Ras-MEK-ERK 1/2 pathway, and/or the PKA pathway, and/or the PKCε pathway). In certain embodiments, the methods involve screening a test agent for activity in the Ras-MEK-ERK 1/2 prior to, simultaneous with, or after screening the test agent for activity in the PKA and/or PKC pathway. Methods of screening for inhibitors of PKCε are described in U.S. Pat. No. 6,376,467. In certain embodiments, the method involves assaying a test agent for the ability to modulate activity of a tetrodotoxin-resistant sodium current wherein inhibition of the tetrodotoxin-resistant sodium current indicates that said test agent inhibits inflammatory or neuropathic pain mediated by PKA cascade, PKCε cascade and Ras-MEK-ERK 1/2 cascade. In certain embodiments, the assaying comprises: contacting a neurological tissue preparation (e.g., a neuronal culture, such as a primary neuronal culture, a dorsal root ganglion preparation and the like) with an agent that induces hyperalgesia (e.g., epinephrine, NGF, bradykinin, norepinephrine, prostaglandin E₂ and the like). The neurological tissue preparation is contacted with the test agent; and is assayed for modulation of the activity of the tetrodotoxin-resistant sodium current.

A) PKA and PKCε Pathaways.

Multiple signaling pathways mediate hyperalgesia produced by inflammatory agents. The inflammatory mediators prostanglin E₂ (PGE₂), serotonin, and adenosine produce hyperalgesia through the activation of protein kinas A (PKA) (see, e.g., Gold et al. (1996), Hyperalgesic agents increase a tetrodotoxin-resistant Na+ current in nociceptors, Proc. Natl. Acad. Sci. USA, 93:1108-1112; Gold et al., (1998), Modulation of TTX-R 1 Na by PKC and PKA and their role in PGE2-induced sensitization of rat sensory neurons in vitro, J. Neurosci. 18:10345-10355; Khasar et al., (1998a), A tetrodotoxin-resistant sodium current mediates inflammatory pain in the rat, Neurosci. Lett., 256:17-20; and, Khasar et al., (1999a), A novel nociceptor signalling pathway revealed in protein kinase Cε mutant mice, Neuron, 24: 253-260. This process is facilitated by nitric oxide. See, e.g., Aley et al., (1998), Nitric Oxide signaling in pain and nociceptor sensitization in the rat, J. Neurosci., 18:7008-7014; and, Chen and Levine, (1999), NOS inhibitor antagonism of PGE2-induced mechanical sensitization of cutaneous C-fiber nociceptors in the rat., J. Neurophysiol., 81:963-966.

Epinephrine also induces hyperalgesia. The direct action of epinephrine on primary sensory afferent neurons is in contrast to other hyperalgesia-inducing agents which indirectly sensitize these neurons. For instance, bradykinin and norepinephrine affect nociceptors by causing intermediary cells to release prostaglandins that act on nociceptors and by causing sympathetic neurons to send signals to nociceptors (Andreev et al. (1995) Pain 63: 109-115; Ferreira et al. (1997) Brit. J. Pharmacol. 121: 883-888; Taiwo et al. (1990) Neuroscience 39: 523-531).

Epinephrine-induced hyperalgesia is a model system for the study of naturally occurring hyperalgesia, and the clinical relevance of this system is supported by the fact that local administration of epinephrine exacerbates symptoms in patients with neuropathic pain (B. Choi, M. C. Rowbotham, (1997) Pain 69:55-63) and that epinephrine causes anginal pain in the absence of apparent ischemia (B. Eriksson et al., (1995) Am. J. Cardiol. 75:241-245). It was previously shown that epinephrine acting through β2-adrenegic receptors on primary afferent nociceptors, produces mechanical hyperalgesia in part through PKA but also through the epsilon isozyme of protein kinase C (PKCε). See, e.g., Khasar et al., (1999a), supra. PKCε also contributes to bradykinin-induced sensitization of nociceptors to heat. See, e.g., Cesare et al., (1999), Specific involvement of PKC-epsilon in sensitization of the neuronal response to painful heat, Neuron 23:617-624. In addition, the PKC family of proteins contributes to diabetic neuropathic hyperalgesia (S. C. Ahlgren, J. D. Levine, J. Neurophys. 72, 684-692 (1994)) and to bradykinin-induced activation and sensitization of nociceptors (S. M. McGuirk, A. C. Dolphin, Neuroscience 49, 117-28 (1992); L. M. Boland, A. C. Allen, R. Dingledine, J. Neurosci. 11, 1140-9 (1991)).

In the present invention, the β-adrenergic receptors bound by epinephrine in turn activate three independent second messenger pathways, the PKC pathway, the cyclic AMP (“cAMP”)/protein kinase A (“PKA”) pathway and the Ras-MEK-ERK1/2 cascade. Although epinephrine-induced hyperalgesia is not mediated by prostaglandins, both epinephrine and prostaglandin E₂ (“PGE₂”), enhance the tetrodotoxin-resistant sodium current (TTX-RINa), which is important in inflammatory mediator-induced hyperalgesia and nociceptor sensitization. TTX-R INa can be a target of PKCε cascade, PKA cascade and the Ras-MEK-ERK 1/2 cascade.

VIII. Sex Hormones and Pain Pathways

Gender and sex-hormone-related differences in pain and nociception have been described, although most of the studies have addressed the modulatory role of sex steroids on CNS mechanism of nociception. See, e.g., Romero, M. T. & Bodnar, R. J. (1986) Gender differences in two forms of coldwater swim analgesia. Physiol. Behav., 37, 893-897; Fillingim, R. B. & Maixner, W. (1995) Gender differences in the responses to noxious stimuli. Pain Forum, 4, 209-221; Unruh, A. M. (1996) Gender variations in clinical pain experience. Pain, 65, 123-167; Pare, W. P. (1969) Age, sex, and strain differences in the aversive threshold to grid shock in the rat. J. Comp Physiol. Psychol, 69, 214-218; Kepler, K. L., Kest, B., Kiefel, J. M., Cooper, M. L. & Bodnar, R. J. (1989) Roles of gender, gonadectomy and estrous phase in the analgesic effects of intracerebroventricular morphine in rats. Pharmacol. Biochem. Behav., 34, 119-127; Aloisi, A. M., Albonetti, M. E. & Carli, G. (1994) Sex differences in the behavioural response to persistent pain in rats. Neurosci. Lett., 179, 79-82; Coyle, D. E., Sehlhorst, C. S. & Mascari, C. (1995) Female rats are more susceptible to the development of neuropathic pain using the partial sciatic nerve ligation (PSNL) model. Neurosci. Lett., 186, 135-138; Beatty, W. W. & Beatty, P. A. (1970) Hormonal determinants of sex differences avoidance behavior and reactivity to electric shock in the rat. J. Comp. Physiol. Psychol, 73, 446-455; Marks, H. E., Fargason, B. D. & Hobbs, S. H. (1972) Reactivity to aversive stimuli as a function of alterations in body weight in normal and gonadectomized female rats. Physiol. Behav, 9, 539-544; Romero, M. T., Cooper, M. L., Komisaruk, B. R. & Bodnar, R. J. (1988) Gender specific and gonadectomy-specific effects upon swim analgesia: role of steroid replacement therapy. Physiol. Behav, 44, 257-265; Baamonde, A. I., Hidalgo, A. & Andres-Trelles, F. (1989) Sex-related differences in the effects of morphine and stress on visceral pain. Neuropharmacology, 28, 967-970; Candido, J., Lutfy, K., Billings, B., Sierra, V., Duttaroy, A., Inturrisi, C. E. & Yobum, B. C. (1992) Effect of adrenal and sex hormones on opioid analgesia and opioid receptor regulation. Pharmacol. Biochem. Behav, 42, 685-692; and, Dawson-Basoa, M. B. & Gintzler, A. R. (1993) 17-Beta-estradiol and progesterone modulate an intrinsic opioid analgesic system. Brain Res., 601, 241-245. This invention pertains to the identification of gender differences in the signaling of pain, e.g., inflammatory pain and/or neuropathic pain, at the level of primary afferent nociceptors.

The Ras-MEK-ERK 1/2 cascade identified herein and present in peripheral nociceptors contributes to inflammatory pain (& neuropathic pain) along with protein kinase A (PKA) and protein kinase C epsilon (PKCε) in males, but contributes to a much greater extent in females. Drugs that inhibit this pathway represent a novel class of analgesics. This class of analgesics is particularly well suited for, e.g., inflammatory pain, which is much more prevalent in women (e.g. rheumatoid arthritis, systemic lupus etc).

In addition, sex-hormones can be used as analgesics to inhibit pain signalling pathways, e.g., PKA cascade and PKCε cascade, because PKCε, PKA and nitric oxide (NO) signalling pathways were found to contribute to epinephrine-induced hyperalgesia in males but not in females, due to suppression by female sex-hormones, e.g., oestrogen. The Ras-MEK-ERK 1/2 cascade does not appear to be suppressed by sex-hormones, e.g., oestrogen.

Thus, in one embodiment, the invention provides a method of decreasing hyperalgesia or pain, e.g., inflammatory pain, neuropathic pain, pain of a type produced by formalin, and the like, in a mammal (e.g., male or female), said method comprising administering estrogen or an estrogen analog or agonist to said mammal in a concentration sufficient to inhibit contributions of P adrenegic receptor, e.g., β2 adrenegic receptor, mediated PKA or PKCε to pain signaling. The estrogen analog or agonist includes but is not limited to, e.g., an estradiol, an estrone, an ethinyl estradiol, a diethylstilbestrol, a mestranol, an estrone, a conjugated estrogen, a chlorotrianisene and analogs thereof.

As described herein, combinations of inhibitors can be used. For example, the methods of decreasing hyperalgesia or pain also comprise administering an inhibitor of a Ras-MEK-ERK 1/2 cascade to said mammal in a concentration sufficient to inhibit the Ras-MEK-ERK 1/2 cascade along with the estrogen or an estrogen analog or agonist. In still another embodiment, the methods further comprise administering an inhibitor of a prostaglandin E₂ cascade to said mammal in a concentration sufficient to inhibit prostaglandin E₂ hyperalgesia along with estrogen or estrogen analog or agonist.

IX. Modulating Activity of a Ras-MEK-ERK 1/2 Cascade

In certain embodiments, this invention contemplates the use of Ras-MEK-ERK 1/2 cascade targeted therapeutics in the treatment of pain or symptoms associated with acute or chronic pain. Typically such methods will entail administration of an agent that modulates (e.g. downregulates) activity of the Ras-MEK-ERK 1/2 cascade, e.g. by inhibiting transcription, and/or translation, and/or activity of one or more components of the Ras-MEK-ERK 1/2 pathway (e.g. ERK 1/2, MEK kinase, Ras, a Gi/o protein, etc.). Such agents include, but are not limited to agents identified according to the screening methods described herein.

Other agents can also be used to downregulate expression of Ras-MEK-ERK 1/2 cascade. Such agents can include, but are not limited to antisense molecules, Ras-MEK-ERK 1/2 cascade specific riibozymes, Ras-MEK-ERK 1/2 cascade specific catalytic DNAs, Ras-MEK-ERK 1/2 cascade-specific RNAi, intrabodies directed against Ras-MEK-ERK 1/2 cascade proteins, and “gene therapy” approaches that knock out Ras-MEK-ERK 1/2 cascade.

A) Antisense Approaches.

Gene expression of one or members of the Ras-MEK-ERK 1/2 pathway can be downregulated or entirely inhibited by the use of antisense molecules. An “antisense sequence or antisense nucleic acid” is a nucleic acid that is complementary to a nucleic acid (e.g. mRNA) coding a member of the Ras-MEK-ERK 1/2 pathway or a subsequence thereof. Binding of the antisense molecule to the mRNA interferes with normal translation of the Ras-MEK-ERK 1/2 pathway member.

Thus, in accordance with certain embodiments of this invention, antisense molecules include oligonucleotides and oligonucleotide analogs that are hybridizable mRNA(s) encoding one or more components of the Ras-MEK-ERK 1/2 pathway. This relationship is commonly denominated as “antisense.” The oligonucleotides and oligonucleotide analogs are able to inhibit the function of the RNA, either its translation into protein, its translocation into the cytoplasm, or any other activity necessary to its overall biological function. The failure of the messenger RNA to perform all or part of its function results in a reduction or complete inhibition of expression of Ras-MEK-ERK 1/2 pathway polypeptides.

In the context of this invention, the term “oligonucleotide” refers to a polynucleotide formed from naturally-occurring bases and/or cyclofuranosyl groups joined by native phosphodiester bonds. This term effectively refers to naturally-occurring species or synthetic species formed from naturally-occurring subunits or their close homologs. The term “oligonucleotide” may also refer to moieties which function similarly to oligonucleotides, but which have non naturally-occuring portions. Thus, oligonucleotides may have altered sugar moieties or inter-sugar linkages. Exemplary among these are the phosphorothioate and other sulfur containing species that are known for use in the art. In accordance with some preferred embodiments, at least one of the phosphodiester bonds of the oligonucleotide has been substituted with a structure which functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention.

In one embodiment, the internucleotide phosphodiester linkage is replaced with a peptide linkage. Such peptide nucleic acids tend to show improved stability, penetrate the cell more easily, and show enhances affinity for their target. Methods of making peptide nucleic acids are known to those of skill in the art (see, e.g., U.S. Pat. Nos.: 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides may also include species that include at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH₃, F, OCH₃, OCN, O(CH₂)[n]NH₂ or O(CH₂)[n]CH₃, where n is from 1 to about 10, and other substituents having similar properties.

Such oligonucleotides are best described as being functionally interchangeable with natural oligonucleotides or synthesized oligonucleotides along natural lines, but which have one or more differences from natural structure. All such analogs are comprehended by this invention so long as they function effectively to hybridize with messenger RNA of a member of the Ras-MEK-ERK 1/2 pathway to inhibit the function of that RNA.

The oligonucleotides in accordance with certain embodiments of this invention comprise from about 3 to about 50 subunits. It is more preferred that such oligonucleotides and analogs comprise from about 8 to about 25 subunits and still more preferred to have from about 12 to about 20 subunits. As will be appreciated, a subunit is a base and sugar combination suitably bound to adjacent subunits through phosphodiester or other bonds. The oligonucleotides used in accordance with this invention can be conveniently and routinely made through the well-known technique of solid phase synthesis. Equipment for such syntheses is sold by several vendors (e.g. Applied Biosystems). Any other means for such synthesis may also be employed, however, the actual synthesis of the oligonucleotides is well within the talents of the routineer. It is also will known to prepare other oligonucleotide such as phosphorothioates and alkylated derivatives.

B) Catalytic RNAs and DNAs

1) Ribozymes.

In another approach, expression of a member of the Ras-MEK-ERK 1/2 pathway can be inhibited by the use of ribozymes. As used herein, “ribozymes” include RNA molecules that contain antisense sequences for specific recognition, and an RNA-cleaving enzymatic activity. The catalytic strand cleaves a specific site in a target RNA, preferably at greater than stoichiometric concentration. Two “types” of ribozymes are particularly useful in this invention, the hammerhead ribozyme (Rossi et al. (1991) Pharmac. Ther. 50: 245-254) and the hairpin ribozyme (Hampel et al. (1990) Nucl. Acids Res. 18: 299-304, and U.S. Pat. No. 5,254,678).

Because both hammerhead and hairpin ribozymes are catalytic molecules having antisense and endoribonucleotidase activity, ribozyme technology has emerged as a potentially powerful extension of the antisense approach to gene inactivation. The ribozymes of the invention typically consist of RNA, but such ribozymes may also be composed of nucleic acid molecules comprising chimeric nucleic acid sequences (such as DNA/RNA sequences) and/or nucleic acid analogs (e.g., phosphorothioates).

Accordingly, within one aspect of the present invention ribozymes have the ability to inhibit expression of a member of the Ras-MEK-ERK 1/2 pathway. Such ribozymes may be in the form of a “hammerhead” (for example, as described by Forster and Symons (1987) Cell 48: 211-220,; Haseloff and Gerlach (1988) Nature 328: 596-600; Walbot and Bruening (1988) Nature 334: 196; Haseloff and Gerlach (1988) Nature 334: 585) or a “hairpin” (see, e.g. U.S. Pat. No. 5,254,678 and Hampel et al., European Patent Publication No. 0 360 257, published Mar. 26, 1990), and have the ability to specifically target, cleave an Ras-MEK-ERK 1/2 pathway nucleic acid.

The ribozymes for this invention, as well as DNA encoding such ribozymes and other suitable nucleic acid molecules can be chemically synthesized using methods well known in the art for the synthesis of nucleic acid molecules. Alternatively, Promega, Madison, Wis., USA, provides a series of protocols suitable for the production of RNA molecules such as ribozymes. The ribozymes also can be prepared from a DNA molecule or other nucleic acid molecule (which, upon transcription, yields an RNA molecule) operably linked to an RNA polymerase promoter, e.g., the promoter for T7 RNA polymerase or SP6 RNA polymerase. Such a construct may be referred to as a vector. Accordingly, also provided by this invention are nucleic acid molecules, e.g., DNA or cDNA, coding for the ribozymes of this invention. When the vector also contains an RNA polymerase promoter operably linked to the DNA molecule, the ribozyme can be produced in vitro upon incubation with the RNA polymerase and appropriate nucleotides. In a separate embodiment, the DNA may be inserted into an expression cassette (see, e.g., Cotten and Birnstiel (1989) EMBO J 8(12):3861-3866; Hempel et al. (1989) Biochem. 28: 4929-4933, etc.).

After synthesis, the ribozyme can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

The ribozyme molecule also can be in a host prokaryotic or eukaryotic cell in culture or in the cells of an organism/patient. Appropriate prokaryotic and eukaryotic cells can be transfected with an appropriate transfer vector containing the DNA molecule encoding a ribozyme of this invention. Alternatively, the ribozyme molecule, including nucleic acid molecules encoding the ribozyme, may be introduced into the host cell using traditional methods such as transformation using calcium phosphate precipitation (Dubensky et al. (1984) Proc. Natl. Acad. Sci., USA, 81: 7529-7533), direct microinjection of such nucleic acid molecules into intact target cells (Acsadi et al. (1991) Nature 352: 815-818), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al. (1990) Proc. Natl. Acad. Sci., USA, 89 :6094), lipofection (Felgner et al. (1989) Proc. Natl. Acad. Sci. USA 84: 7413-7417), microprojectile bombardment (Williams et al. (1991) Proc. Natl. Acad. Sci., USA, 88: 2726-2730), polycation compounds such as polylysine, receptor specific ligands, liposomes entrapping the nucleic acid molecules, spheroplast fusion whereby E coli containing the nucleic acid molecules are stripped of their outer cell walls and fused to animal cells using polyethylene glycol, viral transduction, (Cline et al., (1985) Pharmac. Ther. 29: 69; and Friedmann et al. (1989) Science 244: 1275), and DNA ligand (Wu et al (1989) J. Biol. Chem. 264: 16985-16987), as well as psoralen inactivated viruses such as Sendai or Adenovirus. In one preferred embodiment, the ribozyme is introduced into the host cell utilizing a lipid, a liposome or a retroviral vector.

When the DNA molecule is operatively linked to a promoter for RNA transcription, the RNA can be produced in the host cell when the host cell is grown under suitable conditions favoring transcription of the DNA molecule. The vector can be, but is not limited to, a plasmid, a virus, a retrotransposon or a cosmid. Examples of such vectors are disclosed in U.S. Pat. No. 5,166,320. Other representative vectors include, but are not limited to adenoviral vectors (e.g., WO 94/26914, WO 93/9191; Kolls et al. (1994) PNAS 91(1):215-219; Kass-Eisler et al., (1993) Proc. Natl. Acad. Sci., USA, 90(24): 11498-502, Guzman et al. (1993) Circulation 88(6): 2838-48, 1993; Guzman et al. (1993) Cir. Res. 73(6):1202-1207, 1993; Zabner et al. (1993) Cell 75(2): 207-216; Li et al. (1993) Hum Gene Ther. 4(4): 403-409; Caillaud et al. (1993) Eur. J Neurosci. 5(10): 1287-1291), adeno-associated vector type 1 (“AAV-1”) or adeno-associated vector type 2 (“AAV-2”) (see WO 95/13365; Flotte et al. (1993) Proc. Natl. Acad. Sci., USA, 90(22):10613-10617), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick and Burck (1991) Gene Therapy: Application of Molecular Biology, Elsevier Science Publishing Co., Inc., New York, N.Y., and Kreigler (1990) Gene Transfer and Expression: A Laboratory Manual, W. H. Freeman and Company, New York.

To produce ribozymes in vivo utilizing vectors, the nucleotide sequences coding for ribozymes are preferably placed under the control of a strong promoter such as the lac, SV40 late, SV40 early, or lambda promoters. Ribozymes are then produced directly from the transfer vector in vivo

2) Catalytic DNA

In a manner analogous to ribozymes, DNAs are also capable of demonstrating catalytic (e.g. nuclease) activity. While no such naturally-occurring DNAs are known, highly catalytic species have been developed by directed evolution and selection. Beginning with a population of 10¹⁴ DNAs containing 50 random nucleotides, successive rounds of selective amplification, enriched for individuals that best promote the Pb²⁺-dependent cleavage of a target ribonucleoside 3′-O—P bond embedded within an otherwise all-DNA sequence. By the fifth round, the population as a whole carried out this reaction at a rate of 0.2 min⁻¹. Based on the sequence of 20 individuals isolated from this population, a simplified version of the catalytic domain that operates in an intermolecular context with a turnover rate of 1 min⁻¹ (see, e.g., Breaker and Joyce (1994) Chem Biol 4: 223-229.

In later work, using a similar strategy, a DNA enzyme was made that could cleave almost any targeted RNA substrate under simulated physiological conditions. The enzyme is comprised of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (kcat/Km) of approximately 10⁹ M⁻¹min⁻¹ under multiple turnover conditions, exceeding that of any other known nucleic acid enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to Ras-MEK-ERK 1/2 cascade mRNA thereby acting like a ribozyme.

C) RNAi Inhibition of Ras-MEK-ERK 1/2 Cascade Expression.

Post-transcriptional gene silencing (PTGS) or RNA interference (RNAi) refers to a mechanism by which double-stranded (sense strand) RNA (dsRNA) specifically blocks expression of its homologous gene when injected, or otherwise introduced into cells. The discovery of this incidence came with the observation that injection of antisense or sense RNA strands into Caenorhabditis elegans cells resulted in gene-specific inactivation (Guo and Kempheus (1995) Cell 81: 611-620). While gene inactivation by the antisense strand was expected, gene silencing by the sense strand came as a surprise. Adding to the surprise was the finding that this gene-specific inactivation actually came from trace amounts of contaminating dsRNA (Fire et al. (1998) Nature 391: 806-811).

Since then, this mode of post-transcriptional gene silencing has been tied to a wide variety of organisms: plants, flies, trypanosomes, planaria, hydra, zebrafish, and mice (Zamore et al. (2000). Cell 101: 25-33; Gura (2000) Nature 404: 804-808). RNAi activity has been associated with functions as disparate as transposon-silencing, anti-viral defense mechanisms, and gene regulation (Grant (1999) Cell 96: 303-306).

By injecting dsRNA into tissues, one can inactivate specific genes not only in those tissues, but also during various stages of development. This is in contrast to tissue-specific knockouts or tissue-specific dominant-negative gene expressions, which do not allow for gene silencing during various stages of the developmental process (Gura (2000) Nature, 404:804-808). The double-stranded RNA is cut by a nuclease activity into 21-23 nucleotide fragments. These fragments, in turn, target the homologous region of their corresponding mRNA, hybridize, and result in a double-stranded substrate for a nuclease that degrades it into fragments of the same size (Hammond et al. (2000) Nature, 404:293-298; Zamore et al. (2000), Cell 101: 25-33).

Double stranded RNA (dsRNA) can be introduced into cells by any of a wide variety of means. Such methods include, but are not limited to lipid-mediated transfection (e.g. using reagents such as lipofectamine), liposome delivery, dendrimer-mediated transfection, and gene transfer using a viral or bacterial vector. Where the vector expresses (transcribes) a single-stranded RNA, the vector can be designed to trasnscribe two complementary RNA strands that will then hybridize to form a double-stranded RNA.

D) Intrabodies.

In still another embodiment, expression or activity of a member of the Ras-MEK-ERK 1/2 pathway can be inhibited by transfecting the subject cell(s) with a nucleic acid construct that expresses an intrabody. An intrabody is an intracellular antibody, in this case, capable of recognizing and binding to a Ras-MEK-ERK 1/2 pathway polypeptide. The intrabody is expressed by an “antibody cassette”, containing a sufficient number of nucleotides coding for the portion of an antibody capable of binding to the target (Ras-MEK-ERK 1/2 pathway polypeptide) operably linked to a promoter that will permit expression of the antibody in the cell(s) of interest. The construct encoding the intrabody is delivered to the cell where the antibody is expressed intracellularly and binds to the target member(s) of the Ras-MEK-ERK 1/2 pathway, thereby disrupting the target from its normal action. This antibody is sometimes referred to as an “intrabody”.

In one preferred embodiment, the “intrabody gene” (antibody) of the antibody cassette would utilize a cDNA, encoding heavy chain variable (VH) and light chain variable (V_{L}) domains of an antibody which can be connected at the DNA level by an appropriate oligonucleotide as a bridge of the two variable domains, which on translation, form a single peptide (referred to as a single chain variable fragment, “sFv”) capable of binding to a target such as an Ras-MEK-ERK 1/2 cascade protein. The intrabody gene preferably does not encode an operable secretory sequence and thus the expressed antibody remains within the cell.

Anti-Ras-MEK-ERK 1/2 pathway antibodies suitable for use/expression as intrabodies in the methods of this invention can be readily produced by a variety of methods. Such methods include, but are not limited to, traditional methods of raising “whole” polyclonal antibodies, which can be modified to form single chain antibodies, or screening of, e.g. phage display libraries to select for antibodies showing high specificity and/or avidity for member(s) of the Ras-MEK-ERK 1/2 pathway.

The antibody cassette is delivered to the cell by any of the known means. One preferred delivery system is described in U.S. Pat. No. 6,004,940. Methods of making and using intrabodies are described in detail in U.S. Pat. Nos. 6,072,036, 6,004,940, and 5,965,371.

E) Small Organic Molecules.

In still another embodiment, expression and/or protein activity of member of the Ras-MEK-ERK 1/2 cascade can be inhibited by the use of small organic molecules. Such molecules include, but are not limited to molecules that bind to and/or compete with a member of the Ras-MEK-ERK 1/2 cascade. Small organic molecules effective at inhibiting a member of the Ras-MEK-ERK 1/2 cascade expression can be identified with routine screening using the methods described herein.

The methods of inhibiting expression described above are meant to be illustrative and not limiting. In view of the teachings provided herein, other methods of inhibiting a member of the Ras-MEK-ERK 1/2 cascade will be known to those of skill in the art.

X. Compositions Comprising Modulators (e.g. Inhibitors) of Ras-MEK-ERK 1/2 Pathway.

A) Reducing Pain and/or Associated Symptoms.

In certain embodiments, this invention provides compositions that modulate (e.g. inhibit) activity of a Ras-MEK-ERK1/2 pathway. Also provided are methods of use of such compositions. Such compositions can be administered to a subject in need thereof (e.g. a subject experiencing chronic or acute inflammatory or neuropathic pain) to mitigate the experience of pain. The compositions can be administered alone, or in combination with one or more pain-reducing (analgesic) agent(s) that act at a different point in pain perception/signalling process. One class of analgesics, such as NSAIDs (e.g., aspirin, acetaminophen, ibuprofen, indomethacin and the like), down-regulates the chemical messengers of the stimuli that are detected by the nociceptors and another class of drugs, such as opioids, alters the processing of nociceptive information in the CNS. Other analgesics are local anesthetics, anticonvulsants, and antidepressants. Administering one or more classes of drug in addition to Ras-MEK-ERK 1/2 cascade inhibitors can provide more effective amelioration of pain.

The Ras-MEK-ERK 1/2 cascade, PKA cascade and the PKCε cascade are secondary messengers of epinephrine-induced hyperalgesia. The pain associated with this type of hyperalgesia can be addressed by inhibiting all three cascades. Thus, in one embodiment, a composition includes an inhibitor of Ras-MEK-ERK 1/2 cascade along with an inhibitor of the PKA cascade and/or PKCε cascade and a method of administering such a composition as described herein.

The invention also provides methods for desensitizing nocioceptors by inhibiting the Ras-MEK-ERK1/2 cascade. In certain embodiments, the inhibiting comprises inhibiting, e.g., MEK kinase expression or activity, ERK expression or activation, Ras expression or activity, Gi/o expression or activity, β-adrenergic receptor mediated expression or activation of ERK, NGF-mediated expression or activation of ERK, bradykinin-mediated expression or activation of ERK and the like as described above.

In certain embodiments of the invention, the treatment of reducing or lessening pain involves the subject (e.g., the patient) having inflammatory pain. Such inflammatory pain may be acute or chronic and can be due to any number of conditions characterized by inflammation including, without limitation, sunburn, rheumatoid arthritis, osteoarthritis, colitis, carditis, dermatitis, myositis, neuritis and collagen vascular diseases. Administration of a Ras-MEK-ERK 1/2 cascade inhibitor to a subject immediately prior to, during or after an inflammatory event can ameliorate both the acute pain and the chronic hyperalgesia that the subject would otherwise experience.

In another embodiment, the treatment of reducing or lessening pain involves the subject (e.g., the patient) having neuropathic pain. Such subjects can have a neuropathy classified as a radiculopathy, mononeuropathy, mononeuropathy multiplex, polyneuropathy or plexopathy. Diseases in these classes can be caused by a variety of nerve-damaging conditions or procedures, including, without limitation, trauma, stroke, demyelinating diseases, abscess, surgery, amputation, inflammatory diseases of the nerves, causalgia, diabetes, collagen vascular diseases, trigeminal neuralgia, rheumatoid arthritis, toxins, cancer (which can cause direct or remote (e.g. paraneoplastic) nerve damage), chronic alcoholism, herpes infection, AIDS, and chemotherapy. Nerve damage causing hyperalgesia can be in peripheral or CNS nerves.

B) Subject

In certain embodiments, the subject is human. In another embodiment, the subject is a non-human mammal (e.g., a primate, a mouse, a pig, a cow, a cat, a goat, a rabbit, a rat, a guinea pig, a hamster, a horse, a sheep, a dog, a cat and the like). The subject can be male or female, adult, adolescent, or infant.

C) Modes of Administration

In certain embodiments, the methods of this invention involve administering one or more modulators (e.g. inhibitors) of the Ras-MEK-ERK 1/2 cascade to a cell, tissue, or organism, to inhibit pain or one or more symptoms associated with acute or chronic pain. Various inhibitors may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience. See also Remington: The Science and Practice of Pharmacy, 19th ed., (Mack Publishing, 1995).

The inhibitors of the Ras-MEK-ERK 1/2 pathway can be administered alone, or in conjunction with other analgesics (e.g. NSAIDs, inhibitors of a PKA pathway, inhibitors of a PKC pathway, and the like). Numerous analgesics are know to those of skill in the art. Thus, for example, NSAIDs are well known as are modulators of PKC pathway (see, e.g., U.S. Pat. No. 6,376,467).

The inhibitors and various derivatives and/or formulations thereof are useful for parenteral, topical, oral, or local administration, such as by aerosol or transdermally, for prophylactic and/or therapeutic treatment of pain. See also section F. Therapeutic/Prophylatic compositions, herein. The Ras-MEK-ERK 1/2 cascade inhibitors and various derivatives and/or formulations thereof are typically combined with a pharmaceutically acceptable carrier (excipient) to form a pharmacological composition.

The concentration of active agent(s) in the formulation can vary widely, and will be selected primarily based on fluid volumes, viscosities, body weight and the like in accordance with the particular mode of administration selected and the patient's needs. See also section G. Effective Dosages and F. Toxicity, herein. Typically, the active agent(s) are administered in an amount sufficient to alter expression or activity of a member of the Ras-MEK-ERK 1/2 cascade, i.e., an “effective amount”. Single or multiple administrations of the compositions may be administered depending on the dosage and frequency as required and tolerated by the organism or cell or tissue system. In any event, the composition should provide a sufficient quantity of the active agents of this invention to effectively alter expression or activity of a member of the Ras-MEK-ERK 1/2 cascade and preferably to inhibit pain.

D) Ras-MEK-ERK1/2 Pathway Inhibitors

The inhibitors of the invention can act directly on a member of the Ras-MEK-ERK 1/2 cascade or can act indirectly on a member of the Ras-MEK-ERK 1/2 cascade. In certain embodiments, the inhibitor is membrane-permeable. In still another embodiment, the inhibitor inhibits catalytic activity of a member of the Ras-MEK-ERK 1/2 cascade or the inhibitor inhibits intracellular translocation of a member of the Ras-MEK-ERK 1/2 cascade.

Besides obtaining inhibitors to the Ras-MEK-ERK 1/2 cascade with screening methods provided herein, known inhibitors can be used, which include but are not limited to, e.g., an inhibitor of a β2 adrenergic receptor, e.g., an inverse agonist, such as ICI 118,551 or an antagonist, such as propanolol, an inhibitor of Gi/o protein activity, e.g., an isoprenylation inhibitor, pertussis toxin, perillic acid and the like, an inhibitor of Ras activity, e.g., farnesyltransferase, FTase I and the like, an inhibitor of MEK activity, e.g., U0126, PD98059 and the like, and an inhibitor of ERK 1/2 activity.

Inhibitors of the Ras-MEK-ERK 1/2 cascade can be combined with other compounds and/or administered to a subject. For example, compounds include an inhibitor of cAMP, a nonsteroidal anti-inflammatory drug, a local anesthetic, an anticonvulsant, an antidepressant, and an opiod. In another embodiment, an inhibitor of a prostaglandin E₂ cascade can be used, e.g., a nitric oxide synthetase (NOS) inhibitor, such as NG-methyl-L-arginine (L-MNA). In still another embodiment, an inhibitor of a protein kinase A (PKA) cascade and/or protein kinase Cε (PKCε) cascade can be used.

For example, inhibitors of the PKA cascade include, e.g., a Walsh inhibitor peptide (WIPTIDE), a H89 and the like. Inhibitors of PKCε include, e.g., U.S. Pat. No. 5,783,405, which describes a large number of peptides that inhibit PKC isozymes. Of these, the εV1-1, εV1-2, εV1-3, εV1-4, εV1-5 and εV1-6 peptides are selective for PKCε and are preferred peptide inhibitors. Peptide εV1-2 is a particularly preferred inhibitory peptide. Small molecule inhibitors of PKC are described in U.S. Pat. Nos. 5,141,957, 5,204,370, 5,216,014, 5,270,310, 5,292,737, 5,344,841, 5,360,818, and 5,432,198. These molecules belong to the following classes: N,N′-Bis-(sulfonamido)-2-amino-4-iminonaphthalen-1-ones; N,N′-Bis-(amido)-2-amino-4-iminonaphthalen-1-ones; vicinal-substituted carbocyclics; 1,3-dioxane derivatives; 1,4-Bis-(amino-hydroxyalkylamino)-anthraquinones; furo-coumarinsulfonamides; Bis-(hydroxyalkylamino)-anthraquinones; and N-aminoalkyl amides.

E. Anti-Inflammatory Agents

The method of the present invention provides methods of reducing or lessening pain by using an inhibitor of the Ras-MEK-ERK1/2 cascade, and/or PKA cascade, and/or the PKCε cascade comprising administration of the composition of the present invention in conjunction with other treatment agents.

Anti-inflammatory agents have exhibited success in treatment of inflammatory and are now a common and a standard treatment for such disorder so as to reduce inflammatory pain. Any anti-inflammatory agent well-known to one of skill in the art can be used in the methods of the invention. Non-limiting examples of anti-inflammatory agents include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

F. Therapeutic/Prophylactic Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a therapeutically effective amount of a therapeutic agent disclosed herein or a combination of the agent and a pharmaceutically acceptable carrier. Preferably, compositions of the invention comprise a therapeutically effective amount of an inhibitor for the Ras-MEK-ERK1/2 cascade, and/or PKA cascade, and/or the PKCε cascade, and a pharmaceutically acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

Other physiologically acceptable compounds include dispersing agents or preservatives which are particularly useful for preventing the growth or action of microorganism. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well known sterilization techniques.

Generally, the ingredients of the compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Pharmaceutical compositions comprising the inhibitors for the Ras-MEK-ERK1/2 cascade, and/or PKA cascade, and/or the PKCε cascade of the invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the molecules into preparations which can be used pharmaceutically. Proper formulation is dependent upon the route of administration chosen.

For topical or transdermal administration, the inhibitors for the Ras-MEK-ERK1/2 cascade, and/or PKA cascade, and/or the PKCε cascade of the invention may be formulated as solutions, gels, ointments, creams, lotion, emulsion, suspensions, etc. as are well-known in the art. Systemic formulations include those designed for administration by injection, e.g. subcutaneous, intravenous, intramuscular, intrathecal or intraperitoneal injection, as well as those designed for transdermal, transmucosal, inhalation, oral or pulmonary administration. For injection, the inhibitors for the Ras-MEK-ERK1/2 cascade, and/or PKA cascade, and/or the PKCε cascade of the invention may be formulated in aqueous solutions, preferably in physiologically compatible buffers such as Hanks's solution, Ringer's solution, or physiological saline buffer. The solution may contain formulatory agents such as suspending, stabilizing and/or dispersing agents. Alternatively, compositions comprising the inhibitors for the Ras-MEK-ERK1/2 cascade, and/or PKA cascade, and/or the PKCε cascade may be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the inhibitors for the Ras-MEK-ERK1/2 cascade, and/or PKA cascade, and/or the PKCε cascade can be readily formulated by combining the inhibitors for the Ras-MEK-ERK1/2 cascade, and/or PKA cascade, and/or the PKCε cascade with pharmaceutically acceptable carriers well known in the art. Such carriers enable the inhibitors for the Ras-MEK-ERK1/2 cascade, and/or PKA cascade, and/or the PKCε cascade of the invention to be formulated as tablets, pills, dragees, capsules, liquids, gels, syrups, slurries, suspensions and the like, for oral ingestion by a patient to be treated. For oral solid formulations such as, for example, powders, capsules and tablets, suitable excipients include fillers such as sugars, e.g. lactose, sucrose, mannitol and sorbitol; cellulose preparations such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP); granulating agents; and binding agents. If desired, disintegrating agents may be added, such as the cross-linked polyvinylpyrrolidone, agar, or alginic acid or a salt thereof such as sodium alginate.

If desired, solid dosage forms may be sugar-coated or enteric-coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the inhibitors for the Ras-MEK-ERK1/2 cascade, and/or PKA cascade, and/or the PKCε cascade may take the form of tablets, lozenges, etc. formulated in conventional manner.

For administration by inhalation, the inhibitors for the Ras-MEK-ERK1/2 cascade, and/or PKA cascade, and/or the PKCε cascade for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin for use in an inhaler or insufflator may be formulated containing a powder mix of the inhibitors for the Ras-MEK-ERK1/2 cascade, and/orPKA cascade, and/or the PKCε cascade and a suitable powder base such as lactose or starch.

The inhibitors for the Ras-MEK-ERK1/2 cascade, and/or PKA cascade, and/or the PKCε cascade may also be formulated in rectal or vaginal compositions such as suppositories or retention enemas, e.g, containing conventional suppository bases such as cocoa butter or other glycerides.

In addition to the formulations described previously, the inhibitors for the Ras-MEK-ERK1/2 cascade, and/or PKA cascade, and/or or the PKCε cascade may also be formulated as a depot preparation. Such long acting formulations may be administered by implantation (for example subcutaneously or intramuscularly) or by intramuscular injection. Thus, for example, the inhibitors for the Ras-MEK-ERK1/2 cascade, and/or PKA cascade, and/or the PKCε cascade may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver inhibitors for the Ras-MEK-ERK1/2 cascade, and/or PKA cascade, and/or the PKCε cascade of the invention. Certain organic solvents such as dimethylsulfoxide also may be employed, although usually at the cost of greater toxicity. Additionally, the inhibitors for the Ras-MEK-ERK1/2 cascade, and/or PKA cascade, and/or the PKCε cascade may be delivered using a sustained-release system, such as semipermeable matrices of solid polymers containing the therapeutic agent. Various sustained-release materials have been established and are well known by those skilled in the art. Sustained-release capsules may, depending on their chemical nature, release the inhibitors for the Ras-MEK-ERK1/2 cascade, and/or PKA cascade, and/or the PKCε cascade for a few weeks up to over 100 days. Depending on the chemical nature and the biological stability of the inhibitors for the Ras-MEK-ERK1/2 cascade, and/or PKA cascade, and/or the PKCε cascade, additional strategies for stabilization may be employed.

As the inhibitors for the Ras-MEK-ERK1/2 cascade, and/or PKA cascade, and/or the PKCε cascade of the invention may contain charged side chains or termini, they may be included in any of the above-described formulations as the free acids or bases or as pharmaceutically acceptable salts. Pharmaceutically acceptable salts are those salts which substantially retain the biological activity of the free bases and which are prepared by reaction with inorganic acids. Pharmaceutical salts tend to be more soluble in aqueous and other protic solvents than are the corresponding free base forms.

G. Effective Dosages

The inhibitors for the Ras-MEK-ERK1/2 cascade, and/or PKA cascade, and/or the PKCε cascade of the invention will generally be used in an amount effective to achieve the intended purpose. For use to treat hyperalgesia, neuropathic pain, and inflammatory pain, the inhibitors for the Ras-MEK-ERK1/2 cascade, and/or PKA cascade, and/or the PKCε cascade of the invention, or pharmaceutical compositions thereof, are administered or applied in a therapeutically effective amount. A therapeutically effective amount is an amount effective to ameliorate or alleviate pain from the patient being treated. Determination of a therapeutically effective amount is well within the capabilities of those skilled in the art, especially in light of the detailed disclosure provided herein.

For systemic administration, a therapeutically effective dose can be estimated initially from in vitro assays. For example, a dose can be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One skilled in the art could readily optimize administration to humans based on animal data.

Dosage amount and interval may be adjusted individually to provide plasma and/or tissue levels of the inhibitors which are sufficient to maintain therapeutic effect. Usual patient dosages for administration by injection range from about 0.1 to 5 mg/kg/day, preferably from about 0.5 to 1 mg/kg/day. Therapeutically effective serum levels may be achieved by administering multiple doses each day. Suitable dosage ranges for intranasal administration are generally about 0.01 pg/kg body weight to 1 mg/kg body weight. Effective doses may be extrapolated from dose-response curves derived from in vitro or animal model test systems.

In cases of local administration or selective uptake, the effective local concentration of the inhibitors may not be related to plasma concentration. One skilled in the art will be able to optimize therpeautically effective local dosages without undue experimentation.

The amount of inhibitor administered will, of course, be dependent on the subject being treated, on the subject's weight, the severity of the affliction, the manner of administration and the judgment of the prescribing physician.

The therapy may be repeated intermittently while the pain is detectable or even when they are not detectable. The therapy may be provided alone or in combination with other drugs.

H. Toxicity

Preferably, a therapeutically effective dose of the inhibitors for the Ras-MEK-ERK1/2 cascade, and/or PKA cascade, and/or the PKCε cascade described herein will provide therapeutic benefit without causing substantial toxicity.

Toxicity of the inhibitors described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. Inhibitors which exhibit high therapeutic indices are preferred. The data obtained from these cell culture assays and animal studies can be used in formulating a dosage range that is not toxic for use in human. The dosage of the inhibitors described herein lies preferably within a range of circulating concentrations that include the effective dose with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, In: The Pharmacological Basis of Therapeutics, Ch.1, p.1).

XI. Kits.

In certain embodiments, this nvention provides kits for practice of the methods of this invention. The kits can include a container containing one or more Ras-MEK-ERK 1/2 modulators. The modulator(s) can be provided in a pharmaceutically acceptable excipient and/or in a unit dosage formulation.

In certain embodiments, the kits additionally include instructional materials teaching the use of one or more Ras-MEK-ERK 1/2 modulators in the treatment of pain. While the instructional materials typically comprise written or printed materials they are not limited to such. Any medium capable of storing such instructions and communicating them to an end user is contemplated by this invention. Such media include, but are not limited to electronic storage media (e.g., magnetic dsiscs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

Nociceptor Sensitization by Extracellular Signal-Regulated Kinases

Inflammatory pain, characterized by a decrease in mechanical nociceptive threshold (hyperalgesia), arises through actions of inflammatory mediators, many of which sensitize primary afferent nociceptors via G-protein-coupled receptors. Two signaling pathways, one involving protein kinase A (PKA) and one involving the epsilon isozyme of protein kinase C (PKCε), have been implicated in primary afferent nociceptor sensitization. Here we describe a third, independent pathway that involves activation of extracellular signal-regulated kinases (ERKs) 1 and 2. Epinephrine, which induces hyperalgesia by direct action at β2-adrenergic receptors on primary afferent nociceptors, stimulated phosphorylation of ERK1/2 in cultured rat dorsal root ganglion cells. This was inhibited by a β2-adrenergic receptor blocker and by an inhibitor of mitogen and extracellular signal-regulated kinase kinase (MEK), which phosphorylates and activates ERK1/2. Inhibitors of Gi/o-proteins, Ras farnesyltransferases, and MEK decreased epinephrine-induced hyperalgesia. In a similar manner, phosphorylation of ERK1/2 was also decreased by these inhibitors. Local injection of dominant active MEK produced hyperalgesia that was unaffected by PKA or PKCε inhibitors. Conversely, hyperalgesia produced by agents that activate PKA or PKCε was unaffected by MEK inhibitors. We conclude that a Ras-MEK-ERK1/2 cascade acts independent of PKA or PKCε as a novel signaling pathway for the production of inflammatory pain. This pathway may present a target for a new class of analgesic agents.

Introduction

Current evidence indicates that at least two signaling pathways mediate hyperalgesia produced by inflammatory agents. The inflammatory mediators prostaglandin E₂ (PGE₂), serotonin, and adenosine produce hyperalgesia through activation of protein kinase A (PKA) (Gold M S, et al. (1996) Hyperalgesic agents increase a tetrodotoxin-resistant Na+current in nociceptors. Proc Natl Acad Sci USA 93:1108-1112; Gold M S, et al. (1998) Modulation of TTX-R I Na by PKC and PKA and their role in PGE2-induced sensitization of rat sensory neurons in vitro. J Neurosci 18:10345-10355; Khasar S G, et al. (1998a) A tetrodotoxin-resistant sodium current mediates inflammatory pain in the rat. Neurosci Lett 256:17-20; and, Khasar S G, et al. (1999a) A novel nociceptor signaling pathway revealed in protein kinase Cε mutant mice. Neuron 24:253-260.), and this process is facilitated by nitric oxide (Aley K. O., et al. (1998) Nitric oxide signaling in pain and nociceptor sensitization in the rat. J Neurosci 18:7008-7014; and, Chen X, & Levine J D (1999) NOS inhibitor antagonism of PGE2-induced mechanical sensitization of cutaneous C-fiber nociceptors in the rat. J Neurophysiol 81:963-966). On the other hand, epinephrine, acting through β2-adrenergic receptors on primary afferent nociceptors, produces mechanical hyperalgesia in part through PKA but also through the epsilon isozyme of protein kinase C (PKCε) (Khasar S G, et al., (1999a), supra). PKCε also contributes to bradykinin-induced sensitization of nociceptors to heat (Cesare P, et al. (1999) Specific involvement of PKC-epsilon in sensitization of the neuronal response to painful heat. Neuron 23:617-624).

PKA and PKCε mediate nociceptor sensitization by modulating the activity of a tetrodotoxin-resistant sodium current that is sensitized by direct-acting hyperalgesic agents (Gold et al., 1996, supra; Khasar et al., 1999a, supra; and, Khasar et al. (1999b) Epinephrine produces a beta-adrenergic receptor-mediated mechanical hyperalgesia and in vitro sensitization of rat nociceptors. J Neurophysiol 81:1104-1112). We originally thought that PKA and PKCε signaling pathways might converge at extracellular signal-regulated kinases 1 and 2 (ERK1/2), because ERK1/2 are modulated by PKA and PKCε (Hundle B, et al. (1995) Overexpression of,-protein kinase C enhances nerve growth factor-induced phosphorylation of mitogen-activated protein kinases and neurite outgrowth. J Biol Chem 270:30134-30140; Vossler M R, et al. (1997) cAMP activates MAP kinase and Elk-1 through a B-Raf- and Rap1-dependent pathway. Cell 89:73-82; and, Grewal S S, et al. (2000) Neuronal calcium activates a Rap1 and B-Raf signaling pathway via the cyclic adenosine monophosphate-dependent protein-kinase. J Biol Chem 275:3722-3728.). Moreover, β2-adrenergic receptors, like several other G-protein-coupled receptors, can activate ERKs (Daaka Y, et al. (1997) Switching of the coupling of the β2 adrenergic receptor to different G proteins by protein kinase A. Nature 390:88-91; Della Rocca G J, et al. (1997) Ras-dependent mitogen-activated protein kinase activation by G protein-coupled receptors. J Biol Chem 272:19125-19132; Wan Y, & Huang X-Y (1998) Analysis of the Gs/mitogen-activated protein kinase pathway in mutant S49 cells. J Biol Chem 273:14533-14537; Maudsley S, et al. (2000) The β2-adrenergic receptor mediates extracellular signal-regulated kinase activation via assembly of a multi-receptor complex with the epidermal growth factor receptor. J Biol Chem 275:9572-9580; and, Schmitt J M, & Stork P J S (2000) β2-adrenergic receptor activates extracellular signal-regulated kinases (ERKs) via the small G protein Rap1 and the serine/threonine kinase B-Raf. J Biol Chem 275:25342-25350). ERKs are mitogen-activated protein (MAP) kinases that mediate several cellular responses to mitogenic and differentiation signals (Lewis T S, et al. (1998) Signal transduction through MAP kinase cascades. Adv Cancer Res 74:49-139). They are activated by diverse extracellular stimuli, including several hormones and growth factors that activate G-protein-coupled receptors or receptor tyrosine kinases, leading to stimulation of Raf kinases, which phosphorylate and activate mitogen and extracellular signal-regulated kinase kinase (MEK). Activated MEK in turn phosphorylates and activates ERK1/2. PKA and cAMP can promote ERK activation via a Rap1-dependent pathway in neural cells, such as PC12 cells, that use B-Raf as the major Raf isoform (Ohtsuka T, et al., (1996) Activation of brain B-Rafprotein kinase by Rap1B small GTP-binding protein. J Biol Chem 271:1258-1261; Vossler et al., (1997), supra; Kawasaki H, et al., (1998) A family of cAMP-binding proteins that directly activate Rap1. Science 282:2275-2279; York R D, et al., (1998) Rap1 mediates sustained MAP kinase activation induced by nerve growth factor. Nature 392:622-626; and, Grewal et al., (2000), supra). In PC12 cells, PKCε promotes ERK phosphorylation and activation by nerve growth factor (NGF) or epidermal growth factor (EGF) through an unknown mechanism (Hundle et al., (1995), supra; Hundle B, et al., (1997) An inhibitory fragment derived from protein kinase C ε prevents enhancement of nerve growth factor responses by ethanol and phorbol esters. J Biol Chem 272:15028-15035; and, Brodie C, et al., (1999) Protein kinase C-epsilon plays a role in neurite outgrowth in response to epidermal growth factor and nerve growth factor in PC12 cells. Cell Growth Differ 10:183-191). Thus, activation of ERKs in nociceptors could provide an important mechanism for convergence of PKA and PKCε signaling pathways.

In this paper, we examined whether ERK activation is involved in pain signaling by examining epinephrine-treated rat dorsal root ganglion (DRG) neurons in culture and epinephrine-induced mechanical hyperalgesia in rats. We report that epinephrine activates ERKs in cultured DRG neurons and that a heterotrimeric Gi -or Go-protein, Ras, and MEK contribute to epinephrine-induced hyperalgesia, independent of PKCε or PKA.

Material and Methods

Materials

Epinephrine, the selective β2-adrenergic receptor antagonist ICI 118,551, PGE₂, pertussis toxin, epinephrine, and the isoprenylation inhibitor perillic acid were purchased from Sigma (St. Louis, Mo.). The general PKC inhibitor bisindoylmaleimidel (BIM), the PKA inhibitor H89, the MEK inhibitors U0126 and PD98059, mouse 2.5 S NGF, and the farensyltransferase inhibitor FTase I were from Calbiochem (La Jolla, Calif.). The Walsh inhibitor peptide (WI PTI DE) of PKA was purchased from Peninsula Laboratories (Belmont, Calif.). Anti-phospho-p42/44 MAP kinase (Thr202/Tyr204) antibody against the MEK-phosphorylated forms of ERK1/2 and anti-ERK1/2 antibody were purchased from New England Biolabs (Beverly, Mass.) or, where indicated, from Upstate Biotechnology (Lake Placid, N.Y.). Dominant active and kinase inactive recombinant MEK1 were purchased from Upstate Biotechnology. A specific activator of PKCε, ΨεRACK (receptor for activated C kinase), was a gift from D. Mochly-Rosen (Stanford University, Stanford, Calif.). A specific inhibitor of PKCε, εV1-2, was synthesized by SynPep (Danville, Calif.).

Cell Culture

Dorsal root ganglia were collected from male adult Sprague Dawley rats (200 gm) obtained from Simonsen (Gilroy, Calif.) or from PKCε null and wild-type C57BL/6J×129 SvJae mice of the F2 generation (Khasar et al., (1999a), supra). The cells were dissociated by treating ganglia with 0.125% collagenase P for 2 hr, followed by a trypsin solution 0.025% trypsin and 0.025% EDTA in HBSS) for 15 min. Trypsin was inactivated by adding 100 μg/ml soybean trypsin inhibitor and 2.5 mg/ml MgSO₄. The cells were centrifuged at 300×g for 5 min and resuspended culture media containing minimal essential medium (MEM) supplemented with 10% heat-inactivated fetal calf serum, 1×MEM vitamins, and 1000 U/ml each of penicillin and streptomycin. The culture was enriched for neurons by preplating on 100 mm culture dishes pretreated with 0.1 mg/ml poly-DL-ornithine in 15 mM sodium borate buffer. After culture for 15-20 hr, the loosely attached neuronal cells were collected and plated for 3 hr on six-well plates coated with 0.1 mg/ml poly-DL-ornithine and 1 mg/ml laminin.

Western Analysis.

After drug treatment, cells from neuron-enriched DRG cultures were collected and centrifuged at 300×g for 5 min at 4° C. The pellets were resuspended in lysis buffer [50 mM Tris HCl, pH 7.4, 1% (v/v) NP-40, 0.25% sodium deoxycholate, 150 mM NaCl, 1 mM EGTA, and 10 mM EDTA] and protease inhibitors (leupeptin and aprotinin at 40 μg/ml each, 25 μg/ml soybean trypsin inhibitor, and 1 mM PMSF) and phosphatase inhibitors (25 mM NaF, 1 mM Na3 VO₄, 40 mM β glycero-phosphate, and 1 mM Na pyrophosphate). Proteins in 200 μg samples of cell lysates were separated by SDS-PAGE using 12% polyacrylamide gels. The proteins were electroblotted onto Hybond C nitrocellulose membranes, which were incubated in a blocking solution containing 5% nonfat dry milk dissolved in PBS-T (137 mM NaCl, 2.7 mM KCl, 1.47 mM KH2 PO₄, 8 mM NaHPO₄, 0.5 mM MgCl₂, and 0.9 mM CaCl₂, pH 7.2, and 0.1% Tween 20). Blots were incubated with anti-phospho-p42/44 MAP kinase antibody (diluted 1:500 in blocking buffer) overnight at 4° C. Blots were rinsed three times in PBS-T and incubated in blocking buffer containing HRP-conjugated goat anti-rabbit IgG (diluted 1:1000; Boehringer Mannheim, Indianapolis, Ind.) for 1 hr at 27° C. Immunoreactive bands were visualized by enhanced chemiluminescence (Amersham Pharmacia Biotech, Piscataway, N.J.). The membranes were then stripped of antibodies by incubation in 200 mM NaOH for 20 min at 27° C. After three washes in PBS-T, blots were incubated with anti-ERK1/2 antibody (136 ng/ml) for 1 hr at 27° C. After three washes in PBS-T, blots were incubated with HRP-conjugated goat anti-rabbit IgG (diluted 1:1000; Boehringer Mannheim) for 1 hr at 27° C., and immunoreactive bands were visualized by enhanced chemiluminescence and autoradiography. Immunoreactive bands on autoradiograms were analyzed by scanning densitometry using a flatbed scanner and NIH Image version 1.62 (W. Ras-band, National Institutes of Health, Bethesda, Md.). Data were normalized by dividing values obtained for phospho-ERK1 and phospho-ERK2 immunoreactivity by the value obtained for total ERK1 immunoreactivity for each sample.

Immunofluorescence.

Adult male Sprague Dawley rats were anesthetized with pentobarbital and transcardially perf used with PBS, followed by 4% paraformaldehyde (in PBS). DRGs were removed, post-fixed in 4% paraformaldehyde for 4 hr, treated with 30% sucrose (in PBS) for 24 hr, and then embedded in Tissue-Tek OCT. Cryosections (8 μm) were cut and stored at −20° C. Mounted DRG sections were allowed to thaw to room temperature. Sections were then incubated for 1 hr in blocking solution (PBS containing 5% normal donkey serum and 0.1% Triton X-100), overnight with anti-ERK1/2 (0.82 μg/ml; Upstate Biotechnology) and 1 hr with FITC-conjugated donkey anti-rabbit (7.5 μg/ml; Jackson ImmunoResearch, West Grove, Pa.). Both primary and secondary anti-bodies were diluted in 1.5% normal donkey serum, in PBS.

Animal Housing

For behavioral studies, male Sprague Dawley rats (200-250 gm; Bantin-Kingman, Fremont, Calif.) were individually housed and maintained under a 12 hr light/dark cycle. The experimental rats were fed standard lab chow ad libitum. All experimental procedures were approved by the Institutional Animal Care and Use Committee of the University of California, San Francisco.

Mechanical Nociceptive Threshold.

The nociceptive flexion reflex (Randall-Selitto paw-withdrawal test) was quantified with a Basile Analge-symeter (Stoelting, Chicago, Ill.), which applies a linearly increasing mechanical force to the dorsum of the rat's hindpaw. The mechanical nociceptive threshold was defined as the force in grams at which the rat withdrew its paw. On the day of the test, animals were brought to the laboratory and allowed to remain in the cage for 10-15 min. They were allowed to crawl into individual cylindrical Perspex blocks and were lightly restrained there by closing both ends of the cylinder. The hind-paws of the rats were freed out of the cylinder through triangular slits on either side of the Perspex block, which allows easy access to the hindpaws during the test (Aley K O, Levine J D (1999) Role of protein kinase A in the maintenance of inflammatory pain. J Neurosci 19:2181-2186). The rats were allowed to acclimatize to the restrainer for 5-10 min, after which the hindpaws were exposed to the test stimulus. Three readings were taken at 5 min intervals, and their mean was considered the baseline threshold. After each drug administration, mechanical paw-withdrawal thresholds were determined again as the mean of three readings taken 20, 25, and 30 min after injection. The result was expressed as the percentage decrease in nociceptive threshold [(paw-withdrawal threshold after the drug-basal paw withdrawal threshold)/basal withdrawal threshold×100].

Drugs for In Vivo Studies.

Stock solutions (1 μg/μl) of BIM (in 10% dimethylsulfoxide) and εV1-2 and WI PTI DE (in 0.9% saline) were stored at 20° C. Inhibitors were diluted with distilled water before intra-dermal injections into the paw using a 10 μl microsyringe (Hamilton, Reno, Nev.). Injections of peptides (1 μg/2.5 μl) were always preceded by injection of distilled water (2.5 μl) to produce hypo-osmotic shock. This was done to increase cell membrane permeability to these agents (Tsapis A, & Kepes A (1977) Transient breakdown of the permeability barrier of the membrane of Escherichia coli upon hypoosmotic shock. Biochim Biophys Acta 469:1-12; West L K, & Huang L (1980) Transient permeabilization induced osmotically in membrane vesicles from Torpedo electroplax: a mild procedure for trapping small molecules. Biochemistry 19:4418-4423; Taiwo Y O, & Levine J D (1989) Contribution of guanine nucleotide regulatory proteins to prostaglandin hyperalgesia in the rat. Brain Res 492:400-403; Khasar S G, et al., (1995) Mu-opioid agonist enhancement of prostaglandin-induced hyperalgesia in the rat: a G-protein beta gamma subunit-mediated effect? Neuroscience 67:189-195; and, Widdicombe J H, et al., (1996) Transient permeabilization of airway epithelium by mucosal water. J Appl Physiol 81:491-499). The dose of each protein kinase inhibitor was separated from the distilled water by an air bubble (<1 μl) so that the distilled water was injected into the paw first. Paw-withdrawal thresholds were measured again 10, 15, and 20 min after injecting the test agent. The mean of the paw-withdrawal thresholds obtained at these three times was then taken as the mechanical nociceptive threshold at the dose of the test agent used. The effect of each dose a test agent was calculated as the percentage change from baseline.

Statistical Analysis.

The data are presented as mean ±SE values and were compared using the one way ANOVA, followed by Newman-Keuls, Tukey's, or Dunnett's post hoc tests, as noted. Differences between means were considered significant at p<0.05.

Results

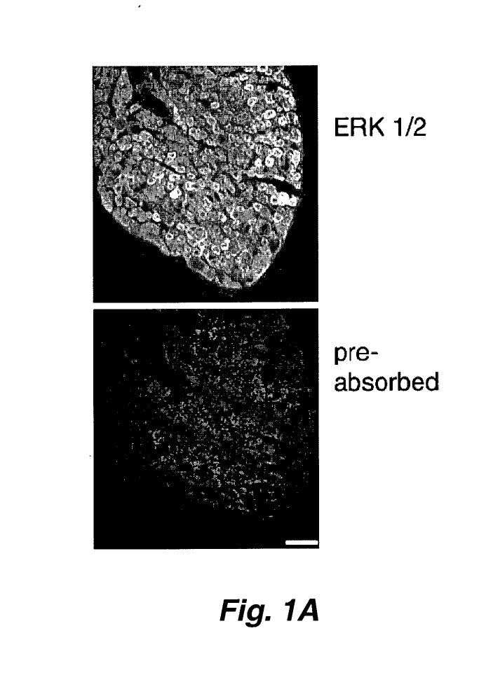

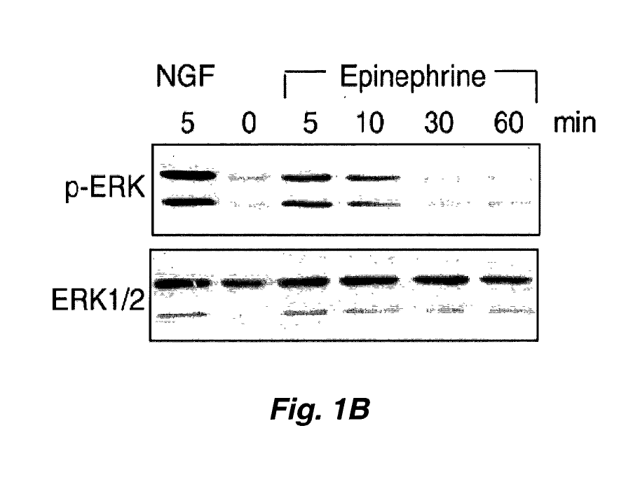

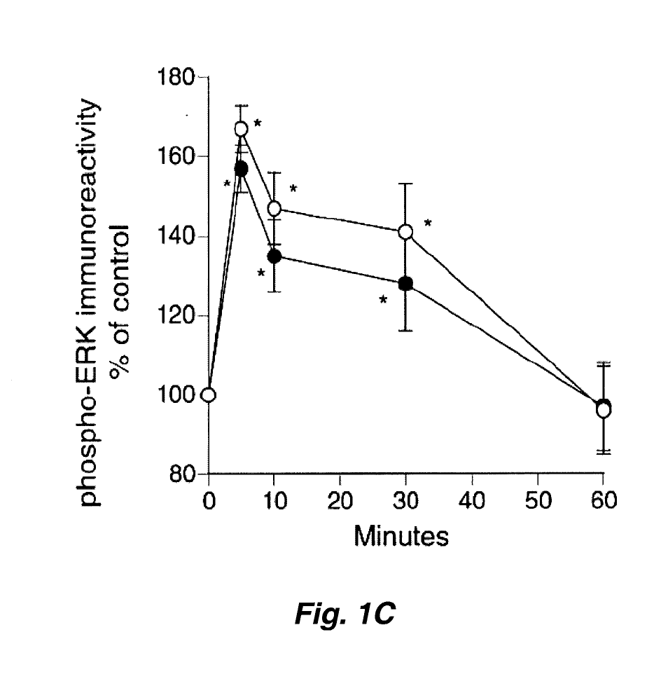

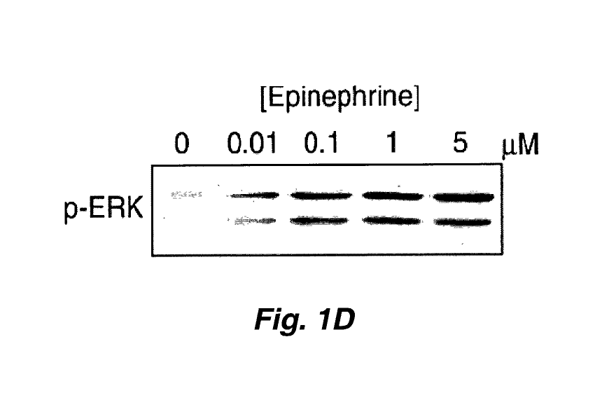

Because of the recent evidence linking β 2-adrenergic receptor stimulation to activation of ERKs in non-neuronal cells (Daaka et al., (1997), supra; Della Rocca et al., (1997), supra; Wan and Huang, (1998), supra; Maudsley et al., (2000), supra; and, Schmitt and Stork, (2000), supra), we examined whether ERK1/2 are present in rat DRG neurons and are activated by epinephrine. Immunofluorescence staining of isolated DRG demonstrated ERK1/2 immunoreactivity in cell bodies of DRG neurons (FIG. 1A). To measure responses to epinephrine, we next examined DRG neurons in culture. In unstimulated neurons, there was a basal level of phospho-ERK immunoreactivity (FIGS. 1B, C). This was increased by ˜1.7-fold after incuba-tion with 1 μM epinephrine. Epinephrine-evoked ERK phosphorylation was greatest after 5 min and gradually returned to basal levels after 60 min. The effect of epinephrine was dose-dependent and appeared to be maximal at a concentration of 1 μM (FIG. 1D). The maximal response to epinephrine was ˜30-50% of the response observed after incubation with a maximally effective concentration of NGF (50 ng/ml) for 5 min (FIG. 1B).

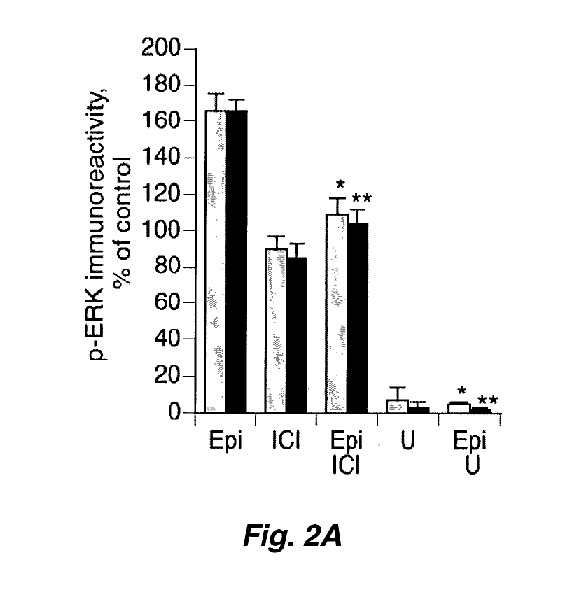

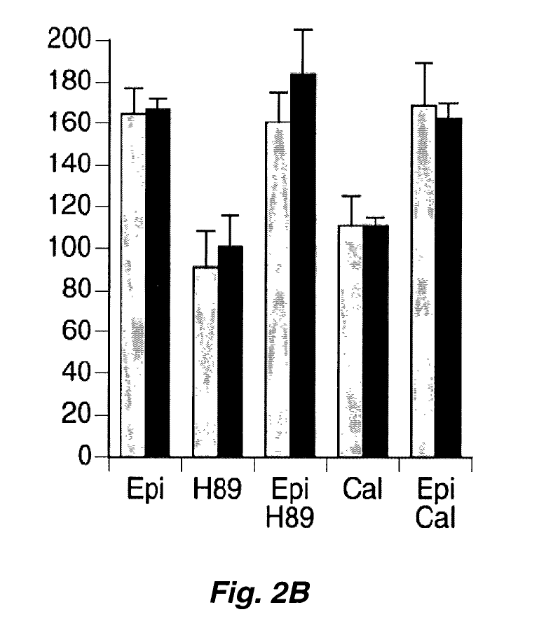

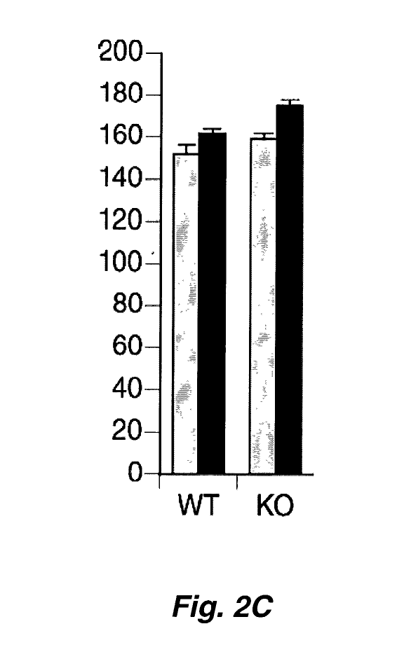

Epinephrine-induced stimulation of ERK phosphorylation was mediated by β 2-adrenergic receptors and MEK because it was inhibited by ICI 118,551 (Samama P, et al., (1994) Negative antagonists promote an inactive conformation of the beta 2-adrenergic receptor. Mol Pharmacol 45:390-394) and by the selective MEK inhibitor U0126 (Favata et al., (1998) Identification of a novel inhibitor of mitogen-activated protein kinase kinase. J Biol Chem 273:18623-18632) (FIG. 2A). ICI 118,551, which is an inverse agonist, did not inhibit basal ERK phosphorylation, suggesting that basal activity of the β 2 receptor does not contribute significantly to the low level of phosphorylated ERK observed in unstimulated cultures.

Our previous studies indicated that signaling pathways involving PKA and PKCε are important for primary afferent nociceptor sensitization induced by activation of β 2-adrenergic receptors (Khasar et al., (1999a), supra; and, Khasar et al., (1999b), supra). Therefore, we examined whether these kinases lie in a signaling pathway that includes MEK and ERK in DRG neurons. We found that treatment with H89, which inhibits PKA (Chijiwa et al., (1990) Inhibition of forskolin-induced neurite outgrowth and protein phosphorylation by a newly synthesized selective inhibitor of cyclic AMP-dependent protein kinase, N-[2-(p-bromocinnamylamino)ethyl]-5-isoquinolinesulfonamide (H-89), of PC12D pheochromocytoma cells. J Biol Chem 265:5267-5272), or calphostin C, which inhibits several PKC isozymes including PKCε (Mayne and Murray, (1998) Evidence that protein kinase C epsilon mediates phorbol ester inhibition of calphostin C- and tumor necrosis factor-alpha-induced apoptosis in U937 histiocytic lymphoma cells. J Biol Chem 273:24115-24121), did not reduce epinephrine-stimulated ERK phosphorylation (FIG. 2B). Moreover, treatment with epinephrine evoked similar levels of ERK phosphorylation in mouse DRG cultures obtained from wild-type and PKCε null mice (FIG. 2C). These findings indicate that epinephrine stimulates ERK phosphorylation in DRG neurons through a signaling pathway that does not involve PKA or PKCε.

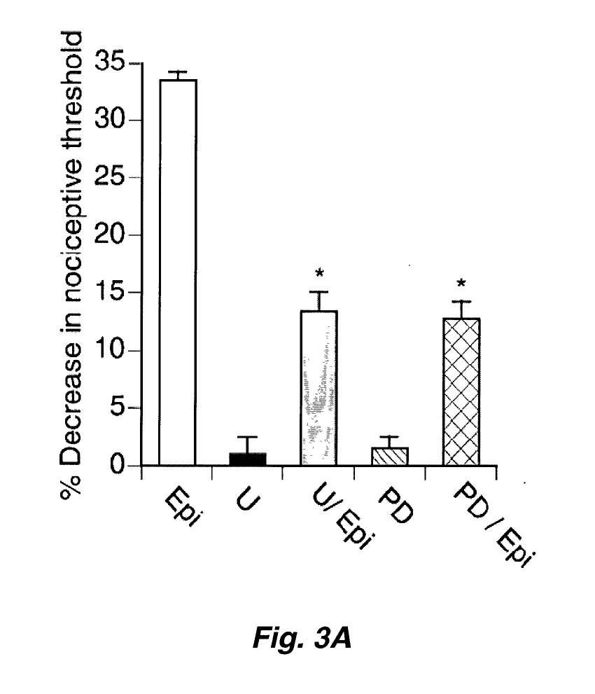

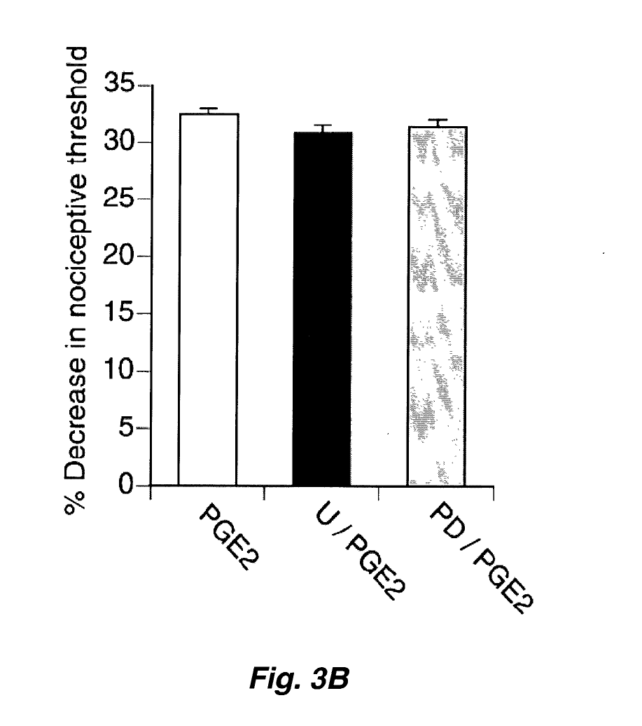

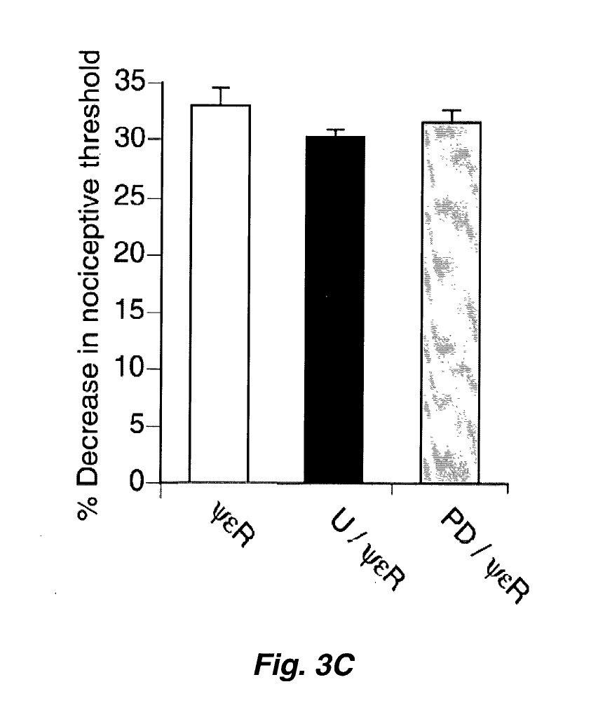

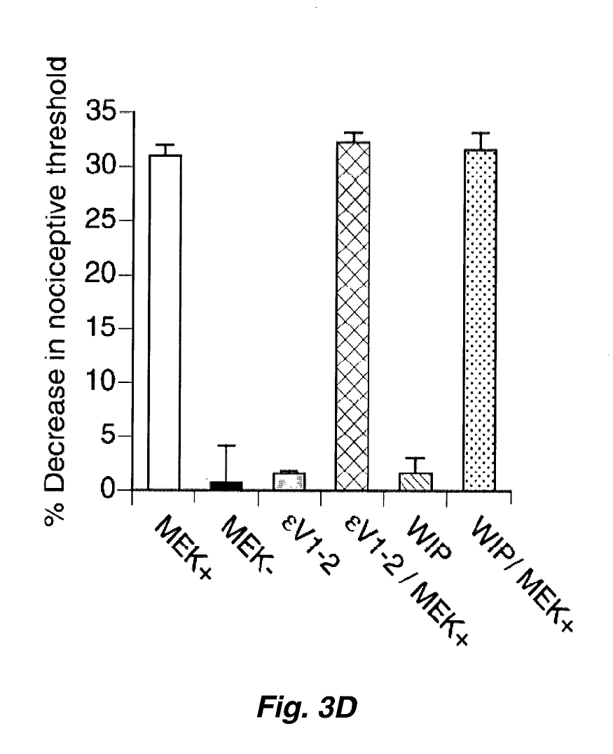

We next evaluated whether MEK contributes to epinephrine-induced hyperalgesia and whether this is independent of PKA or PKCε. Intradermal injection of epinephrine decreased mechanical nociceptive thresholds by ˜35%, and this effect was inhibited by the MEK inhibitors U0126 and PD98059 (Favata et al., (1998), supra) (FIG. 3A). However, U0126 and PD98059 had no effect on hyperalgesia induced by PGE₂, which requires PKA activation for its pronociceptive effect (Aley and Levine, (1999), supra; and, Chen and Levine, (1999), supra), or by ψεRACK, a specific activator of PKCε (Dorn et al., (1999) Sustained in vivo cardiac protection by a rationally designed peptide that causes epsilon protein kinase C translocation. Proc Natl Acad Sci USA 96:12798-12803; and, Aley et al., (2000) Chronic hypersensitivity for inflammatory nociceptor sensitization mediated by the epsilon isozyme of protein kinase C. J Neurosci 20:4680-4685). Treatment with a dominant active MEK mutant was sufficient to induce hyperalgesia (FIG. 2D). This effect required the kinase activity of MEK because a kinase-dead MEK mutant was ineffective. Treatment with the specific PKCε inhibitor εV1-2 (Johnson et al., (1996) A protein kinase C translocation inhibitor as an isozyme-selective antagonist of cardiac f unction. J Biol Chem 271:24962-24966; and, Khasar et al., (1999a), supra) or with the PKA inhibitor WIPTIDE did not reduce hyperalgesia induced by active MEK (FIG. 2D). These findings indicate that MEK mediates epinephrine-induced mechanical hyperalgesia through a signaling pathway that is independent of PKA or PKCε.

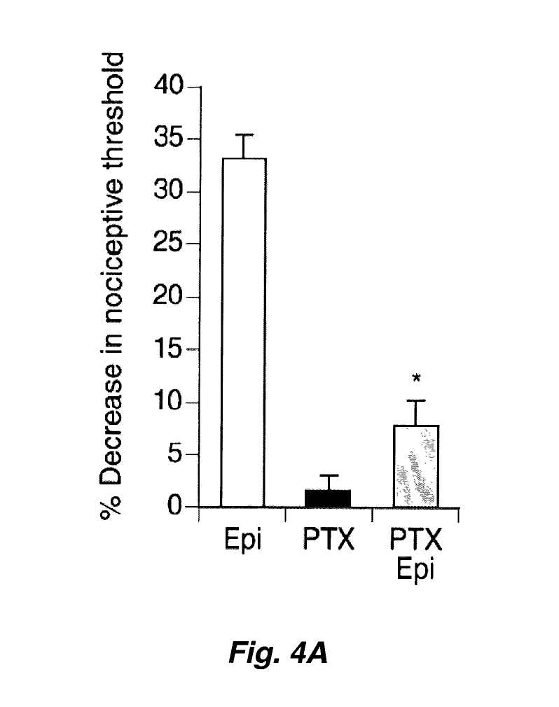

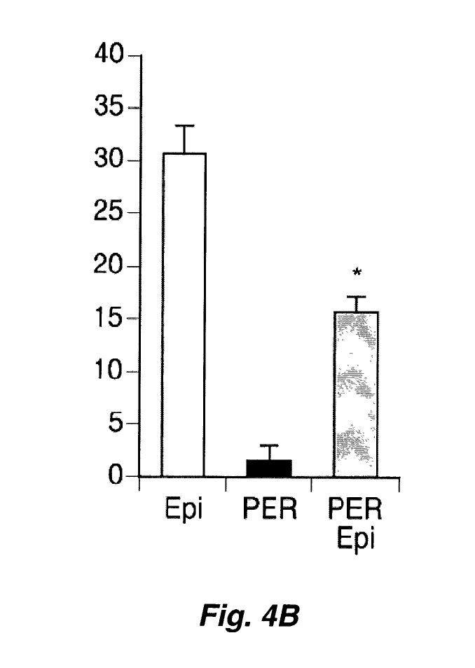

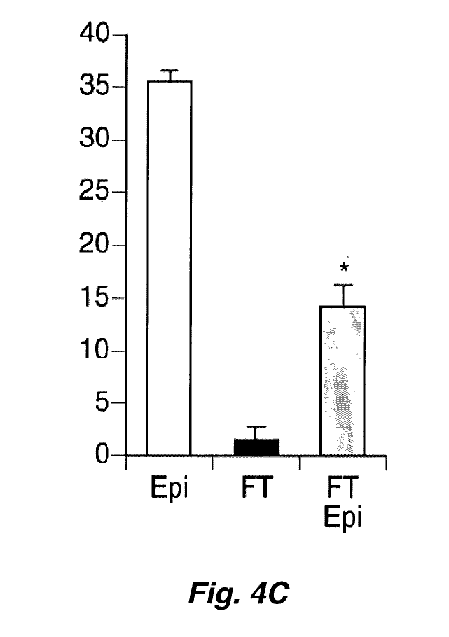

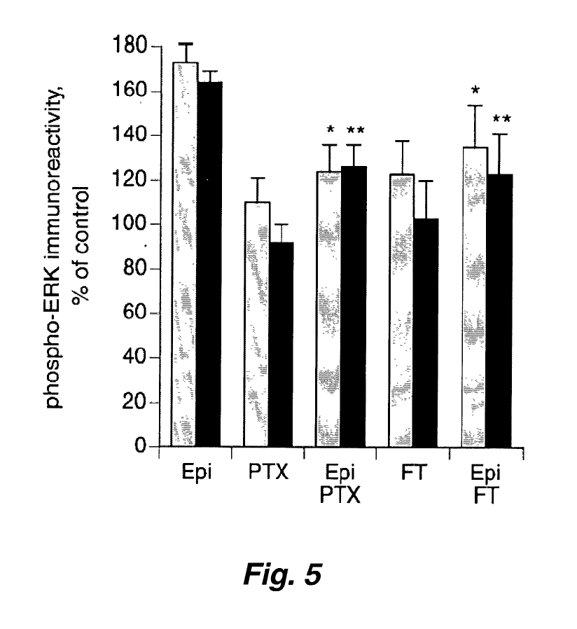

In human embryonic kidney 293 (HEK293) cells transfected to overexpress β 2-adrenergic receptors, β 2 agonists activate ERKs through a signaling cascade that involves a Gi/o-protein and Ras (Daaka et al., (1997), supra; and, Della Rocca et al., (1997), supra), whereas stimulation of endogenous β 2 receptors activates ERKs through a pathway involving Gs and Rap-1 (Schmitt and Stork, (2000), supra). To examine pathways involved in β 2-mediated activation of ERKs in DRG neurons, we treated rat DRG cultures with pertussis toxin to inactivate Gi/o and with the isoprenylation inhibitor perillic acid (Hardcastle et al., (1999) Inhibition of protein prenylation by metabolites of limonene. Biochem Pharmacol 57:801-809). Both of these agents reduced epinephrine-mediated mechanical hyperalgesia (FIG. 4A, B). Small GTPases are generally modified post-translationally by the addition of the isoprenaloids famesyl or geranylgeranyl to a cysteine residue near the C terminus (Zhang and Casey, (1996) Protein prenylation: molecular mechanisms and functional consequences. Annu Rev Biochem 65:241-269). Ras proteins are preferentially farresylated, and inhibitors of famesyltransferase block the transforming ability of H-Ras (Kohl et al., (1994) Protein farnesyltransferase inhibitors block the growth of ras-dependent tumors in nude mice. Proc Natl Acad Sci USA 91:9141-9145). In contrast, Rap1A and B, which have a leucine residue at their C termini, are preferentially geranylgeranylated. Treatment with FTase I, which inhibits Ras farnesylation, attenuated epinephrine-mediated mechanical hyperalgesia (FIG. 4C). Similarly, pertussis toxin and FTase I prevented epinephrine-induced phosphorylation of ERK1/2 (FIG. 5). These studies suggest that a Gi/o-Ras-ERK1/2 pathway contributes to epinephrine-induced hyperalgesia.

Discussion

Second-messenger signaling pathways involving PKA and PKCε have been implicated previously in nociceptor sensitization (Khasar et al., (1999a), supra; and, Khasar et al., (1999b), supra). This report provides the first demonstration of a role for ERK signaling in this process. Using kinase-selective inhibitors, we found that epinephrine-induced phosphorylation of ERK1/2 is independent of PKA and PKCε. In vivo, activated MEK was sufficient to cause a hyperalgesia that does not require PKA or PKCε. Conversely, PKCε- and PKA (PGE₂)-mediated hyperalgesia was independent of MEK activity. Therefore, ERKs, PKA, and PKCε appear to define three independent signaling pathways that mediate nociceptor sensitization by inflammatory mediators.

β 2-adrenergic receptor activation stimulates ERK phosphorylation in HEK293 cells (Daaka et al., (1997), supra; Della Rocca et al., (1997), supra; and, Schmitt and Stork, (2000), supra), COS-7 cells (Maudsley et al., (2000), supra), S49 lymphoma cells (Wan and Huang, (1998), supra), and cardiac myocytes (Zou et al., (1999) Both Gs and Gi proteins are critically involved in isoproterenol-induced cardiomyocyte hypertrophy. J Biol Chem 274: 9760-9770). Some of the most detailed studies have been performed with HEK293 cells in which endogenous β 2 receptors activate ERKs through a pathway involving Gs, PKA, Rap1, and B-Raf (Schmitt and Stork, (2000), supra). Without being bound by a particular theory, it is unlikely that Rap1 plays a role in β 2 receptor-mediated nociceptor sensitization because treatment with the PKA inhibitor H89 did not block epinephrine-induced ERK phosphorylation or mechanical hyperalgesia. In HEK293 cells transfected to overexpress β 2 receptors, epinephrine stimulates a different pathway resulting in activation of Gi/o and Src, and transactivation of EGF receptors leading to stimulation of Ras, MEK, and ERKs (Daaka et al., (1997), supra; Della Rocca et al., (1997), supra; and, Maudsley et al., (2000), supra). We found evidence to support involvement of Gi/o and Ras in β 2 receptor-mediated ERK activation in DRG neurons and hyperalgesia.

In addition to regulating gene expression, cell proliferation, differentiation, development, and apoptosis (Lewis et al., (1998), supra), ERKs have been implicated in neural plasticity associated with learning and memory (Bailey et al., (1997) Mutation in the phosphorylation sites of MAP kinase blocks learning-related internalization of apCAM in Aplysia sensory neurons. Neuron 18:913-924; and, Martin et al., (1997) MAP kinase translocates into the nucleus of the presynaptic cell and is required for long-term facilitation in Aplysia. Neuron 18:899-912). Here we demonstrate a role for ERK signaling in another form of neural plasticity, namely sensitization of nociceptors. The downstream effectors of ERKs that mediate nociceptor sensitization are not known. Because modulation of voltage-sensitive potassium channels may contribute to nociceptor sensitization (Evans et al., (1999) The cAMP transduction cas-cade mediates the PGE₂-induced inhibition of potassium currents in rat sensory neurones. J Physiol (Lond) 516:163-178), recent findings that the A-type K⁺ channel Kv4.2 is a substrate for ERK1/2 (Adams et al., (2000) The A-type potassium channel Kv4.2 is a substrate for the mitogen-activated protein kinase ERK. J Neurochem 75:2277-2287) may provide such a downstream target. Production of arachidonic acid metabolites by cytoplasmic phospholipase A2, another substrate of ERK1/2 (Lin et al., (1993) cPLA2 is phosphorylated and activated by MAP kinase. Cell 72:269-278), has been implicated in signaling pathways involved in nociceptor function (Hwang et al., (2000) Direct activation of capsaicin receptors by products of lipoxygenases: endogenous capsaicin-like substances. Proc Natl Acad Sci USA 97:6155-6160; and, Piomelli, (2001) The ligand that came from within. Trends Pharmacol Sci 22:17-19). Thus, at least two known ERK1/2 substrates are potential mediators of nociceptor sensitization induced by ERK signaling.

Activation of ERKs by β-adrenergic receptor stimulation may contribute to inflammatory pain, because increased levels of epinephrine are found at sites of inflammation (Mikhailov and Rusanova, (1993) The interrelationship of the catecholamine and protein content of the tissue of the submandibular salivary glands and the mucosa during the secretory cycle in chronic inflammation of the oral soft tissues. Biull Eksp Biol Med 116:472-474) and β-adrenergic receptor antagonists reduce inflammatory hyperalgesia (Cunha et al., (1991) Interleukin-8 as a mediator of sympathetic pain. Br J Pharmacol 104:765-767). Catecholamines released from sympathetic nerve terminals and from the adrenal medulla also appear to contribute to sympathetically maintained pain and stress-aggravated pain (Choi and Rowbotham, (1997) Effect of adrenergic receptor activation on post-herpetic neuralgia pain and sensory disturbances. Pain 69:55-63; and, Khasar et al., (1998b) Vagotomy-induced enhancement of mechanical hyperalgesia in the rat is sympathoadrenal-mediated. J Neurosci 18:3043-3049). When injected intradermally, epinephrine lowers the nociceptive threshold with an ED₅₀ of ˜20 ng (Khasar et al., (1999b), supra), which is similar to the ED₅₀ for bradykinin and PGE₂ (Khasar et al., (1993) Comparison of intradermal and subcutaneous hyperalgesic effects of inflammatory mediators in the rat. Neurosci Lett 153:215-218). In addition to catecholamines, NGF (Safieh-Garabedian et al., (1997) Involvement of interleukin-1 beta, nerve growth factor and prostaglandin E₂ in endotoxin induced localized inflammatory hyperalgesia. Br J Pharmacol 121:1619-1626) and bradykinin (Levine and Taiwo, (1994) Inflammatory pain. In: Textbook of pain (Wall P D, Melzack R, eds), pp 45-56. Edinburgh: Churchill Livingstone) are two other hyperalgesic mediators released during inflammation and tissue damage that can activate ERK1/2 (Clark and Murray, (1995) Evidence that the bradykinin-induced activation of phospholipase D and of the mitogen-activated protein kinase cascade involve different protein kinase C isoforms. J Biol Chem 270:7097-7103; and, Hundle et al., (1995), supra). Without being bound to a particular theory, this suggests that ERK signaling plays an important role in pain states evoked by several different mediators and pathological conditions. ERK pathways have several components, affording an opportunity for antagonism at many levels. Therefore, inhibition of ERK signaling in nociceptors may provide a fruitful strategy for the discovery of novel analgesics.

### Example 2

Sex Hormones Regulate the Contribution of PKCε and PKA Signalling in Inflammatory Pain in the Rat

We have evaluated the contribution of differences in second messenger signalling to sex differences in inflammatory pain and its control by sex hormones. In normal male but not female rats, epinephrine-induced mechanical hyperalgesia was antagonized by inhibitors of protein kinase C PKCε (PKCε), protein kinase A (PKA) and nitric oxide synthetase (NOS). Similarly, in PKCε knockout mice, a contribution of PKCε to epinephrine-dependent mechanical hyperalgesia occurred in males only. In contrast, hyperalgesia induced by prostaglandin E₂, in both females and males, was dependent on PKA and NO. In both sexes, inhibitors of mitogenactivated protein kinase/extracellular-signal related kinase kinase (MEK) inhibited epinephrine hyperalgesia. In gonadectomized females, the second messenger contributions to epinephrine hyperalgesia demonstrated the pattern seen in males. Administration of oestrogen to gonadectomized females fully reconstituted the phenotype of the normal female. These data demonstrate gender differences in PKCε, PKA and NO signalling in epinephrine-induced hyperalgesia which are oestrogen dependent and appear to be exerted at the level of the ●-adrenergic receptor or the G-protein to which it is coupled.

Introduction

Gender and sex hormone-related differences in pain (Romero & Bodnar, (1986) Gender differences in two forms of coldwater swim analgesia. Physiol. Behav., 37:893-897; Fillingim & Maixner, (1995) Gender differences in the responses to noxious stimuli. Pain Forum, 4:209-221; and, Unruh, (1996) Gender variations in clinical pain experience. Pain, 65:123-167) and nociception (Pare, (1969) Age, sex, and strain differences in the aversive threshold to grid shock in the rat. J. Comp Physiol. Psychol, 69:214-218; Kepler et al., (1989) Roles of gender, gonadectomy and estrous phase in the analgesic effects of intracerebroventricular morphine in rats. Pharmacol. Biochem. Behav., 34:119-127; Aloisi et al., (1994) Sex differences in the behavioural response to persistent pain in rats. Neurosci. Lett., 179:79-82; and, Coyle et al., (1995) Female rats are more susceptible to the development of neuropathic pain using the partial sciatic nerve ligation (PSNL) model. Neurosci. Lett., 186:135-138) have been described, and an important role for sex steroids (Beatty & Beatty, (1970) Hormonal determinants of sex differences avoidance behavior and reactivity to electric shock in the rat. J. Comp. Physiol. Psychol, 73:446-455; Marks et al., (1972) Reactivity to aversive stimuli as a function of alterations in body weight in nornal and gonadectomizedfemale rats. Physiol. Behav, 9:539-544; Romero et al., (1988) Genderspecific and gonadectomy-specific effects upon swim analgesia: role of steroid replacement therapy. Physiol. Behav, 44:257-265; Baamonde et al., (1989) Sex-related differences in the effects of morphine and stress on visceral pain. Neuropharmnacology, 28:967-970; Candido et al., (1992) Effect of adrenal and sex hormones on opioid analgesia and opioid receptor regulation. Pharmacol. Biochem. Behav, 42:685-692; and, Dawson-Basoa & Gintzler, (1993) 17-Beta-estradiol and progesterone modulate an intrinsic opioid analgesic system. Brain Res., 601:241-245) has been suggested. Whilst most of the literature in this area has addressed the modulatory role of sex steroids on CNS mechanisms of nociception, actions at the level of peripheral nociceptive mechanisms are also probable since both oestrogen and androgen receptors are present on small-diameter dorsal root ganglion (DRG) neurons (Sohrabji et al., (1994) Estrogen differentially regulates estrogen and nerve growth factor receptor mRNAs in adult sensory neurons. J. Neurosci., 14:459-471; Papka et al., (1997) Localization of estrogen receptor protein and estrogen receptor messenger RNA in peripheral autonomic and sensory neurons. Neuroscience, 79:1153-1163; and, Keast & Gleeson, (1998) Androgen receptor immunoreactivity is present in primary sensory neurons of male rats. Neuroreport, 9:4137-4140). Moreover, in DRG neurons oestrogen regulates the expression of mRNAs encoding trkA and p75 receptors (Sohrabji et al., (1994), supra) through which NGF signals (Kaplan et al., (1991) The trk proto-oncogene product: a signal transducing receptor for nerve growth factor. Science, 252:554-558) and presumably acts to produce its pronociceptive effects (Woolf, (1996) Phenotypic modification of primary sensory neurons: the role of nerve growth factor in the production of persistent pain. Phil. Trans. R. Soc. Lond. B Biol. Sci., 351:441-448; and, Okuse et al., (1997) Regulation of Expression of the Sensory Neuron-Specific Sodium Channel SNS in Inflammatory and Neuropathic Pain. Mol. Cell Neurosci., 10:196-207). Sex hormones also affect expression of protein kinase C (PKC) (including the e isoform, PKCε), protein kinase A (PKA) (Ansonoff & Etgen, (1998) Estradiol elevates protein kinase C catalytic activity in the preoptic area of female rats. Endocrinology, 139:3050-3056; Lavie et al., (1998) Tamoxifen induces selective membrane association of protein kinase C epsilon in MCF-7 human breast cancer cells. Int. J. Cancer, 77:928-932; Kelly et al., (1999) Rapid effects of estrogen to modulate G protein-coupled receptors via activation of protein kinase A and protein kinase C pathways. Steroids, 64:64-75; and, Han et al., (2000) Estradiol-17beta-BSA stimulates Ca (2+) uptake through nongenomic pathways in primary rabbit kidney proximal tubule cells: involvement of cAMP and PKC. J. Cell Physiol., 183:37-44) and nitric oxide synthetase activity, all of which are implicated in peripheral nociceptive mechanisms (Khasar et al., (1995) Is there more than one prostaglandin E receptor subtype mediating hyperalgesia in the rat hindpaw? Neuroscience, 64:1161-1165; Aley et al., (1998) Nitric oxide signaling in pain and nociceptor sensitization in the rat. J. Neurosci., 18:7008-7014; Aley & Levine, (1999) Role of protein kinase A in the maintenance of inflammatory pain. J. Neurosci., 19:2181-2186; and Khasar et al., (1999a) A novel nociceptor signaling pathway revealed in protein kinase C epsilon mutant mice. Neuron, 24:253-260). Whilst a mitogen-activated protein kinase/extracellularsignal related kinase kinase (MEK) second messenger signalling pathway has recently been shown to also contribute to epinephrineinduced hyperalgesia, these experiments were only performed in male rats (Aley, K. O., Martin, A., McMahon, T., Levine, J. D. & Messing, R. O., unpublished results). Taken together, these observations suggest that sex hormones and oestrogen in particular may directly infuence the function of primary afferent nociceptors. Epinephrine induces hyperalgesia that is mediated by PKCε and PKA. Since this action of epinephrine is mediated by action at b2-adrenergic receptors (Khasar et al., (1999b) Epinephrine produces a beta-adrenergic receptor-mediated mechanical hyperalgesia and in vitro sensitization of rat nociceptors. J. Neurophysiol., 81:1104-1112), whose density, agonist affinity and coupling to second messengers is controlled by sex hormones (Hatjis et al., (1989) Treatment of oophorectomized guinea pigs with intrauterine 17 beta-estradiol pellets may modulate myometrial beta-adrenergic receptor binding properties. Am. J. Obstet. Gynecol., 161:1628-1632; Shima, (1992) Effects of androgen on α- and β-adrenergic receptors in membranes from the rat seminal vesicle. Biochim. Biophys. Acta, 1175:123-127; Ungar et al., (1993) Estrogen uncouples beta-adrenergic receptor from the stimulatory guanine nucleotide-binding protein in female rat hypothalamus. Endocrinology, 133:2818-2826; Xu et al., (1993) Postreceptor events involved in the up-regulation of beta-adrenergic receptor mediated lipolysis by testosterone in rat white adipocytes. Endocrinology, 132:1651-1657; Alonso et al., (1995) Ovarian hormones regulate alpha 1- and beta-adrenoceptor interactions in female rat pinealocytes. Neuroreport, 6:345-348; and, Yie & Brown, (1995) Effects of sex hormones on the pineal response to isoproterenol and on pineal beta-adrenergic receptors. Neuroendocrinology, 62:93-100), we tested the hypothesis that sex hormones regulate the contribution of PKCε- and PKA-mediated signalling in epinephrine-induced inflammatory pain. Our findings indicate that PKCε, PKA and nitric oxide (NO) signalling pathways contribute to epinephrine-induced hyperalgesia in males but not in females, due to suppression by oestrogen, whilst MEK contributes in both sexes.

Materials and Methods

Animals

Behavioural experiments were performed on male and female Sprague-Dawley rats (Bantin and Kingman, Fremont, Calif., USA). Twenty-one day-old rats of either sex were gonadectomized and used in experiments when they were adults. In all other cases, same aged adult (250-350 g) rats were used. Male and female mice, wild-type or lacking expression of PKCε (Khasar et al., (1999a), supra), were employed in studies of mechanical nociception. Animals were housed in a controlled environment in the Animal Care Facility of the University of California, San Francisco, under a 12-h light/dark cycle. Food and water were available ad libitum. Care and use of animals conformed to NIH guidelines. Experimental protocols were approved by the UCSF Committee on Animal Research.

Gonadectomy

Three-week-old female rats were ovariectomized through bilateral upper flank incisions (Wayneforth & Flecknell, (1992). Experimental and Surgical Techniques in the Rat. Academic Press, London). The ovarian bundles were tied off with 4-0 silk sutures and the ovaries removed. The fascia was closed with 5-0 chromic gut and the skin closed with metal clips. Three-week-old male rats were castrated through a single scrotal incision (Wayneforth & Flecknell, (1992), supra). The testicular bundles were ligated with 4-0 chromic gut suture before removing the testes, and the skin closed with metal clips. These procedures were carried out under inhalational (2% isoflurane in oxygen; Matrix, Orchard Park, N.Y., USA) anaesthesia.

Administration of Oestrogen

Chronic administration of oestrogen was performed as described previously. Briefly, 17β-estradiol (Sigma, St. Louis, Mo., USA) was chronically administered by implants made from Silastic, tubing (1.67 mm inner diameter×3.18 mm outer diameter; Storz Instruments, St. Louis, Mo., USA) with a 5-mm length filled with oestrogen (Smith et al., (1977) Hormone administration: peripheral and intracranial implants. In Myers, R. D. (ed.), Methods in Psychobiology. Academic Press, New York, pp. 259-279). The ends of the implants were blocked with wooden sticks and sealed with Type A Silastic, medical adhesive (Dow Corning, Midland, Mich., USA). Implants were washed in absolute ethanol and equilibrated in four changes of warm phosphate-buffered saline over a 24-h period before placement in the rat. The implants were placed subcutaneously on the back, at the time of gonadectomy, and remained in place through the remainder of the experiment, to produce sustained levels of oestrogen over an extended period of time (Smith et al., (1977), supra).

Behavioural Experiments

The nociceptive flexion reflex was quantified using the Randall-Selitto paw pressure device (Analgesymeter, Stoetling, Chicago, Ill., USA), which applies a linearly increasing mechanical force to the dorsum of the rat's hind paw. The mechanical nociceptive threshold was defined as the force in grams at which the rat withdrew its paw. The protocols for this procedure have been previously described (Taiwo et al., (1989) The contribution of training to sensitivity in the nociceptive paw-withdrawal test. Brain Res., 487:148-151). Rats were familiarized in the testing procedure, at 5-min intervals for a period of 1 h per day for 3 days in the week preceding the experiment, to decrease variance in nociceptive thresholds (Taiwo et al., (1989), supra). Baseline paw-withdrawal threshold was defined as the mean of six readings before test agents were injected. Each paw was treated as an independent measure (Taiwo et al., (1989), supra) and each experiment was performed on a separate group of rats. Each group of rats was treated with only one agonist and/or antagonist injected peripherally by the intradermal route. It has been shown in previous studies that these agents, which are injected using the protocol described, exert a peripheral rather than central action (Khasar et al., (1995), supra; and, Khasar et al., (1999b), supra). The dose-response relationship for the effect of epinephrine was determined over a dose range of 1 ng-1 μg. All behavioural testing was done between 10.00 and 16.00 h. The blocking agents, with and without epinephrine (Khasar et al., (1999b), supra) or prostaglandin E₂ (PGE₂) (Aley et al., (1998), supra; Aley & Levine, (1999), supra; and, Aley et al., (2000) Chronic hypersensitivity for inflammatory nociceptor sensitization mediated by the epsilon isozyme of protein kinase C. J. Neurosci., 20:4680-4685), were injected as described previously (Khasar et al., (1995), supra; and, Khasar et al., (1999b), supra). The doses of the antagonists were shown in previous studies to produce a significant attenuation of epinephrine-induced hyperalgesia in male rats (Khasar et al., (1999b), supra). Because they are less membranepermeable, injections of the protein kinase inhibitors, PKCε inhibitor (PKCε-I) (Johnson et al., (1996) A protein kinase C translocation inhibitor as an isozyme-selective antagonist of cardiac function. J. Biol. Chem, 271:24962-24966) and Walsh inhibitor peptide (WIPTIDE; PKA inhibitor) (Dragland et al., (1985) Inhibition of cyclic AMP-dependent protein kinase-induced changes in the kinetic properties of hepatic pyruvate kinase by the specific cyclic AMP antagonist, (Rp)-diastereomer of adenosine cyclic 3′,5′-phosphorothioate. J. Cyclic Nucleotide Protein Phosphorylation Res., 10:371-382; and, Glass et al., (1989) Protein kinase inhibitor-(6-22)-amide peptide analogs with standard and nonstandard amino acid substitutions for phenylalanine 10. Inhibition of cAMP dependent protein kinase. J. Biol. Chem, 264:14579-14584), were always preceded by administration of 2.5 μL of distilled water in the same syringe to produce hypo-osmotic shock, thereby enhancing cell membrane permeability (Tsapis & Kepes, (1977) Transient breakdown of the permeability barrier of the membrane of Escherichia coli upon hypoosmotic shock. Biochim. Biophys. Acta, 469:1-12; West & Huang, (1980) Transient permeabilization induced osmotically in membrane vesicles from Torpedo electroplax: a mild procedure for trapping small molecules. Biochemistry, 19:4418-4423; Taiwo & Levine, (1989a) Contribution of guanine nucleotide regulatory proteins to prostaglandin hyperalgesia in the rat. Brain Res., 492:400-403; Khasar et al., (1995), supra; and, Widdicombe et al., (1996) Transient permeabilization of airway epithelium by mucosal water. J. Appl. Physiol., 81:491-499). The protein kinase inhibitor was separated from the distilled water by drawing up a small air bubble (<1 μL) into the syringe after drawing up the protein kinase inhibitor but before drawing up the distilled water.

Pharmacological Interventions

In this study, models of epinephrine- and prostaglandin E₂ (PGE₂)-induced hyperalgesia were used. Although epinephrine can activate both a- and β-adrenergic receptors (ARs), it has been demonstrated that epinephrine hyperalgesia in the rat is significantly attenuated by propranolol (a β-AR antagonist) but not phentolamine (an α-AR antagonist). The effect of propranolol (1 μg) on epinephrine (100 ng)-induced hyperalgesia was evaluated in intact male and female rats.

The effects of PKCε-I and WIPTIDE in both models have been previously described (Khasar et al., (1999b), supra) whilst the effect of the MEK inhibitors, PD 98059 and U 0126, was only recently studied on the epinephrine model (Aley K. O., Martin, A., McMahon, T., Levine, J. D. & Messing, R. O., unpublished results). Using the epinephrine model, the effects of PKCε-1 and WIPTIDE (both 1 μg) (Khasar et al., (1999b), supra) and MEK inhibitors, PD 98059 and U 0126 (each 1 μg) on epinephrine (100 ng)-induced hyperalgesia were tested in gonadintact and gonadectomized male and female rats to determine gender differences in the second messenger system(s) mediating this hyperalgesia. This dose of epinephrine was chosen based on dose-response studies demonstrating that 100 ng of epinephrine produced maximal hyperalgesia (Khasar et al., (1999b), supra). Since NO has been reported to contribute to PGE2-induced hyperalgesia by modulating PKA activity and since PKA has been shown to be involved in epinephrine-induced hyperalgesia, we sought to verify the contribution of NO in epinephrine-induced hyperalgesia in males and females by testing the effect of the NO synthetase (NOS) inhibitor NG-methyl-L-arginine (L-NMA; 1 μg) on PGE₂ (100 ng)- and epinephrine (100 ng)-induced hyperalgesia.

Von Frey Hair Stimulation in Mice

Mechanical nociceptive threshold in mice was determined by employing von Frey hair (VFH; Ainsworth, London, UK) stimulation of the plantar skin of each hind paw at intensities of 3.82 N/mm2 (36.3 mN) and 4.54 N/mm² (60.3 mN) using the up-and-down method (Chaplan et al., (1994) Quantitative assessment of tactile allodynia in the rat paw. J. Neurosci. Meth., 53:55-63; Kinnman & Levine, (1995) Involvement of the sympathetic postganglionic neuron in capsaicin-induced secondary hyperalgesia in the rat. Neuroscience, 65:283-291; and, Aley et al., (1996) Vincristine hyperalgesia in the rat: a model of painful vincristine neuropathy in humans. Neuroscience, 73:259-265). Normal and PKCε knockout mice were studied as described previously (Khasar et al., (1999a), supra), before and after epinephrine (100 ng) injection into the site on the plantar surface of the hindpaw to which VFH were applied.

Drugs

The following drugs used in this study were from Sigma (St. Louis, Mo., USA) unless otherwise noted: epinephrine (a β-AR agonist), PGE₂, propranolol, L-NMA; WIPTIDE (Penninsula Laboratories, Belmont, Calif., USA), PKCε inhibitor (Calbiochem, La Jolla, Calif., USA), PD 98059 (Calbiochem) and U 0126 (Calbiochem). Epinephrine (4 mg/mL) was dissolved in distilled water with an equivalent amount of ascorbic acid just before it was used and was kept on ice in subdued lighting conditions. A stock solution of PGE₂ (4 mg/mL) was made by dissolving it in 10% ethanol in normal saline; further dilution to 100 ng was made by addition of saline. Final concentration of ethanol was <10%. Propranolol, L-NMA, WIPTIDE and PKCε-I were dissolved in distilled water whilst PD 98059 and U 0126 (1 μg/μL) were dissolved in 10% dimethyl sulfoxide and diluted in distilled water before use. Stock solutions (1 μg/μL) of inhibitors were stored at −20° C. With the exception of oestrogen which was administered by subcutaneous implants, all injections were by the intradermal route in the hind paw as previously described (Taiwo & Levine, (1989a), supra; Taiwo & Levine, (1989b) Prostaglandin effects after elimination of indirect hyperalgesic mechanisms in the skin of the rat. Brain Res., 492:397-399; Taiwo et al., (1989), supra; Khasar et al., (1995), supra; and, Khasar et al., (1999b), supra). Inhibitors were injected at a concentration of 1 μg/2.5 μL.

Statistical Analysis

Data are presented as mean ±SEM values and compared using Student's t-test or ANOVA followed by Fisher's Protected Least Significant Difference (PLSD) post hoc test, as appropriate. A probability of P<0.05 was considered significant.

Results

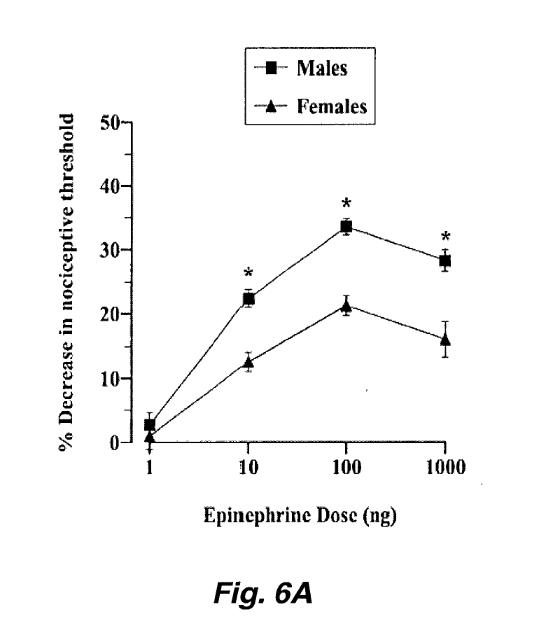

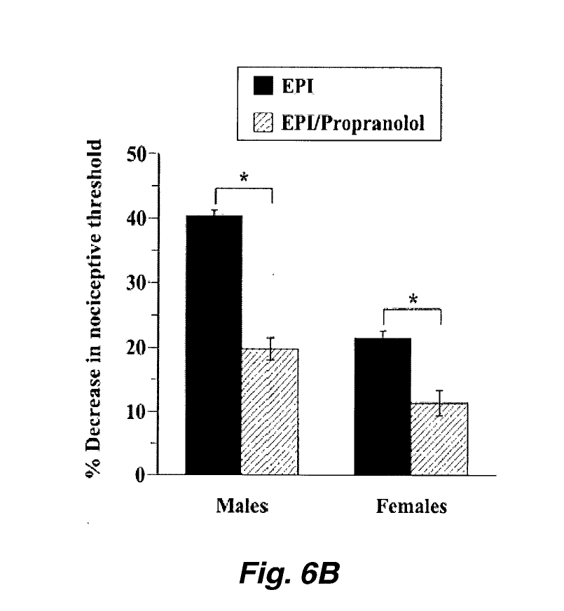

Hyperalgesic effect of epinephrine in normal rat Intradermal injection of epinephrine (1-1000 ng) into the dorsal surface of the hind paw of the rat produced a dose-dependent decrease in mechanical nociceptive threshold (i.e. hyperalgesia) in normal male and female rats (FIG. 6A). Preceding intradermal injection of epinephrine, the paw-withdrawal threshold was higher (P<0.0001) in males (107.9±1.2 g, n=36) compared to females (92.9±1.0 g, n=36). In addition, the absolute decrease in threshold after epinephrine was much greater (P<0.0001) in males (63.7±0.9 g, n=18) than in females (72.7±0.9 g, n=18). The β-adrenergic receptor antagonist propranolol (1 μg) inhibited epinephrine-induced hyperalgesia in both male and female rats. (both P<0.01; FIG. 6B).

Effect of Gonadectomy on Baseline and Epinephrine-Induced Hyperalgesia

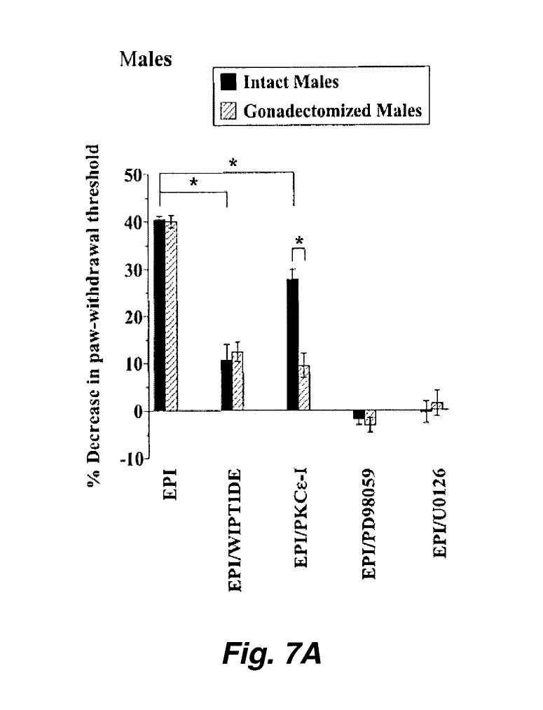

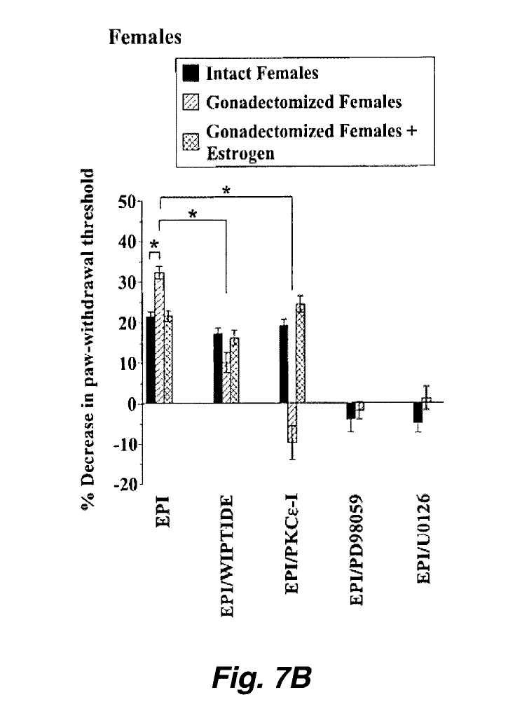

The baseline paw-withdrawal threshold was lowered by gonadectomy in both males (100.1±1.6 g, n=18 vs. 107.9±1.2 g, n=36, P<0.0001) and females (84.8±0.8 g, n=18 vs. 92.9±1.0 g, n=36, P<0.0001). Compared to gonad-intact females, gonadectomized females demonstrated a greater (P<0.0001) decrease in threshold after epinephrine (FIG. 7B). In contrast, there was no significant difference in the decrease in paw-withdrawal threshold produced by epinephrine in the gonadectomized males compared to gonad-intact males (FIG. 7A).

The Effect of Gonadectomy and Sex Hormone Replacement on PKCε and PKA Signalling in Epinephrine-Induced Hyperalgesia

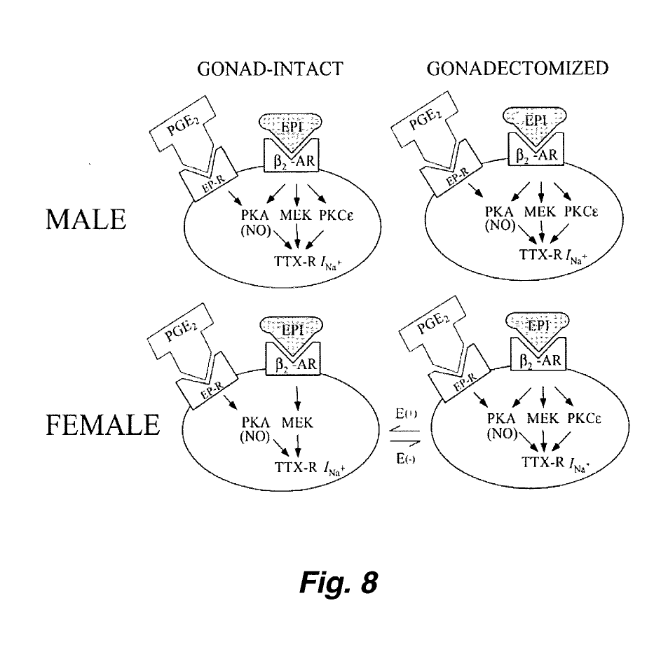

Whilst PKCE-I and WIPTIDE both antagonized epinephrine-induced hyperalgesia in intact male rats (P<0.0001; FIG. 7A), neither inhibited epinephrine hyperalgesia in intact female rats (FIG. 7B). Following gonadectomy, however, female rats developed a male phenotype, demonstrating a decrease in mechanical nociceptive threshold (92.9±1.0 g, n=36 in the intact female vs. 84.8±0.8 g, n=18 in the gonadectomized female), enhancement of epinephrineinduced hyperalgesia and inhibition of epinephrine-induced hyperalgesia by both PKCε-I and WIPTIDE (FIGS. 7B and 8). When female sex hormone was administered to gonadectomized females, the female phenotype was restored for all parameters (FIG. 8). Gonadectomized males had an unchanged hyperalgesic response to epinephrine compared to intact males (FIGS. 7A and 8) but a significantly greater inhibition of epinephrine hyperalgesia by PKCε-I (P<0.0001; FIG. 7A).

The Effect of L-NMA on Epinephrine-Induced Hyperalgesia

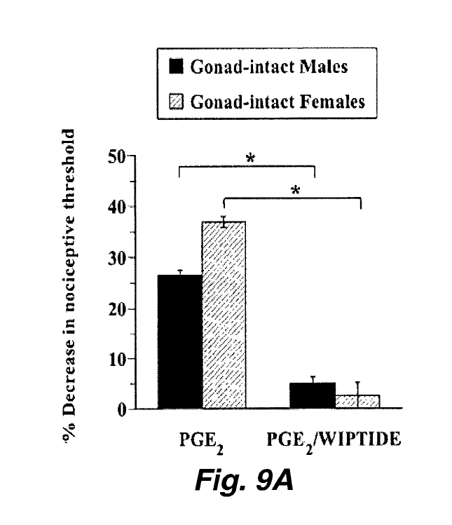

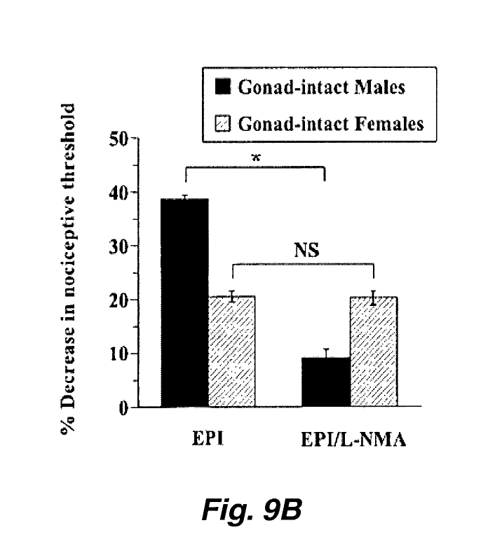

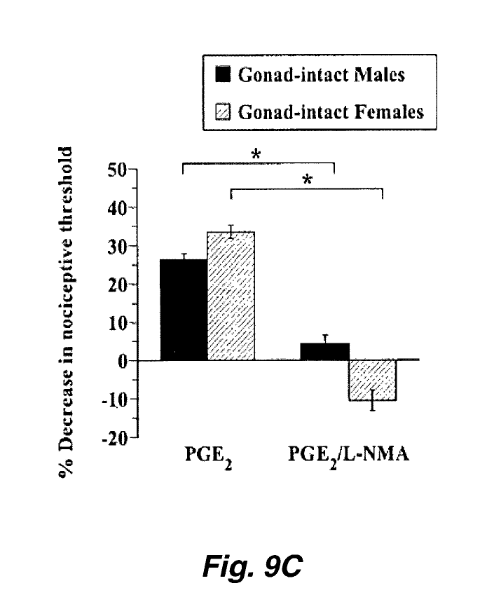

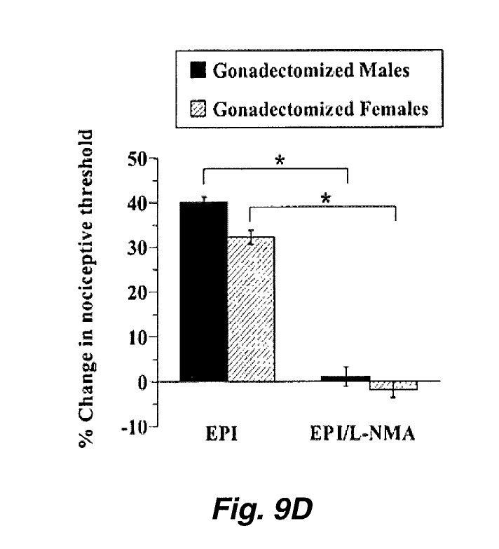

Whilst L-NMA significantly antagonized epinephrine-induced hyperalgesia in intact males (P<0.0001; FIG. 9B) and gonadectomized males and females (P<0.0001; FIG. 9D), it did not have a significant effect (P=0.85; FIG. 9B) in intact females.

The Effect of WIPTIDE and L-NMA on Prostaglandin E2-Induced Hyperalgesia

The magnitude of prostaglandin E₂-induced hyperalgesia in intact female rats was greater than that in males (P<0.0001; FIG. 9A and C); WIPTIDE (P<0.001; FIG. 9A) and L-NMA (P<0.01; FIG. 9C) significantly blocked prostaglandin E₂-induced hyperalgesia in both sexes.

MEK Signalling in Epinephrine-Induced Hyperalgesia.

Since in the male rat, signalling via MEK, independent of PKCε, PKA or NO (Aley K. O., Martin, A., McMahon, T., Levine, J. D. & Messing, R. O., unpublished results), contributes to epinephrine hyperalgesia, we tested whether MEK signalling contributed to epinephrine hyperalgesia in the female rat. We found that the MEK inhibitors PD 98059 or U 0126 also attenuated epinephrine hyperalgesia in the female rat (FIG. 7).

Von Frey Hair Stimulation in Mice

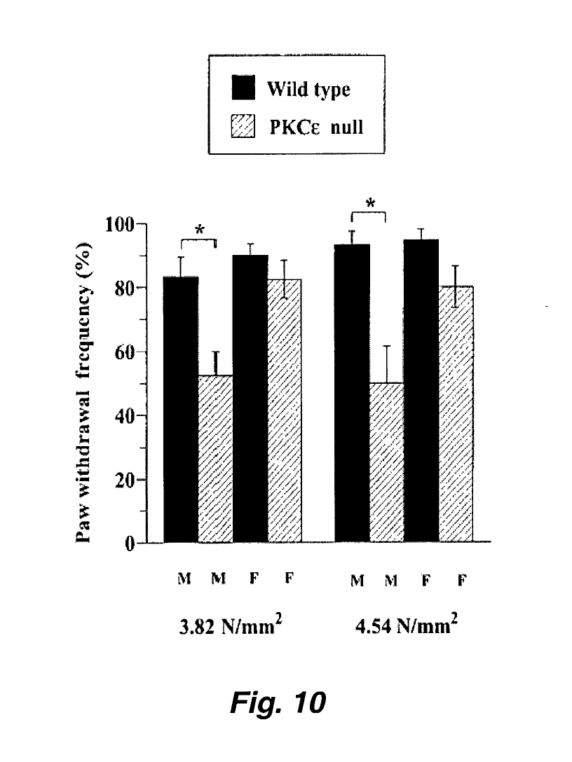

To confirm gender differences in the role of PKCε in epinephrine hyperalgesia, the effect of epinephrine on nociceptive threshold was examined in male and female null mutant mice. Evaluation of responses to VFH stimulation at different intensities was compared after epinephrine injection into the hind paw in wild-type and PKCε mutant male and female mice. Response to VFH stimulation intensities of 3.82 N/mm² (36.3 mN) and 4.54 N/mm² (60.3 mN) was similar in wild-type and mutant females administered epinephrine (FIG. 10). In contrast, the epinephrine-induced change in nociceptive response was significantly less in male PKCε-null mice (P<0.05, for both 3.82 and 4.54 N/mm²; FIG. 10).

Discussion

In this study we found that male rats demonstrate greater epinephrine-induced hyperalgesia than female rats. Furthermore, this response is sensitive to inhibitors of PKA, NOS and PKCε only in males. Gonadectomy unmasked sensitivity to PKA, NOS and PKCε inhibition in females and this was reversed by oestrogen replacement. These findings suggest that oestrogen decreases epinephrine-induced mechanical hyperalgesia in females by suppressing contributions of PKCε and PKA to pain signalling. Although gonadectomy did not alter the magnitude of the response to epinephrine in males, it did increase sensitivity to an inhibitor of PKCε, suggesting that testosterone may regulate PKCε-dependent nociception in male rats. Our results with PKCε-null mice support this notion, in that absence of PKCε was associated with decreased epinephrine-induced hyperalgesia only in male PKCε-null mice. The finding of a more prominent role for female sex hormone in epinephrine-induced hyperalgesia is consistent with gender differences observed in inflammation-induced pain (Angele et al., (1999) Sex steroids regulate pro- and anti-inflammatory cytokine release by macrophages after trauma-hemorrhage. Am. J. Physiol., 277:C35-C42; and, Da Silva et al., (1999) Gender differences in adrenal and gonadal responses to inflammatory aggression. Ann. N.Y. Acad. Sci., 876:148-151) and pain produced by formalin (Aloisi et al., (1994) Sex differences in the behavioural response to persistent pain in rats. Neurosci. Lett., 179:79-82; Aloisi et al., (1995) Sex-related effects on behaviour and beta-endorphin of different intensities of formalin pain in rats. Brain Res., 699:242-249; and, Nayebi & Ahmadiani, (1999) Involvement of the spinal serotonergic system in analgesia produced by castration. Pharmacol. Biochem. Behav., 64:467-471).

Whilst baseline thresholds (i.e. before injection of epinephrine) in gonadectomized male and female rats were significantly decreased, baseline values were not significantly different between gonadectomized females and oestrogen-replaced females. Furthermore, PKCε, PKA and NOS inhibitors did not affect baseline nociceptive threshold, in normal or gonadectomized rats (data not shown). Taken together, it would appear that the mechanism underlying the decrease in baseline nociceptive threshold produced by gonadectomy in male and female rats differs from that mediating enhanced hyperalgesia in females.

Since PGE₂-induced hyperalgesia is PKA-dependent in male rats (England et al., (1996) PGE₂ modulates the tetrodotoxin-resistant sodium current in neonatal rat dorsal root ganglion neurones via the cyclic AMP-protein kinase A cascade. J. Physiol. (Lond.), 495:429-440; Wang et al., (1996) Sensitization of C-fibers by prostaglandin E₂ in the rat is inhibited by guanosine 5′-O-(2- thiodiphosphate), 2′,5′-dideoxyadenosine and Walsh inhibitor peptide. Neuroscience, 71:259-263; Gold et al., (1998) Modulation of TTX-R INa by PKC and PKA and their role in PGE₂-induced sensitization of rat sensory neurons in vitro. J. Neurosci., 18:10345-10355; and, Aley & Levine, (1999), supra) and nitric oxide is thought to contribute to PGE₂-induced hyperalgesia and nociceptor sensitization (Aley & Levine, (1999), supra; and, Chen & Levine, (1999) NOS inhibitor antagonism of PGE2-induced mechanical sensitization of cutaneous C-fiber nociceptors in the rat. J. Neurophysiol., 81:963-966) by modulating PKA activity (Aley et al., (1998), supra), we evaluated the contribution of NO in epinephrine-induced hyperalgesia in male and female rats. Our experiments with the protein kinase A inhibitor, WIPTIDE and the NOS inhibitor L-NMA suggests that PKA signalling in primary afferent nociceptors is not regulated by sex hormones since PGE₂-induced hyperalgesia was inhibited to a similar degree in male and female rats by these inhibitors. In contrast, L-NMA did inhibit epinephrine-induced hyperalgesia in the intact male, but not female, rats, consistent with a role for PKA in hyperalgesic effects of epinephrine only in males. Since gender differences were apparent for the roles of PKA, NOS and PKCε in epinephrine-mediated hyperalgesia, but not in PGE₂- mediated hyperalgesia, it is likely that oestrogen regulates epinephrine-induced hyperalgesia at the level of the β-adrenergic receptor or the G-proteins to which it is coupled. In support of this hypothesis, ovariectomy has been reported to increase the density of β-adrenergic receptors (Hatjis et al., (1989), supra; and, Yie & Brown, (1995), supra), and to increase β adrenergic receptor-mediated responses in other cell types (Studer & Borle, (1982) Differences between male and female rats in the regulation of hepatic glycogenolysis. The relative role of calcium and cAMP in phosphorylase activation by catecholamines. J. Biol. Chem., 257:7987-7993; Yagami et al., (1994) The involvement of the stimulatory G protein in sexual dimorphism of beta-adrenergic receptor mediated functions in rat liver. Biochim. Biophys. Acta, 1222, 257-264; and, Yie & Brown, (1995), supra). This idea is also supported by reports that oestrogen can uncouple β-adrenergic receptors from their stimulatory G-proteins (Ungar et al., (1993), supra; Yagami et al., (1994), supra; and, Ansonoff & Etgen, (2000) Evidence that oestradiol attenuates betaadrenoceptor function in the hypothalamus of female rats by altering receptor phosphorylation and sequestration. J. Neuroendocrinol., 12:1060-1066). In contrast to male rats, epinephrine-induced hyperalgesia in female rats is not mediated by PKA, NO or PKCε signalling pathways. Since we have recently found that MEK is part of a novel signalling pathway, independent of PKA and PKCε, mediating epinephrine hyperalgesia (Aley K. O., Martin, A., McMahon, T., Levine, J. D. & Messing, R. O., unpublished results), we tested the hypothesis that epinephrine in the intact female rat is mediated by this pathway. A role for MEK was supported by the finding that two MEK inhibitors antagonized epinephrine hyperalgesia in the intact female. These data suggest that there may be sex differences in targets for control of hyperalgesic pain in males and females.

In summary, the findings of the present study demonstrate gender differences in PKCε, PKA and NO signalling to epinephrine-induced hyperalgesia. These results suggest that these differences are oestrogen-dependent and are regulated at the level of the β-adrenergic receptor or coupling of the receptor to heterotrimeric G-proteins.

Acknowledgements

This work was funded by NIH Grants NR 04880 and NS 21647. Abbreviations AR, adrenergic receptor; DRG, dorsal root ganglion; L-NMA, NG-methyl-Larginine; MEK, mitogen-activated protein kinase/extracellular-signal-related kinase kinase; NO, nitric oxide; NOS, nitric oxide synthetase; PGE₂, prostaglandin E₂; PKA, protein kinase A; PKC, protein kinase C; PKCε, epsilon isoform of protein kinase C; PKCε-I, protein kinase C epsilon inhibitor peptide; PLSD, Protected Least Significant Difference; VFH, von Frey hairs; WIPTIDE, Walsh inhibitor peptide.

### Example 3

Role of Ras-MEK-ERK 1/2 Cascade in Peripheral Neuropathy

The role of Ras-MEK-ERK 1/2 cascade in painful peripheral neuropathy has been evaluated in three rodent models of common clinical conditions in which patients experience painful peripheral neuropathy. The three models are as follows: 1) Taxol® (a major cancer chemotherapy agent)-induced painful peripheral neuropathy (Dina, O. A., et al., (2001) Role of protein kinase C-epsilon and protein kinase A in a model of paclitaxel-induced painful peripheral neuropathy in the rat. Neuroscience 108:507-515); 2) vincristine (another cancer chemotherapy agent)-induced painful peripheral neuropathy (Aley, K. O., et al., (1996) Vincristine hyperalgesia in the rat: a possible modelfor painful vincristine neuropathy in humans. Neuroscience 73:259-265; Tanner, K. D., et al., (1998) Nociceptor hyper-responsiveness during vincristine-induced painful peripheral neuropathy in the rat. J. Neuroscience 18:6480-6491; Tanner, K. D., et al., (1998) Microtubule disorientation and axonal swelling in unmyelinated sensory axons during vincristine-induced painful neuropathy in rat. J. Comp. Neurol., 395:481-492; and, Tanner, K. D., et al., (2002) Altered temporal patterning of afferent activity in a rat model of vincristine-induced peripheral neuropathy (Submitted -Neuroscience).); and, 3) alcohol-induced painful peripheral neuropathy (Dina, O. A., et al., (2000) Key role for the epsilon isoform of protein kinase C in painful alcoholic neuropathy in the rat. J. Neurosci. 20:8614-8619). In all three models, inhibition of MEK by a MEK inhibitor, PD98509, produced either a moderate (Taxol and vincristine) or profound (alcohol) reversal of the painful peripheral neuropathy. In the third model, alcohol-induced painful peripheral neuropathy, a second inhibitor of the Ras-MEK-ERK 1/2 cascade, which was MEK inhibitor U0126, was also employed.

Results

An Inhibitor of the Ras-MEK-ERK 1/2 Cascade Reduces Taxol-Induced Hyperalgesia

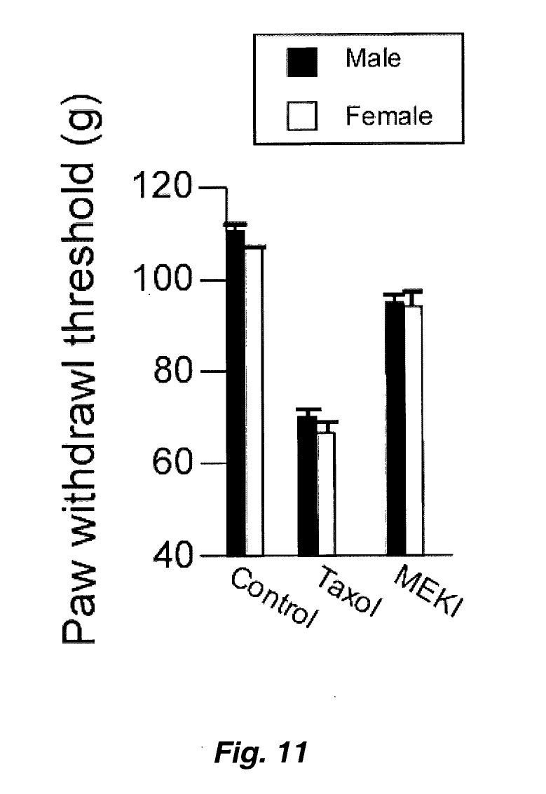

An inhibitor of MEK, PD98059 (“MEK-I”), produced a two-thirds reduction in Taxol-induced hyperalgesia in male and female rats. The pain threshold in normal male and female rats was approximately 110 grams, using the Randall-Selitto paw-withdrawal test (plotted as absolute change in nociceptive threshold). Following administration of Taxol, the threshold fell to approximately 70 grams. The administration of the Ras-MEK-ERK 1/2 cascade inhibitor, e.g., PD98059, at the site of nociceptive testing on the dorsal surface of the rats' paw significantly reversed the Taxol-induced hyperalgesia. See FIG. 11.

An Inhibitor of the Ras-MEK-ERK 1/2 Cascade Reduces Vincristine-Induced Hyperalgesia

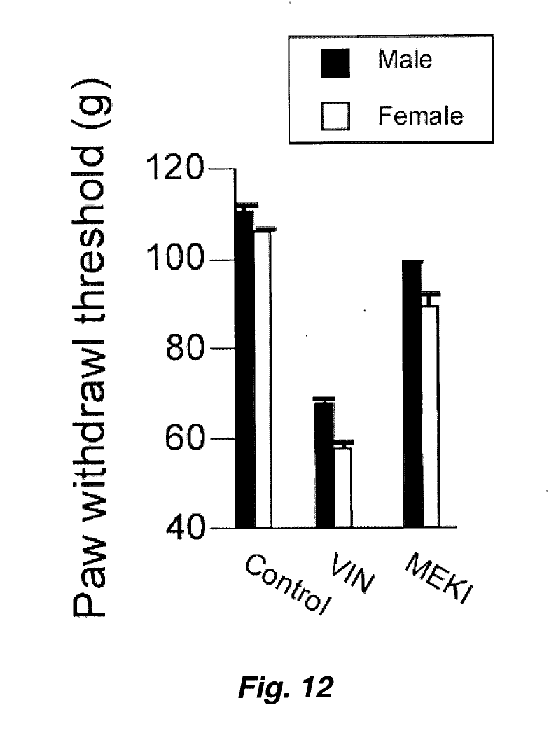

An inhibitor of Ras-MEK-ERK 1/2 cascade, MEK inhibitor PD98059, produced a more than two-thirds reduction in vincristine-induced hyperalgesia in male and female rats. The pain threshold in normal male and female rats was approximately 110 grams, using the Randall-Selitto paw-withdrawal test (plotted as absolute change in nociceptive threshold). Following administration of vincristine, the threshold fell to approximately 65 grams. The administration of the Ras-MEK-ERK 1/2 cascade inhibitor, PD980598, at the site of nociceptive testing on the dorsal surface of the rat's paw significantly reversed the vincristine-induced hyperalgesia. See FIG. 12.

Inhibitors of the Ras-MEK-ERK 1/2 Cascade Reduce Alcohol-Induced Hyperalgesia

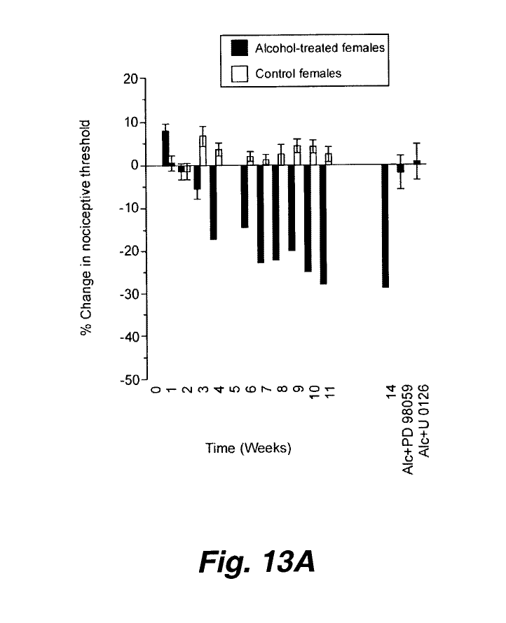

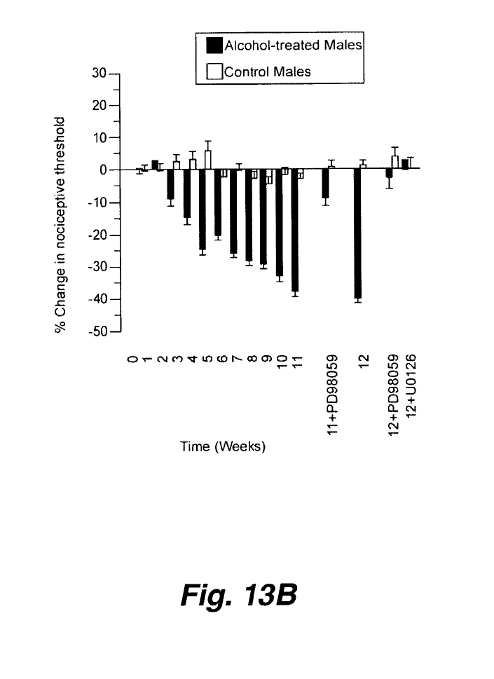

Inhibitors of the Ras-MEK-ERK 1/2 cascade, MEK inhibitors PD98059 and U0126, produced an almost complete reversal in alcohol-induced hyperalgesia in male and female rats. The pain threshold in normal rats was approximately 110 grams, using the Randall-Selitto paw-withdrawal threshold test (plotted as percentage change in nociceptive threshold). Following chronic consumption of a diet in which alcohol replaced calories, but not other nutrients, in the normal diet, the threshold fell to approximately 70 grams. The administration of Ras-MEK-ERK 1/2 cascade inhibitors, MEK inhibitors PD98059 or U0126, at the site of nociceptive testing on the dorsal surface of the rat's paw almost completely reversed alcohol-induced hyperalgesia. See FIG. 13A, which illustrates results from female rats and FIG. 13B, which illustrates results from male rats.

Discussion

In this study, we found that inhibition of the Ras-MEK-ERK 1/2 cascade produced marked reversal of three clinically relevant models of painful peripheral neuropathy. This therapy can be used in the treatment of a broad spectrum of painful peripheral neuropathies of diverse etiology.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of screening for an inhibitor of inflammatory or neuropathic pain, said method comprising:
assaying a test agent for the ability to inhibit pain that is mediated by a Ras-mitogen-activated protein kinase/extracelluar-signal related kinase kinase (MEK)-ERK 1/2 cascade.

2. The method of claim 1, wherein said assaying comprises:
providing a neurological tissue preparation;
contacting said neurological tissue with an agent that induces hyperalgesia;
contacting said neurological tissue with the test agent; and
assaying for expression or activity of a component of a Ras-mitogen-activated protein kinase/extracelluar-signal related kinase kinase (MEK)-ERK1/2 cascade, wherein a decrease in the expression or activity of said component as compared to the expression or activity of said component in a control assay indicates that said test agent inhibits inflammatory or neuropathic pain.

3. The method of claim 2, wherein said component is selected from the group consisting of ERK, MEK kinase, Ras protein, and a Gi/o protein.

4. The method of claim 3, wherein component is ERK.

5. The method of claim 3, wherein component is MEK kinase.

6. The method of claim 3, wherein component is a Ras protein.

7. The method of claim 1, wherein said assaying comprises:
providing a neurological tissue preparation;
contacting said neurological tissue with an agent that induces hyperalgesia;
contacting said neurological tissue with the test agent; and
assaying Gi/o protein expression or activity wherein a decrease in the Gi/o protein expression or activity as compared to a control indicates that said test agent inhibits inflammatory or neuropathic pain.

8. The method of claim 1, wherein said assaying comprises:
providing a neurological tissue preparation;
contacting said neurological tissue with an agent that induces hyperalgesia;
contacting said neurological tissue with the test agent; and
assaying nociceptive threshold activity wherein a decrease in a percentage decrease in the nociceptive threshold activity as compared to a control indicates that said test agent inhibits inflammatory or neuropathic pain.

9. The method of claim 2, wherein said control assay comprises an assay with the absence of said test agent or said test agent present at a lower concentration.

10. The method of claim 2, wherein said agent that induces hyperalgesia is epinephrine or NGF.

11. The method of claim 2, wherein said neurological tissue preparation is a dorsal root ganglion preparation.

12. The method of claim 2, wherein said neurological tissue preparation is a cell culture.

13. The method of claim 2, wherein said neurological tissue preparation is a brain slice.

14. The method of claim 2, further comprising assaying said test agent inhibitory or agonistic activity at PKA cascade or PKCε cascade where a lack of activity of said test agent at the PKA cascade or PKCε cascade indicates that said test agent is pathway-specific.

15. The method of claim 2, wherein said assaying comprises assaying for protein expression of a member of the Ras-MEK-ERK 1/2 cascade.

16. The method of claim 15, wherein said assaying comprises a method selected from the group consisting of: a capillary electrophoresis, a Western blot, mass spectroscopy, ELISA, immunochromatography, and immunohistochemistry.

17. The method of claim 2, wherein said assaying comprises assaying for a nucleic acid encoding a component of the the Ras-MEK-ERK 1/2 cascade.

18. The method of claim 17, wherein said nucleic acid is an mRNA.

19. The method of claim 17, wherein said nucleic acid is measured by hybridizing said nucleic acid to a nucleic acid probe that specifically hybridizes to a nucleic acid that encodes a compont of said Ras-MEK-ERK 1/2 cascade.

20. The method of claim 19, wherein said probe is a member of a plurality of probes that forms an array of probes.

21. The method of claim 19, wherein said hybridizing is according to a method selected from the group consisting of a Northern blot, a Southern blot using DNA derived from the EG-1 RNA, an array hybridization, an affinity chromatography, and an in situ hybridization.

22. The method of claim 17, wherein said assaying for a nucleic acid encoding a component of the the Ras-MEK-ERK 1/2 cascade comprises a a nucleic acid amplification reaction.

23. The method of claim 2, wherein said assaying comprises assaying for activity via a method selected from the group consisting of: a phosphorylation assay, an immunoassay, a binding assay and a nociceptive threshold assay.

24. The method of claim 1, wherein said test agent is contacted directly to the member of the Ras-MEK-ERK 1/2 cascade.

25. The method of claim 1, wherein said test agent is contacted to a cell comprising the Ras-MEK-ERK 1/2 cascade.

26. The method of claim 1, wherein said test agent is contacted to an animal comprising a cell containing the Ras-MEK-ERK 1/2 cascade.

27. The method of claim 1, wherein the assaying comprises:
selecting as the test agent, a compound that modulates the activity of the Ras-MEK-ERK 1/2 cascade; and,
administering the test agent to a subject to determine whether pain is modulated, wherein the test agent modulates pain in the subject by modulating the expression or activity of at least one member of the Ras-MEK-ERK 1/2 cascade.

28. A method of prescreening for an agent that inhibits pain mediated by a Ras-MEK-ERK 1/2 cascade, said method comprising:
contacting a member of the Ras-MEK-ERK 1/2 cascade, or a nucleic acid encoding a member of the Ras-MEK-ERK 1/2 cascade, with a test agent; and,
detecting specific binding of said test agent to said member of the Ras-MEK-ERK 1/2 cascade or to said nucleic acid encoding a member of the ras-MEK-ERK 1/2 cascade, wherein specific binding indicates that said agent is a candidate inhibitor of the Ras-MEK-ERK 1/2 cascade.

29. The method of claim 28, wherein said member is selected from the group consisting of ERK, MEK kinase, Ras protein, and a Gi/o protein.

30. The method of claim 28, further comprising recording test agents that specifically bind to said member of the Ras-MEK-ERK 1/2 cascade, or to said nucleic acid encoding a member of the ras-MEK-ERK 1/2 cascade, in a database of candidate agents that inhibits pain.

31. The method of claim 28, wherein said test agent is not an antibody.

32. The method of claim 28, wherein said test agent is not a protein.

33. The method of claim 28, wherein said test agent is not a nucleic acid.

34. The method of claim 28, wherein said test agent is a small organic molecule.

35. The method of claim 28, wherein said detecting comprises detecting specific binding of said test agent to said nucleic acid encoding a member of the ras-MEK-ERK 1/2 cascade

36. The method of claim 35, wherein said binding is detected using a method selected from the group consisting of a Northern blot, a Southern blot using DNA derived from a nucleic acid encoding a member of the Ras-MEK-ERK 1/2 , an array hybridization, an affinity chromatography, and an in situ hybridization.

37. The method of claim 28, wherein said detecting comprises detecting specific binding of said test agent to said member of the Ras-MEK-ERK 1/2 cascade.

38. The method of claim 37, wherein said detecting is via a method selected from the group consisting of capillary electrophoresis, a Western blot, mass spectroscopy, ELISA, immunochromatography, and immunohistochemistry.

39. The method of claim 28, wherein said test agent is contacted directly to the member of the Ras-MEK-ERK 1/2 cascade.

40. The method of claim 28, wherein said test agent is contacted to a cell containing the Ras-MEK-ERK 1/2 cascade.

41. A method of desensitizing nocioceptors, said method comprising inhibiting a Ras-MEK-ERK1/2 cascade.

42. The method of claim 41, wherein said inhibiting comprises inhibiting expression or activity of a component of said Ras-MEK-ERK1/2 cascade, wherein said component is selected from the group consisting of ERK, MEK kinase, Ras protein, and a Gi/o protein.

43. The method of claim 42, wherein said component is MEK kinase.

44. The method of claim 42, wherein said component is ERK.

45. The method of claim 42, wherein said component is Ras.

46. The method of claim 42, wherein said inhibiting comprises inhibiting Gi/o expression or activity.

47. The method of claim 41, wherein said inhibiting comprises inhibiting β₂ adrenergic receptor mediated expression or activation of ERK.

48. The method of claim 41, wherein said inhibiting comprises inhibiting NGF-mediated expression or activation of ERK.

49. The method of claim 41, wherein said inhibiting comprises inhibiting bradykinin-mediated expression or activation of ERK.

50. A method of reducing or lessening pain, the method comprising:
administering to a subject in need thereof, an effective amount of an inhibitor of a Ras-MEK-ERK 1/2 cascade wherein said effective amount is an amount sufficient to reduce said pain.

51. The method of claim 50, wherein said inhibitor inhibits a component of said Ras-MEK-ERK 1/2 cascade selected from the group consisting of ERK, MEK kinase, Ras protein, and a Gi/o protein.

52. The method of claim 50, wherein the subject is a human.

53. The method of claim 50, wherein the subject is a non-human mammal.

54. The method of claim 50, wherein the subject is a male.

55. The method of claim 50, wherein the subject is a female.

56. The method of claim 50, wherein the inhibitor is in a pharmaceutically acceptable excipient.

57. The method of claim 50, wherein the administration results in the subject having decreased hyperalgesia.

58. The method of claim 50, wherein the inhibitor is an inhibitor of a β₂ adrenergic receptor.

59. The method of claim 58, wherein the inhibitor of the β2 adrenergic receptor is an inverse agonist or an antagonist.

60. The method of claim 59, wherein the inverse agonist comprises ICI 118,551.

61. The method of claim 59, wherein the antagonist comprises propanolol.

62. The method of claim 50, wherein the inhibitor is an inhibitor of Gi/o protein activity.

63. The method of claim 62, wherein the inhibitor of the Gi/o protein activity is an isoprenylation inhibitor.

64. The method of claim 62, wherein the inhibitor of the Gi/o protein activity is a pertussis toxin.

65. The method of claim 62, wherein the inhibitor of the Gi/o protein activity is a perillic acid.

66. The method of claim 50, wherein the inhibitor is an inhibitor of Ras activity.

67. The method of claim 66, wherein the inhibitor of Ras activity is an inhibitor of farnesyltransferase.

68. The method of claim 66, wherein the inhibitor of Ras activity is a FTase I.

69. The method of claim 50, wherein the inhibitor is an inhibitor of MEK activity.

70. The method of claim 69, wherein the inhibitor of MEK activity is an U0126 or a PD98059.

71. The method of claim 50, wherein the inhibitor is an inhibitor of ERK 1/2 activity.

72. The method of claim 50, wherein the inhibitor is administered locally.

73. The method of claim 50, wherein the inhibitor is administered systemically.

74. The method of claim 50, wherein the inhibitor inhibits catalytic activity of a member of the Ras-MEK-ERK 1/2 cascade.

75. The method of claim 50, wherein the inhibitor inhibits intracellular translocation of a member of the Ras-MEK-ERK 1/2 cascade.

76. The method of claim 50, wherein the inhibitor acts directly on a member of the Ras-MEK-ERK 1/2 cascade.

77. The method of claim 50, wherein the inhibitor acts indirectly on a member of the Ras-MEK-ERK 1/2 cascade.

78. The method of claim 50, wherein the inhibitor is membranepermeable.

79. The method of claim 50, wherein the method further comprises administering at least one compound from the group consisting of: an inhibitor of cAMP, a nonsteroidal anti-inflammatory drug, a local anesthetic, an anticonvulsant, an antidepressant, and an opiod.

80. The method of claim 50, wherein said pain comprises inflammatory pain.

81. The method of claim 80, wherein the inflammatory pain is acute.

82. The method of claim 80, wherein the inflammatory pain is chronic.

83. The method of claim 80, wherein the inflammatory pain is due to a condition selected from the group consisting of: sunburn, osteoarthritis, colitis, carditis, dermatis, myositis, neuritis and collagen vascular disease.

84. The method of claim 50, wherein said pain comprises neuropathic pain.

85. The method of claim 84, wherein the neuropathic pain involves a peripheral nerve.

86. The method of claim 84, wherein the neuropathic pain involves a central nerve.

87. The method of claim 84, wherein the neuropathic pain is due to a neuropathy selected from the group consisting of: radiculopathy, mononeuropathy, mononeuropathy multiplex, polyneuropathy and plexopathy.

88. The method of claim 84, wherein the neuropathic pain is due to a condition selected from the group consisting of: causalgia, diabetes, collagen vascular disease, trigeminal neuralgia, spinal cord injury, brain stem injury, thalamic pain syndrome, cancer, chronic alcoholism, stroke, cancer, abscess, demyelinating disease, herpes infection, and AIDS.

89. The method of claim 84, wherein said neuropathic pain is due to one or more of the following selected from the group consisting of: trauma, surgery, amputation, toxin, and chemotherapy.

90. The method of claim 50, further comprising administering an inhibitor of a prostaglandin E₂ cascade to said mammal in a concentration sufficient to inhibit prostaglandin E₂ hyperalgesia.

91. The method of claim 90, wherein said inhibitor of the prostaglandin E₂ cascade is a nitric oxide synthetase (NOS) inhibitor.

92. The method of claim 91, wherein the NOS inhibitor is a N^{G}-methyl-L-arginine (L-MNA).

93. The method of claim 50, said method further comprising administering an inhibitor of a protein kinase A (PKA) cascade or protein kinase Cε (PKCε) cascade to said subject in a concentration sufficient to inhibit the PKA cascade or the PKCε cascade.

94. The method of claim 93, wherein the inhibitor of the PKA cascade is a Walsh inhibitor peptide (WIPTIDE) or a H89.

95. The method of claim 93, wherein the inhibitor is a PKCε inhibitor.

96. The method of claim 95, wherein the inhibitor of the PKCε cascade is a protein kinase C epsilon inhibitor peptide (PKCε-I), a calphostin C or an εV1-2.

97. The method of claim 50, wherein the inhibitor of Ras-MEK-ERK 1/2 cascade is administered transdermally.

98. The method of claim 50, wherein the inhibitor of Ras-MEK-ERK 1/2 cascade is administered as a cream, lotion or an emulsion.

99. The method of claim 50, wherein the inhibitor of Ras-MEK-ERK 1/2 cascade is administered topically.

100. A method of decreasing hyperalgesia or pain in a mammal, said method comprising administering estrogen or an estrogen analog or agonist to said mammal in a concentration sufficient to inhibit contributions of β adrenegic receptor mediated PKA or PKCε to pain signaling.

101. The method of claim 100, wherein the estrogen analog or agonist is selected from the group consisting of: an estradiol, an estrone, an ethinyl estradiol, a diethylstilbestrol, a mestranol, an estrone, a conjugated estrogen, a chlorotrianisene and analogs thereof.

102. The method of claim 100, wherein the mammal is a female mammal.

103. The method of claim 100, wherein the pain is inflammatory pain.

104. The method of claim 100, wherein the pain is neuropathic pain.

105. The method of claim 100, wherein the pain is of a type produced by formalin.

106. The method of claim 100, further comprising administering an inhibitor of a Ras-MEK-ERK 1/2 cascade to said mammal in a concentration sufficient to inhibit the Ras-MEK-ERK 1/2 cascade.

107. The method of claim 106, wherein the inhibitor of the Ras-MEK-ERK 1/2 cascade is a MEK inhibitor.

108. The method of claim 107, wherein the MEK inhibitor is a PD 98059 or an U0126.

109. The method of claim 107, wherein the Ras-MEK-EKR 1/2 inhibitor is a β2 adrenergic receptor inhibitor.

110. The method of claim 109, wherein the β2 adrenergic receptor is an inverse agonist or antagonist.

111. The method of claim 110, wherein the inverse agonist is an ICI 118,551.

112. The method of claim 110, wherein the antagonist is a propanolol.

113. The method of claim 100, further comprising administering an inhibitor of a prostaglandin E₂ cascade to said mammal in a concentration sufficient to inhibit prostaglandin E₂ hyperalgesia.

114. The method of claim 113, wherein said inhibitor is a NOS inhibitor.

115. The method of claim 114, wherein the NOS inhibitor is a N^{G}-methylL-arginine (L-MNA).

116. A method of screening for an inhibitor of inflammatory or neuropathic pain, said method comprising assaying a test agent for the abilito to modulate activity of a RAS-MEK-ERK 1/2 cascade and one or more pathways selected from the group consisting of a PKA cascade, and a PKC cascade.

117. The method of claim 116, wherein said method comprises screening said test agent according to the method of claim 1.

118. The method of claim 116, wherein said method further comprises screening said test agent for the ability to inhibit a PKA pathway.

119. The method of claim 116, wherein said method further comprises screening said test agent for the ability to inhibit a PKC pathway.

120. The method of claim 116, wherein said method comprises assaying aaid test agent for the ability to modulate activity of a tetrodotoxin-resistant sodium current wherein inhibition of the tetrodotoxin-resistant sodium current indicates that said test agent inhibits inflammatory or neuropathic pain mediated by a PKA cascade, a PKCε cascade and a RAS-MEK-ERK 1/2 cascade.

121. The method of claim 120, wherein said assaying comprises:
contacting a neurological tissue preparation with an agent that induces hyperalgesia;
contain said neurological tissue preparation with the test agent; and
assaying for modulation of the activity of the tetrodotoxin-resistant sodium current.

122. The method of claim 121, wherein the neurological tissue preparation comprises a neuronal culture.

123. The method of claim 122, wherein the neuronal culture is a primary neuronal culture.

124. The method of claim 121, wherein said neurological preparation is a dorsal root ganglion preparation.

125. The method of claim 121, wherein said agent that induces hyperalgesia is epinephrine or NGF.

126. The method of claim 121, wherein the agent that induces hyperalgesia is bradykinin or norepinephrine.

127. The method of claim 121, wherein the agent that induces hyperalgesia is prostaglandin E₂.

128. A composition for reducing pain, said composition comprising an inhibitor of a Ras-MEK-ERK 1/2 cascade.

129. The composition of claim 128, wherein said composition comprises an agent that inhibits activity or expression of a component of a a Ras-MIEK-ERK 1/2 cascade said component beign selected from the group consisting of of ERK, MEK kinase, Ras protein, and a Gi/o protein.

130. The composition of claim 128, wherein said agent is present in a pharmacologically acceptable excipient.

131. The composition of claim 130, wherein the composition is formulated for transdermal administration.

132. The composition of claim 130, where the composition is formulated for topical administration.

133. The composition of claim 130, where the composition is formulated as a cream, lotion or emulsion.

134. The composition of claim 128, wherein said composition comprises a unit doage formulation.

135. The composition of claim 128, wherein said composition further comprises an analgesic agent, the analgesic agent having a mechanism of action other than inhibition of the Ras-MEK-ERK 1/2 cascade.

136. The composition of claim 135, wherein the analgesic agent is selected from the group consisting of: an opioid, a local anesthetic, an anticonvulsant and an antidepressant.

137. The composition of claim 135, wherein the analgesic agent is a nonsteroidal anti-inflammatory drug (NSAID).

138. The composition of claim 137, wherein the NSAID is selected from the group consisting of: aspirin, ibuprofen, and indomethacin.

139. The composition of 128, further comprising an inhibitor of PKA cascade, an inhibitor of the PKCε cascade or both the inhibitor of the PKA cascade and the inhibitor of the PKCε cascade.

140. A kit for reducing pain, said kit comprising a container containing an inhibitor of a Ras-MEK-ERK 1/2 cascade.

141. The kit of claim 140, wherein said kit further comprises instructional materials teaching the use of an inhibitor of a Ras-MEK-ERK 1/2 cascade to reduce pain.
